SEQUENCE LISTING (6 sequences)

SEQ ID NO: 1 - DNA, 6690 bp, Mus musculus
cagcaagggcttgcaggtgcactagttctttctgtttttgtttgtgcggaaaggggcggccacagagcccgtgacggacaggctccagagtcgaccaagtgcttgcttgggaattctgtccagacagtgtttttgtaagaggacaaagctacttagtcccagccatgaggaaacccagtgagtatttggtcgcatagggcatggagctggagaactatgaacaaccggtggttctaagagaggacaacctccgccggcgccggaggatgaagccacgcagcgcagcaggcagcctgtcttccatggagctcatccccattgagttcgtactgcccaccagccagcgcatcagcaagactccagaaacagcgctgctgcatgtggctggccatggcaatgtggaacagatgaaagctcaggtgtggctgcgcgcactggagaccagtgtggctgcggagttctaccaccgattgggcccggaccaattcctcctgctctaccagaagaaaggacaatggtatgagatctatgacaggtaccaagtggtgcagaccctagactgcctgcattactggaagttgatgcacaagagccctggccagatccacgtggtacagcgacacgtaccttctgaggagaccttggctttccagaagcagctcacctccctgattggctatgacgtcactgacatcagcaatgtgcacgatgatgagctagagttcactcgccgccgtctggttacgccccgcatggctgaagtggctggccgggatgccaaactctatgctatgcacccttgggtaacgtccaaacctctcccagactacctgtcaaaaaagattgccaacaactgcatcttcatcgtcatccaccgcggtaccaccagccaaaccatcaaggtctccgcagatgatactcctggtaccatcctccagagcttcttcaccaagatggccaagaagaagtccctaatgaatatctcagaaagtcaaagtgagcaggattttgtattgcgggtttgtggccgcgatgagtacctggtgggtgaaacacccctcaaaaatttccagtgggtgaggcagtgcctcaagaacggagatgaaatacacctggtgctcgacacgcctccagacccagcccttgatgaggtgaggaaggaagaatggccgctggtggatgactgcactggagtcaccggctaccacgagcagctgaccatccatggcaaggaccacgagagtgtgttcacagtgtctttgtgggactgcgaccgaaagttcagggtcaagatcagaggcattgatatccctgtcctgcctcggaacaccgacctcactgtgtttgtggaagcgaacatccagcacgggcaacaagtcctctgccaaaggagaaccagccctaagcccttcgcagaagaggtactctggaatgtgtggctggagtttggcatcaaaatcaaagacttgcccaaaggggctctattgaacctacagatctactgctgcaaaaccccatcactgtccagcaaggcttctgcagagactccaggctccgagtccaagggcaaagcccagcttctctattacgtgaacttgctgttaatagaccaccgtttcctcctccgccacggggactatgtgctccacatgtggcagatatctggcaaggcagaggagcagggcagcttcaatgctgacaagctcacatccgcaaccaatcctgacaaggagaactcaatgtccatttccatcctgctggacaattactgtcaccccatagctttgcctaagcaccggcccacccctgacccagagggagacagggttcgggctgaaatgcccaatcagcttcgaaagcaattggaggcgatcatagccacagatccacttaaccccctcacagcagaggacaaagaattgctctggcattttcgatatgaaagcctgaagcatccgaaggcttaccctaagctattcagctcagtgaaatgggggcagcaagaaattgttgccaaaacgtaccagctgttagccagaagggagatctgggatcaaagtgctttggacgttggcttaaccatgcagctcctggactgcaacttttcagacgagaatgtccgggccattgcagttcagaaactggagagcttagaggacgatgacgttttacattaccttctccagctggtacaggctgtgaaatttgaaccgtaccacgacagtgcgctggccagattcctgctgaagcgtggcttgaggaacaaaagaatcggtcacttcttgttctggttcctgcgaagtgagatcgcacagtccagacactatcagcagaggttcgctgtgatcctggaggcgtacctgcgaggctgtggcacagccatgttgcaggacttcacacagcaggtccatgtgattgagatgttacagaaagtcaccattgatattaaatcgctctcggcagagaagtatgacgtcagttcccaagttatttcacagcttaagcaaaagcttgaaagccttcagaactccaatctccccgagagctttagagttccctatgatcctggactaaaagccggtaccctggtgatcgagaaatgcaaagtgatggcctccaagaagaagcccctgtggcttgagtttaagtgtgctgatcccacagtcctatccaacgaaaccattggaatcatctttaaacatggtgatgatctgcgccaagacatgttgatcttgcagattctacgcatcatggagtccatttgggagactgaatctctggacctgtgccttctgccttacggttgcatctcaactggtgacaaaataggaatgatcgagattgtaaaggatgccacaacgatcgctcaaattcagcaaagcacagtgggtaacacgggggcattcaaagatgaagtcctgaatcactggctcaaggaaaaatgtcctattgaagaaaagtttcaggccgcagtggaaaggtttgtttactcctgtgcaggctactgtgtggccacatttgttcttgggatcggtgacaggcacaacgacaacattatgatctcagagacaggaaacctatttcatatagacttcggacacattcttgggaattacaagagtttcctgggcatcaataaagagagagtgcccttcgtcctaaccccagacttcttgtttgtgatgggatcttctggaaaaaagacaagtccacacttccagaaattccaggatgtctgtgttagagcttacctagctcttcgccatcacacaaacctgttgatcatcttgttctccatgatgctgatgacaggaatgccccagctgacaagcaaagaggacattgaatatatccgggatgccctcaccgtgggaaaaagcgaggaggacgctaagaaatatttccttgatcagatcgaagtctgcagagacaaaggatggactgtgcagtttaactggttcctacatcttgttcttggcatcaaacaaggagaaaagcactccgcttgatatgtggtgctagggcccaaagcaatgtagtgttctggagcttttagatcagcatgtcaatcatcagcgttaagatttaaaacgcaataagactctctgaacactgtacttcagaaatccagctctttccccagctgaactcttcaccagagggggaaaaaaatgttggcattactgatggtttggttaatattcagtgcgaggcttctctgttccttttccctcctctcagtctccatgatgttgaagaatattctcagtttaaatagcttagagcagccttgcttagtatgcctgacgatctcgtattgagtatctctgaaagccctttagttagaataactggcaatacctgttttctcgtgtgtccaccatgtattttggctctccttgtgttctctaagctcttggcagaaaaaggtgcaagcattgctaagctctgctgcattctttaaagggtgtttctaagcatcccctcagtgcccacagagcaaggaatgcaagttagctcatagaataccagagtactgtcagcagtttgtttcccacagtataccactgagatgcccaggcactgaacacaagctctgagcctgagccttgcccctccccagctgctgtggctctagggttatggacgtgaaaagtggaggtgacaccttttaccaagagcgaataacctattacagccagtgtcatcttctcctgcacaaagcaggaagtcgctagctggacccctggatgcccacaaatgtcatctttccctcttgttctgttcccccttacccatgctgcaacacttcctgtacttcttcctggacttgtggactcttcaaagacatctcaaaatgacttgaaaaagaaagtctactctaggctcaggagagttataggtacgtatttgtggagccgttccatggatttttaaaaagaatattattttaaaaaaaaaaagagttccagaaaatactagtttctaaatgctttaattttacattttaaatctgagaagtttttctttattttttaatagaagatacctgctatcaaatgtattctaagcttataacaatacttattaggtattcattttttcagtttaaaggactttaaattttgaggcatcccagagttcataaagacaattgaaccaatttcctttgaagtactttttcatttcttaaatattatgctgtttgtcatcagtgttgatccacctttgagaggaatcagtaaattatttaatcaaatatctttagctatcattaattccatagaaagaaagatgtcaaagatgtccatatttagatataatcagatataattctttctgatttttaaattgtcaaaatagacacttccattagaaggaacccaacattccctttgtgtctccagaagccaagtagcacctaattctgctacaaataagaaccatcagggacactttcaacatagccttgcattttaaagtcaccccactttcagattataatttcattagtgtgaatgagcctgggtatggttttctaagcactgtggctgaatctaacacacatgcaggggaaggaggcttatttggtgcgtgatggtataggtaaccctgcctccttgcggactggaccttccctggctcataacgagctacaaaaacatctcctcatccctgggaagtaactgttctaaaactacgctgtctcctgtacaggccatgccctgtgctgaaactgtgcttctctctatgaatgtaattttatggcatttctggagtgtgattcctgctaatctaagaacaggacaaatgggacgcaagcttaggcttctaaagaaccatacacagactctgcctttcatttgaattgctgaaacgatgatcttgatgataaaacaattacggagtatatctatacttagcttccgacaccacatgcatacatattagttctttaaatgagccagtcttttcataaggctgaactacatggcgccgtagactgggtttcattcttccatttcccaaagaatgcaatttcaactccatagacttgcatataagtaaaagccaccttcatatgctaccttgccattcctgtgagtgatattgtccagtgatttctacacctttgaaaataatctagacctagggtctccaggtaaataccaatatctctaccttcagtgctgagtgaagaaaacagatcacaggtcagatggatcgggttaaagcaacttatgtgtgtatttaagacaacaaaagacacaaagcttccgttgtagacttggtccaatatgactctactggttttggtggtgctctgaaaaatgcagaaagacattatgaataaaatatatcaaccagaaaaagaaaaaagctctagacttgcaaataaattaagtatagtaagtccatctttttcaggagacaagttgttttggttttattagttctttgagaattgtgtacaaagagttttgaccatatgcacccacttccccaacatctcccagattcaccccctttccctgcccacccaattgtgtcctctctttttttttctttttgtccatcaatggcaacactcttcaagaaaacttcctctctccgcagctatcaattgccaacagttcttggctaggggtggaacttcatatccacctcccctctccatgctgggatttggtgcacgcgccctgagctttcctggggcttgtcctagctgctgtgagttcatatgtgcagctgccctgccatgtccagaggatcctgcttcctcgtattcgttcaccacctctgtctctcaccgtctctccaggtcctcttccaatgatcactgagccttgggaggagatggggaggtaatagatgctccatttagggctgagtattctgcaggctcttactctctgcaccaggactagctatgaatctcagtggtgatcatcatctatgcaaacagacactgctctgatgcaggctgagagatatactaatctatagataaaacagcaagtcatgaagggtcattttgggaattctatagcatgatttggaagctgttagtcctatattaatgacaattgaatataatcttaaataaaaaaattagaaaaaacatttctgaaatcatcagcaattatgtttaacaacgtgaagaaaataaaatgagctaggatttcttaa

SEQ ID NO: 2 - protein, 1102 aa, Mus musculus
MELENYEQPVVLREDNLRRRRRMKPRSAAGSLSSMELIPIEFVLPTSQRISKTPETALLHVAGHGNVEQMKAQVWLRALETSVAAEFYHRLGPDQFLLLYQKKGQWYEIYDRYQVVQTLDCLHYWKLMHKSPGQIHVVQRHVPSEETLAFQKQLTSLIGYDVTDISNVHDDELEFTRRRLVTPRMAEVAGRDAKLYAMHPWVTSKPLPDYLSKKIANNCIFIVIHRGTTSQTIKVSADDTPGTILQSFFTKMAKKKSLMNISESQSEQDFVLRVCGRDEYLVGETPLKNFQWVRQCLKNGDEIHLVLDTPPDPALDEVRKEEWPLVDDCTGVTGYHEQLTIHGKDHESVFTVSLWDCDRKFRVKIRGIDIPVLPRNTDLTVFVEANIQHGQQVLCQRRTSPKPFAEEVLWNVWLEFGIKIKDLPKGALLNLQIYCCKTPSLSSKASAETPGSESKGKAQLLYYVNLLLIDHRFLLRHGDYVLHMWQISGKAEEQGSFNADKLTSATNPDKENSMSISILLDNYCHPIALPKHRPTPDPEGDRVRAEMPNQLRKQLEAIIATDPLNPLTAEDKELLWHFRYESLKHPKAYPKLFSSVKWGQQEIVAKTYQLLARREIWDQSALDVGLTMQLLDCNFSDENVRAIAVQKLESLEDDDVLHYLLQLVQAVKFEPYHDSALARFLLKRGLRNKRIGHFLFWFLRSEIAQSRHYQQRFAVILEAYLRGCGTAMLQDFTQQVHVIEMLQKVTIDIKSLSAEKYDVSSQVISQLKQKLESLQNSNLPESFRVPYDPGLKAGTLVIEKCKVMASKKKPLWLEFKCADPTVLSNETIGIIFKHGDDLRQDMLILQILRIMESIWETESLDLCLLPYGCISTGDKIGMIEIVKDATTIAQIQQSTVGNTGAFKDEVLNHWLKEKCPIEEKFQAAVERFVYSCAGYCVATFVLGIGDRHNDNIMISETGNLFHIDFGHILGNYKSFLGINKERVPFVLTPDFLFVMGSSGKKTSPHFQKFQDVCVRAYLALRHHTNLLIILFSMMLMTGMPQLTSKEDIEYIRDALTVGKSEEDAKKYFLDQIEVCRDKGWTVQFNWFLHLVLGIKQGEKHSA

SEQ ID NO: 3 - DNA, 5379 bp, Homo sapiens
gcacttccttctcggctagattatctgaaactgttgtcggttcttgagatgatactaccaccgaatgtctgtgtttcattgtctagtccaacctgtattgtggatatctacaacgttccggcaatagttttgcaggtgcatcacatttttgtttttgttttgggaggaaaagggagggcacggcagccaggcttcatattcctacaagtgcatgcttcaagattactgtacttacagtgtttccaacatcttctcataaaaggggaaagcttcatagcctcaaccatgaaggaaaccagtcgcatagggcatggagctggagaactataaacagcccgtggtgctgagagaggacaactgccgaaggcgccggaggatgaagccgcgcagtgctgcggccagcctgtcctccatggagctcatccccatcgagttcgtgctgcccaccagccagcgcaaatgcaagagccccgaaacggcgctgctgcacgtggccggccacggcaacgtggagcagatgaaggcccaggtgtggctgcgagcgctggagaccagcgtggcggcggacttctaccaccggctgggaccgcatcacttcctcctgctctatcagaagaaggggcagtggtacgagatctacgacaagtaccaggtggtgcagactctggactgcctgcgctactggaaggccacgcaccggagcccgggccagatccacctggtgcagcggcacccgccctccgaggagtcccaagccttccagcggcagctcacggcgctgattggctatgacgtcactgacgtcagcaacgtgcacgacgatgagctggagttcacgcgccgtggcttggtgaccccgcgcatggcggaggtggccagccgcgaccccaagctctacgccatgcacccgtgggtgacgtccaagcccctcccggagtacctgtggaagaagattgccaacaactgcatcttcatcgtcattcaccgcagcaccaccagccagaccattaaggtctcacccgacgacacccccggcgccatcctgcagagcttcttcaccaagatggccaagaagaaatctctgatggatattcccgaaagccaaagcgaacaggattttgtgctgcgcgtctgtggccgggatgagtacctggtgggcgaaacgcccatcaaaaacttccagtgggtgaggcactgcctcaagaacggagaagagattcacgtggtactggacacgcctccagacccggccctagacgaggtgaggaaggaagagtggccgctggtggatgactgcacgggagtcaccggctaccatgagcagcttaccatccacggcaaggaccacgagagtgtgttcaccgtgtccctgtgggactgcgaccgcaagttcagggtcaagatcagaggcattgatatccccgtcctgcctcggaacaccgacctcacagtttttgtagaggcaaacatccagcatgggcaacaagtcctttgccaaaggagaaccagccccaaacccttcacagaggaggtgctgtggaatgtgtggcttgagttcagtatcaaaatcaaagacttgcccaaaggggctctactgaacctccagatctactgcggtaaagctccagcactgtccagcaaggcctctgcagagtcccccagttctgagtccaagggcaaagttcagcttctctattatgtgaacctgctgctgatagaccaccgtttcctcctgcgccgtggagaatacgtcctccacatgtggcagatatctgggaagggagaagaccaaggaagcttcaatgctgacaaactcacgtctgcaactaacccagacaaggagaactcaatgtccatctccattcttctggacaattactgccacccgatagccctgcctaagcatcagcccacccctgacccggaaggggaccgggttcgagcagaaatgcccaaccagcttcgcaagcaattggaggcgatcatagccactgatccacttaaccctctcacagcagaggacaaagaattgctctggcattttagatacgaaagccttaagcacccaaaagcatatcctaagctatttagttcagtgaaatggggacagcaagaaattgtggccaaaacataccaattgttggccagaagggaagtctgggatcaaagtgctttggatgttgggttaacaatgcagctcctggactgcaacttctcagatgaaaatgtaagagccattgcagttcagaaactggagagcttggaggacgatgatgttctgcattaccttctacaattggtccaggctgtgaaatttgaaccataccatgatagcgcccttgccagatttctgctgaagcgtggtttaagaaacaaaagaattggtcactttttgttttggttcttgagaagtgagatagcccagtccagacactatcagcagaggttcgctgtgattctggaagcctatctgaggggctgtggcacagccatgctgcacgactttacccaacaagtccaagtaatcgagatgttacaaaaagtcacccttgatattaaatcgctctctgctgaaaagtatgacgtcagttcccaagttatttcacaacttaaacaaaagcttgaaaacctgcagaattctcaactccccgaaagctttagagttccatatgatcctggactgaaagcaggagcgctggcaattgaaaaatgtaaagtaatggcctccaagaaaaaaccactatggcttgagtttaaatgtgccgatcctacagccctatcaaatgaaacaattggaattatctttaaacatggtgatgatctgcgccaagacatgcttattttacagattctacgaatcatggagtctatttgggagactgaatctttggatctatgcctcctgccatatggttgcatttcaactggtgacaaaataggaatgatcgagattgtgaaagacgccacgacaattgccaaaattcagcaaagcacagtgggcaacacgggagcatttaaagatgaagtcctgaatcactggctcaaagaaaaatcccctactgaagaaaagtttcaggcagcagtggagagatttgtttattcctgtgcaggctactgtgtggcaacctttgttcttggaataggcgacagacacaatgacaatattatgatcaccgagacaggaaacctatttcatattgacttcgggcacattcttgggaattacaaaagtttcctgggcattaataaagagagagtgccatttgtgctaacccctgacttcctctttgtgatgggaacttctggaaagaagacaagcccacacttccagaaatttcaggacatctgtgttaaggcttatctagcccttcgtcatcacacaaacctactgatcatcctgttctccatgatgctgatgacaggaatgccccagttaacaagcaaagaagacattgaatatatccgggatgccctcacagtggggaaaaatgaggaggatgctaaaaagtattttcttgatcagatcgaagtttgcagagacaaaggatggactgtgcagtttaattggtttctacatcttgttcttggcatcaaacaaggagagaaacattcagcctaatactttaggctagaatcaaaaacaagttagtgttctatggtttaaattagcatagcaatcatcgaacttggatttcaaatgcaatagacattgtgaaagctggcatttcagaagtatagctcttttcctacctgaactcttccctggagaaaagatgttggcattgctgattgtttggttaagcaatgtccagtgctaggattatttgcaggtttggttttttctcatttgtctgtggcattggagaatattctcggtttaaacagactaatgacttccttattgtccctgatattttgactatcttactattgagtgcttctggaaattctttggaataattgatgacatctattttcatctgggtttagtctcaattttggttatctttgtgttcctcaagctctttaaagaaaaagatgtaatcgttgtaacctttgtctcattccttaaatgatgcttccaaacatctccttagtgtctgcaggtgttagtggtgtgctaaaagcaaggaaagcgagttagtcttttcagtgtcttttgcaattcaattcttttgtcatgtataactgagacacacaaacacagcaggagaaatctaaaccgttgtgccttgaccttcctctgctggtcttgttccagggttatgaatatgaaaaaatagagatgagactttttgtgtcaactctgtccacaagagtgagttatctagtatgattagtatagctttctccagcatggcagcaggaagtaactacagggcctcttttatgcctgacatttcttcccttcctttttccctgcctccctttttcatcaattgcaatgctcccacaactctttacagacttgtgaaatcttcaagaacacctttactctataactcaaaaattagttgaaaaataattacttctcaaggattattagaatcttaggtacttatttgtaaagatgtttagtgacttttttttcaagtatcttattaaaggaggcattctagaaaatatgaattagtttccaaatgccttaattttaaactttggcctgaacagttttttctttttcttaatggaagaagatatttaatatcttaaaaatattccaagttaggaagaacactacttgccttatccatttcccatttaaaggacttttaaactttgacacatccttcagatttcctgaaaataattgaaatatcttactttaaaaatattttcatctctgaaatatctcgttatttattggaggtattgtttaaccttagagagaccattaaattatttataaaatattttgtaattacctgtagctaatacattacatagaaaaaaactatgttaacagtgtctctgtttaagtataatcagatataaatatatacttaattttttaattttaaaaatagatacctgtttgactttgaggtagtccagaccttttctttttttttttttttttaatgtgtgcaaaagcccaaaggttcctaagcctggctgcaaagaagaatcaacagggacactttttaaaaacactcttatcagcctgggcaacacagtgagactccatctcttaaaaaaaaaattagctgggtatagtggtatgtgcctgtagtcccaggtactcaggaggctgaggcaggaggattgcctgagcccaggaggtggaaactgcagagagtcatgatcatgtccttacactccagcctggataacagagcgagaccctgtctcaaaaaaataaaataaaaaataaaaaca

SEQ ID NO: 4 - protein, 1102 aa, Homo sapiens
MELENYKQPVVLREDNCRRRRRMKPRSAAASLSSMELIPIEFVLPTSQRKCKSPETALLHVAGHGNVEQMKAQVWLRALETSVAADFYHRLGPHHFLLLYQKKGQWYEIYDKYQVVQTLDCLRYWKATHRSPGQIHLVQRHPPSEESQAFQRQLTALIGYDVTDVSNVHDDELEFTRRGLVTPRMAEVASRDPKLYAMHPWVTSKPLPEYLWKKIANNCIFIVIHRSTTSQTIKVSPDDTPGAILQSFFTKMAKKKSLMDIPESQSEQDFVLRVCGRDEYLVGETPIKNFQWVRHCLKNGEEIHVVLDTPPDPALDEVRKEEWPLVDDCTGVTGYHEQLTIHGKDHESVFTVSLWDCDRKFRVKIRGIDIPVLPRNTDLTVFVEANIQHGQQVLCQRRTSPKPFTEEVLWNVWLEFSIKIKDLPKGALLNLQIYCGKAPALSSKASAESPSSESKGKVQLLYYVNLLLIDHRFLLRRGEYVLHMWQISGKGEDQGSFNADKLTSATNPDKENSMSISILLDNYCHPIALPKHQPTPDPEGDRVRAEMPNQLRKQLEAIIATDPLNPLTAEDKELLWHFRYESLKHPKAYPKLFSSVKWGQQEIVAKTYQLLARREVWDQSALDVGLTMQLLDCNFSDENVRAIAVQKLESLEDDDVLHYLLQLVQAVKFEPYHDSALARFLLKRGLRNKRIGHFLFWFLRSEIAQSRHYQQRFAVILEAYLRGCGTAMLHDFTQQVQVIEMLQKVTLDIKSLSAEKYDVSSQVISQLKQKLENLQNSQLPESFRVPYDPGLKAGALAIEKCKVMASKKKPLWLEFKCADPTALSNETIGIIFKHGDDLRQDMLILQILRIMESIWETESLDLCLLPYGCISTGDKIGMIEIVKDATTIAKIQQSTVGNTGAFKDEVLNHWLKEKSPTEEKFQAAVERFVYSCAGYCVATFVLGIGDRHNDNIMITETGNLFHIDFGHILGNYKSFLGINKERVPFVLTPDFLFVMGTSGKKTSPHFQKFQDICVKAYLALRHHTNLLIILFSMMLMTGMPQLTSKEDIEYIRDALTVGKNEEDAKKYFLDQIEVCRDKGWTVQFNWFLHLVLGIKQGEKHSA

SEQ ID NO: 5 - DNA, 20 bp, Artificial sequence
  synthetic
gctgtgcaggctgctctaac

SEQ ID NO: 6 - DNA, 20 bp, Artificial sequence
  synthetic
cgcatgatctgcatggtgat

(19) United States
(12) Patent Application Publication
(10) Pub. No.: US 2010/0278837 A1
(43) Pub. Date: Nov. 4, 2010

(54) Compositions And Methods For Reducing Cancer And Inflammation

(75) Inventors: Judith A. Varner, Encinitas, CA (US); David A. Cheresh, Encinitas, CA (US); Michael C. Schmid, Del Mar, CA (US)

Correspondence Address: Peter G. Carroll;MEDLEN & CARROLL, LLP, 101 Howard Street, Suite 350, San Francisco, CA, 94105, US

(73) Assignee: The Regents of the University of California

(21) Appl. No.: 12/720,348

(22) Filed: Mar. 9, 2010

Related U.S. Application Data
(60) Provisional application No. 61/158,482, filed on Mar. 9, 2009.

(51) Int. Cl. A61K 39/395; A61K 31/713; A61P 35/00

(52) U.S. Cl. 424/158.1; 514/44 A; 514/44 R

(57) ABSTRACT

This invention relates to the discovery of the convergence of diverse receptors and signaling pathways on the PI3gamma dependent activation of VLA4 (integrin a4b1). In particular, the invention relates to the role of myeloid cells in tumor inflammation and metastasis. The invention provides methods for inhibiting cancer in a subject comprising administering to a subject having cancer that comprises endothelial cells a therapeutically effective amount of a PI-3-kinase gamma inhibitor that reduces at least one of (a) adhesion of myeloid cells to the endothelial cells, (b) migration of myeloid cells into the cancer, (c) growth of the cancer, (d) activation of integrin a4b1 that is comprised on the myeloid cells, and (e) clustering of integrin a4b1 that is comprised on the myeloid cells.

This application claims priority to co-pending U.S. provisional Application Ser. No. 61/158,482, filed Mar. 9, 2009, herein incorporated by reference in its entirety for all purposes.

This invention was made, in part, with government support under grant numbers CA045726, CA050286, CA083133, CA098048, AR27214, HL31950, and R01CA118182 awarded by the National Cancer Institute of the National Institutes of Health. The government has certain rights in the invention.

### SUMMARY OF THE INVENTION

This invention relates to the discovery of the convergence of diverse receptors and signaling pathways on the PI3gamma dependent activation of VLA4 (integrin a4b1). In particular, the invention relates to the role of myeloid cells in tumor inflammation and metastasis. The invention provides methods for inhibiting cancer in a subject comprising administering to a subject having cancer that comprises endothelial cells a therapeutically effective amount of a PI-3-kinase gamma inhibitor that reduces at least one of (a) adhesion of myeloid cells to the endothelial cells, (b) migration of myeloid cells into the cancer, (c) growth of the cancer, (d) activation of integrin a4b1 that is comprised on the myeloid cells, and (e) clustering of integrin a4b1 that is comprised on the myeloid cells.

In one embodiment, the PI-3-kinase gamma inhibitor comprises an antibody that specifically binds to PI-3-kinase gamma. In an alternative embodiment, the PI-3-kinase gamma inhibitor comprises a nucleic acid sequence selected from PI-3-kinase gamma antisense sequence and PI-3-kinase gamma ribozyme sequence.

In one embodiment, the present invention contemplates siRNA mediated knockdown of p110γ to suppressed myeloid cell adhesion in a subject, such as a patient. In another embodiment, the present invention contemplates administering selective inhibitors of PI3-kinase γ, but not of other isoforms, to suppress chemoattractant-stimulated PI3-kinase catalytic activity and inhibit myeloid cell adhesion.

### BRIEF DESCRIPTION OF THE DRAWINGS

FIG. 1. Tumour derived and myeloid cell derived inflammatory factors are associated with CD11b+ myeloid cell recruitment. (a) Micrograph: Cryosections of murine tumours as well as corresponding normal tissue were immunostained to detect CD11b+ cells (red, arrowheads) and counterstained with DAPI (blue). Calibration bar indicates 40 μm. Graph: Mean CD11b+ pixels/field for normal (white bars) and tumour (black bars) tissue (n=6). (b) FACs profiles of CD11b+ cells from 0-21 day LLC tumours (n=6). (c) Percentage of CD11b+ cells and CD31+ pixels/field in 0-21 day LLC tumours (n=6). (d-e) qPCR analysis of gene expression for SDF-1α, IL-1β, IL-6, TNFα and VEGF-A in (d) cultured LLC cells (black bars, n=3) and LLC tumours (white bars, n=4), (n.d. indicates not detected) and in (e) CD11b− (white bars) and CD11b+ cells (black bars) isolated from LLC tumours (n=4). (f) Protein levels of SDF-1α, (black bars) and IL-1β (white bars) in LLC conditioned media determined by ELISA. (g) SDF-1α (black bars) and IL-1β (white bars) in LLC tumours and in CD11b+ and CD11b− cells isolated from LLC tumours (n=3). “N.d.” indicates not detected. Error bars indicate standard error of the mean (SEM). Statistical significance was evaluated using Student's two-tailed t-test.

FIG. 2. PI3kinase-γ dependent integrin α4β1 activation and clustering promotes myeloid cell adhesion. (a) Adhesion of fluorescently labeled human CD11b+ cells to endothelium in the presence of basal medium, SDF-1α, IL-1β, IL-6, IL-8 or VEGF (black), anti-α4β1 antibody (orange) or isotype-matched IgG (white) (n=3). (b) Adhesion to endothelium of fluorescently labeled murine wildtype CD11b+ cells, wildtype cells treated with medium or Pertussis toxin (Ptx) and MyD88−/− cells after IL-1β (black bars) or SDF-1 (white bars) stimulation. (c) Adhesion of fluorescent wildtype CD11b+ cells (WT) treated with medium, 10 μM TG020 (panPI3Kinase inhibitor), PI3K75 (PI3kinase α inhibitor) or TG115 (PI3kinase γ/δ inhibitor), and adhesion of PI3kinase γ−/− CD11b+ cells to endothelium in the presence of basal medium (black), IL-1β (gray), SDF-1α (light gray), IL-6 (white), IL-8 (light orange), and VEGF (dark orange). (d) FACs profile of HUTS21 antibody (which detects activated β1 integrin) and P4C10 antibody (which detects total β1 integrin) binding to unstimulated (grey filled) or SDF-1α, IL-1β, IL-6, IL-8 and Mn2+ stimulated (black line) human CD11b+ cells (n=3). (e) Micrograph: Clustering of integrin α4 (green, arrowheads) in human CD11b+ cells incubated with SDF-1α, IL-1β, or human serum albumin (HSA)-coated microspheres, with counterstaining for DNA (blue). Graph: Percentage of cells with clustered integrin α4 (n=150). (f) Micrograph: Integrin α4 (green) and CXCR4 (red) co-clustering in human CD11b+ cells incubated with SDF-1α, IL-1β, or HSA-coated microspheres (arrowheads). Graph: Percentage of integrin α4β1 co-clustered with CXCR4. (g) Percentage of CD11b+ cells with clustered integrin α4 after IL-1β (black) and SDF-1α (white) stimulation of wildtype cells treated with medium (Control), pertussis toxin (PTX), panPI3kinase inhibitor TG020, PI3kinase α inhibitor PI3K75, PI3kinase γ inhibitor TG115 and percentage of MyD88−/− CD11b+ cells with clustered integrin (n=150). Error bars indicate SEM. Statistical significance was determined by two tailed Student's t-test.

FIG. 3. Integrin α4β1 activation by PI3K γ is required for myeloid cell adhesion to endothelium and invasion. (a) Representative immunostaining for integrin α4 (green), paxillin (red) and DNA (blue) in WT and α4Y991A TCM stimulated CD11b+ cells. Co-localization of integrin and paxillin is indicated by arrowheads. (b) Quantification of the percent of integrin α4 co-localizing with paxillin in WT (black) and α4Y991A (white) CD11b+ cells incubated with TCM, SDF-1α, IL-1β-coated microspheres (n=100). (c) Percentage of WT (black) and α4Y991A (white) CD11b+ cells with clustered integrin α4 after incubation with basal medium or BSA-, SDF-1α, IL-1\tilde{β}, and TCM-coated microspheres (n=150). (d) Immunoprecipitates of integrin α4 from WT and α4Y991A (α4YA) BMDC with (+) or without (−) stimulation by TCM were electrophoresed and immunoblotted for integrin α4, talin, paxillin and immunoglobulin (IgG). Histogram: normalized ratios of talin and paxillin to integrin α4 by densitometry. (e) Adhesion of WT (black bars) and α4Y991A (white bars) CD11b+ cells to endothelial cell monolayers after treatment with basal medium, SDF-1α, IL-1β and TCM (n=3). (f) Migration of WT (black bars) and α4Y991A (white bars) CD11b+ cells on VCAM-1 or vitronectin coated transwells (n=4). Calibration bars indicate 5 μm. Error bars represent SEM. Statistical significance was determined by 4wo tailed Student's t-test.

FIG. 4. Reduced tumour inflammation, neovascularization and progression in mice with suppressed PI3kinase and integrin α4 functions. (a) Micrograph: Low power brightfield (left) and merged brightfield/fluorescent (right) confocal images of LLC tumours under dorsal skinfold chambers 60 min after adoptive transfer of green fluorescent WT and red fluorescent integrin α4Y991A CD11b+ cells (arrowheads) (n=9). Graph: CD11b+ cells per microscopic field. (b) Percent CD11b+ WT and α4Y991A cells accumulating in LLC tumours 24 h after transfer (n=6). (c-e) LLC cells were subcutaneously implanted for 14 days in WT and α4Y991A (n=6-8) mice. (c) Micrograph: Tumour cryosections immunostained for CD11b (red, arrowheads) and nuclei (blue). Graph: Percent CD11b+ cells in tumours as quantified by FACs. (d) CD31+ pixels/field in WT and α4Y991A tumours. (e) Mass of WT and α4Y991A tumours. (f-g) Mice subcutaneously implanted with LLC cells for 10 days were untreated or treated with PI3K γ/δ inhibitor TG115 or with an inert control. (f) Percent CD11b+ cells in tumours as quantified by FACs. (g) Mass of treated LLC tumours (n=10). (h-k) LLC tumours were grown from 0-21 days in animals transplanted with BM (n=6-8) as follows: WT host/WT BM (black), WT host/α4Y991A BM (orange), α4Y991A host/α4Y991A BM (red) and α4Y991A host/WT BM (gray). (h) Percent CD11b+Gr1+ cells in tumours. (i) Number of CD31+ pixels/field in tumour cryosections. (j) Mean tumour mass for each group. (k) qPCR analysis for expression of IL-1β in tumours from h. Bar indicates mean. Calibration bars for fluorescent images indicate 20 μm; for brightfield, 1 mm. Error bars represent SEM. Statistical significance was determined by two tailed Student's t-test.

FIG. 5: LLC tumour growth rate and IL-1β expression levels (a) Growth rate profile of 0-21 day LLC tumours (n=6). Corresponding tumour inflammation and vascularization rates are presented in FIG. 5c. (b) IL-1β expression was quantified by qPCR in CD11b+ cells isolated from bone marrow (BM) of non-tumour bearing animals (control), from BM of animals bearing 14 day old LLC tumours, and from 14 day old LLC tumours (n=3). Error bars indicate SEM. Statistical significance was determined by two tailed Student's t-test.

FIG. 6: Expression of inflammatory factors in Panc02 tumours. (a) Protein expression analysis for SDF-1α (black bars) and IL-1β (white bars) by ELISA in basal or conditioned medium from Panc02 in vitro cultured cells. IL-1β was not detected in cell culture supernatants (white bars) (n=3). (b-d) qPCR quantification for gene expression of (b) CD11b, (c) IL-1β and (d) SDF-1α during orthotopic pancreatic tumour development in wildtype (WT) animals. Results are expressed as fold increases in expression compared to those of normal pancreas. Error bars indicate SEM. Statistical significance was determined by two-tailed Student's t-test.

FIG. 7: SDF-1α and IL-1β promote BM derived cell recruitment to tumours and tumor angiogenesis. (a) Quantification of GFP+BM cells and (b) CD31+ blood vessel density in cryosections from EGFP-BM transplanted mice that were injected with SDF-1α, IL-1β and PBS saturated growth factor depleted Matrigel for 14 days. (n=6). Significance testing was performed by ANOVA coupled with posthoc Tukey's test for multiple pairwise comparison where *P<0.05 is considered to be significant for IL-1β and SDF-1α values compared to PBS. (c) Percentage of CD11b+ cells in LLC tumours after treatment for one week with function-blocking anti-IL1β or isotype-matched control antibodies. (d) Blood vessel density of anti-IL-1β treated tumours quantified by CD31+ immunohistochemical staining. (e) Tumour mass after anti-IL-1β treatment (n=12-14). (f) Percentage of CD11b+ cells in LLC tumours after treatment for one week with saline (n=6) or AMD3100. (g) Blood vessel density of AMD3100 treated tumours quantified by CD31+ immunohistochemical staining. (h) Tumour mass after AMD3100 treatment. (i) Tumour mass after combined anti-IL-1beta and AMD3100 treatment Error bars indicate SEM. Statistical significance was determined by two tailed Student's t-test.

FIG. 8: Inflammatory factors increase integrin α4β1 mediated CD11b+ cell adhesion. (a-c) Fluorescently labeled murine (a) and human (b,c) bone marrow derived CD11b+ cells were incubated on HUVEC monolayers (a) or (b-c) VCAM-1 coated culture plates in basal media or in basal media containing 200 ng/ml IL-1β, IL-6 or SDF-1α in the absence (black bars) or presence of an anti-α4 inhibitory antibody (orange bars) or isotype matched control IgG (white bars), n=3. (c) Fluorescently labeled human CD11b+ cells were incubated on VCAM-1 coated culture plates in basal media or in media containing 200 ng/ml SDF-1α in the absence (black bars) or presence of 25 μg/ml AMD3100 (gray bars), n=3. Error bars represent SEM. Statistical significance was determined by two tailed Student's t-test.

FIG. 9: Inflammatory factors induce murine CD11b+ cell integrin α4 clustering. Clustering of integrin α4 (green, arrowheads) in mouse CD11b+ cells incubated with SDF-1α, TCM or bovine serum albumin (BSA)-coated microspheres, with counterstaining for DNA (blue).

FIG. 10: Impaired integrin α4 clustering in α4Y991A CD11b+ cells. (a) Bone marrow derived cells isolated from WT and α4Y991A animals were analyzed for integrin α4 expression by flow cytometry. (b) Clustering of integrin α4 (green, arrowheads) and talin (red, arrowhead) in mouse CD11b+ cells incubated with SDF-1α coated microspheres. Cells were counterstained for DNA (blue). While wildtype CD11b+ cells exhibit integrin clustering and co-clustering with talin (yellow, merge), integrin α4Y991A CD11b+ fail to do so.

FIG. 11: Defective pancreatic tumour inflammation α4Y991 mice. (a) Mean 21 day LLC tumour weight in wildtype (WT) and α4Y991A mice (*P<0.05). (b-e) WT and α4Y991A (n=6-8) mice were implanted orthotopically with Panc02 cells for 30 days. (b) Tumor cryosections were immunostained for CD11b (red, arrowheads) and nuclei (blue). CD11b+ cells in tumors were quantified by immunohistochemistry. (c) CD31+ pixels/field in WT and α4Y991A tumors. (d) Mass of WT and α4Y991A tumors. Bar indicates mean. (e) Percent WT and α4Y991A mice with cytokeratin+Panc02 metastases to hilar lymph nodes. Calibration bars for fluorescent images indicate 20 μm; for brightfield, 1 mm. Error bars represent SEM. Statistical significance was determined by two tailed Student's t-test.

FIG. 12: Effect of PI3kinase inhibitors on tumor cell proliferation. LLC cells were cultured in the presence of DMSO, an inert control (placebo), TG115 (a PI3K γ/δ inhibitor) or TG020 (a pan-PI3K inhibitor) at 10 μM and 1 μM. Cell proliferation was assessed 24 h, 48 h, and 72 h later with an MTT assay kit. Results are presented as mean absorbance at 450 nm+/−SEM (n=4).

FIG. 13: Reduced Panc02 tumour growth and metastasis in α4Y991A animals. (a) Quantification of F4/80+ cells (as pixels/field) in 21 day LLC tumours from WT and α4Y991A BM transplanted animals. (b-f) Panc02 tumours were grown for 30 days in BM transplanted animals (n=6-7). (b) Quantification of CD11b+Gr1+ cells in tumours. (c) CD31+ pixels/field tumours. (d) Tumour mass in BM transplanted animals. (e) Haematoxylin and eosin staining of Panc02 metastases (white arrowheads) in diaphragm and colon in WT and α4Y991A animals. Normal tissue indicated by red arrowheads. Calibration bar indicates 40 μm. (f) Percentage of BM transplanted WT and α4YA mice with diaphragm, colon and kidney metastases. Error bars represent SEM. Statistical significance determined by ANOVA coupled with posthoc Tukey's test for multiple pairwise comparisons, where *P<0.05 is considered statistically significant.

FIG. 14: Quantification of CD11b+Gr1+ cells in spleen, peripheral blood and bone marrow. (a) EGFP+wildtype or α4YA BM cells in spleen of animals after adoptive transfer were quantified by flow cytometry (n=6). (b-d) LLC cells were implanted in WT (black circle) and α4Y991A (white square) mice and tumours were isolated at 0, 7 and 14 days after cell inoculation. Gr1+CD11b+ cells were quantified in blood and bone marrow at 0, 7 and 14 days by flow cytometry. (b) Percentage of Gr1+CD11b+ cells in peripheral blood over time. (c) Total Gr1+CD11b+ cells in peripheral blood over time (calculated by multiplying the percent Gr1+CD11b+ cells by the total number of mononuclear cells per ml blood). (d) Percentage of Gr1+CD11b+ cells in total bone marrow. Error bars indicate SEM. Statistical significance was determined by two tailed Student's t-test.

FIG. 15: Normal macrophage differentiation and angiogenic potential in α4YA mice. (a) Quantification of CD11b+ Gr1+ expression in in vitro differentiated WT and α4Y991A macrophages (MΦ). (b-c) Growth factor depleted Matrigel was admixed with no cells or with 1×10⁶ CD11b+ cells from WT or α4Y991A mice and implanted in mice for 10 days. (b) Quantification of CD31+ blood vessels in B (n=6). (c) Cryosections were immunostained to detect CD31+ blood vessels (green) and counterstained with DAPI (blue). Scale bar=20 μm. Error bars indicate SEM. Statistical significance was determined by ANOVA.

FIG. 16: Decreased tumour derived inflammatory cytokine expression levels in Y991A mice. (a) WT and α4Y991A C57B16 mice were transplanted with WT or α4Y991A (α4YA) bone marrow and subcutaneously inoculated with LLC cells, as in FIG. 11. After 21 days, tumours were excised and analyzed for IL-6, TNF-α, SDF-1α, and VEGF-A expression. Values represent fold increase compared to expression in saline-saturated growth factor depleted Matrigel plugs. Statistical significance was determined by ANOVA, P<0.05 was considered significant. (b-f) Normal pancreas (control) and 30 day orthotopic Panc02 tumours from WT and α4Y991A (YA) mice were analyzed by qPCR for expression of (b) CD11b, (c) IL-1b, (d) IL-6, (e) TNFα, (f) VEGF-A. Bars represent fold increase compared to normal pancreatic tissue. Error bars indicate SEM. (n=3). Statistical significance was determined by ANOVA, P<0.05 was considered significant

FIG. 17: Reduced bone marrow integrin α4 expression suppresses tumour growth. (a-c) Irradiated WT mice were reconstituted with bone marrow derived cells from Tie2Cre(+) α4 fl/fl or Tie2Cre(−) α4 fl/fl animals and subcutaneously implanted with LLC cells for 0, 14 or 21 days. (a) Integrin α4 expression on CD11b+ cells was quantified by FACs. Percentage of CD11b+α4+ cells in bone marrow is indicated. The CD11b+ cell population from Tie2− animals is 100% positive for integrin α4 expression, while the CD11b+ cell population from Tie2Cre+ cells is only 54% positive. (b) Percent Gr1+CD11b+ cells in LLC tumours over time in Tie2Cre(+) α4 fl/fl or Tie2Cre(−) α4 fl/fl animals was quantified by FACs from single cell isolates of tumours. (c) Average tumour weight (n=6). Error bars indicate SEM. *P<0.05. Significance testing was performed by ANOVA coupled with posthoc Tukey's test for multiple pairwise comparison where *P<0.05 is considered to be significant.

FIG. 18: Deletion of integrin in accelerates myeloid cell infiltration and tumour growth. (a-c) WT and CD11b−/− animals (n=10) were subcutaneously implanted with LLC cells for 14 days. (a) Tumour mass (b) Percent of Gr1+ cells in single cell isolates of tumours, as quantified by FACs. (c) Micrograph: Tumour cryosections immunostained for macrophage marker F4/80 (green, arrowheads) and nuclei (blue). Graph: Quantification of F4/80+ cells/field. Scale bar=20 μm. Error bars indicate SEM. Statistical significance was determined by two tailed Student's t-test.

FIG. 19: Inhibitors of Pikinase gamma but not alpha or beta integrin alpha 4 mediated adhesion to rsVCAM.

FIG. 20: PI3kinase gamma inhibitors block myeloid cell adhesion.

FIG. 21: Myeloid cell adhesion is PI3kinase gamma dependent.

FIG. 22: Myeloid cell chemoattractants rapidly activate myeloid cell PI3kinase.

FIG. 23: Decreased PI3kinase gamma activity in PI3 Kgamma −/− and inhibitor treated myeloid cells.

FIG. 24: Evaluation of tumor growth in PI3kinase gamma−/− mice.

FIG. 25: Quantification of tumor infiltrating myeloid cells.

FIG. 26: PI3kinase gamma inhibitors block tumor inflammation and growth.

FIG. 27. Integrin α4β1 activation by tumour-derived chemoattractants promotes myeloid cell adhesion to endothelium in vitro and in vivo (a) Left, CD11b+ pixels/field in normal human breast and invasive ductal breast carcinoma, normal mouse breast and PyMT breast carcinoma, normal mouse pancreas and orthotopic pancreatic carcinoma, mouse lung and orthotopic lung carcinoma and normal skin and s.c. lung carcinoma, (n=6-10), *P<0.001 vs normal tissue. Right, CD11b+ cells (red, arrowheads) and nuclei (blue) in normal human breast and invasive ductal carcinoma; scale bar, 40 μm. (b) Chemoattractant gene expression in normal lung (n=3), cultured LLC cells (n=3), LLC lung tumours (n=3), and CD11b− and CD11b+ cell populations from LLC lung tumours (n=4), *P<0.05 vs normal lung. (c) Adhesion of chemoattractant-treated myeloid cells (fluorescence units, F.U.) to EC in the absence (Control) or presence of control IgG (cIgG), anti-α4, anti-αM integrin and a small molecule inhibitor of integrin α4 (ELN476063) (n=3), *P<0.001 vs IgG. (d) Adhesion to EC of chemoattractant-treated WT, integrin α4Y991A, α4−/− and αM−/− myeloid cells, and α4 (Itga4) or αM (Itgam) siRNA treated myeloid cells (n=3), *P<0.001 vs WT. (e) VCAM-1/Fc binding to chemoattractant-treated WT or α4Y991A myeloid cells (mean fluorescence intensity, MFI) (n=3), *P<0.01 vs WT. (f) Number per 10⁵ LLC tumour cells of adoptively transferred myeloid cells from WT, α4Y991A, integrin α4−/−, or integrin αM−/− mice and α4 (Itga4) or αM (Itgam) siRNA treated myeloid cells in tumours from WT mice (n=3-6), *P<0.001 vs WT. Error bars indicate s.e.m.

FIG. 28. Integrin α4 activation required for tumour inflammation, neovascularization and progression. (a) Left, LLC, Panc02, B16 and PyMT tumour weight, CD11b+ pixels/field, and CD31+ pixels/field in WT and α4Y991A mice (n=6-8), *P<0.05 vs WT, **P<0.001. Middle, whole mounts of 9 week-old mammary glands from PyMT WT and α4Y991A animals (LN, lymph node; arrowhead, tumour). Right, percent area of normal tissue, hyperplasia, and carcinoma in these whole mounts (n=10). (b) Left, LLC or Panc02 tumour weight, percent Gr1+CD11b+ cells in tumour, and CD31+ pixels/field in animals transplanted with WT or α4Y991A (YA) BM, (n=8) *P<0.05 vs WT mice with WT BM. Right, percent WT or α4Y991A BM transplanted WT mice with Panc02 metastases in colon, diaphragm or kidney (n=8) *P<0.05 vs WT mice/WT BM. Images, H&E-stained diaphragm and colon from WT mice with WT BM or WT mice with YA BM. Metastases indicated by white arrowheads. Scale bar, 40 μm. (c) Tumour weight, CD11b+ pixels/field, and CD31+ pixels/field in cryosections from mice with subcutaneous LLC tumours or PyMT breast tumours treated with ELN476063 integrin α4 small molecule inhibitor or saline control, (n=10) *P<0.05, **P<0.001. vs control. Error bars indicate s.e.m.

FIG. 29. PI3-kinase γ-mediated activation of myeloid cell integrin α4β1 promotes tumour progression. (a) Relative gene expression levels of Pi3kα, Pi3kβ, Pi3kγ and Pi3kδ in murine CD11b+ myeloid cells. (b) Adhesion to VCAM-1 of chemoattractant-treated WT myeloid cells transfected with non-silencing, Pi3kα, Pi3kβ, Pi3kγ, or Pi3kδ siRNAs, p110γ−/− myeloid cells, myeloid cells treated with TG100-115, a PI3-kinase γ inhibitor (PI3Kγi-1), or an inactive control compound (Ctrl), (n=3), *P<0.001 vs WT. (c) Number per 10⁵ LLC tumour cells of adoptively transferred myeloid cells transfected with non-silencing, Pi3kα, Pi3kβ, Pi3kγ, or Pi3kδ siRNAs, myeloid cells from p110γ−/− mice, and myeloid cells pretreated without (Ctrl) or with PI3Kγi-1 (TG100-115) found in WT LLC tumours (n=3), *P<0.001 vs WT. (d) LLC tumour volume and weight (n=10) from mice that were treated for 3 weeks with control, 0, 0.05, 0.5 or 5 mg/ml daily doses of PI3Kγi-1 (TG100-115), or 5 mg/ml daily dose of PI3Kγi-2 (AS605240),*P<0.01, vs WT. (e) Tumour weight (n=10), percent Gr1+CD11b+ cells/tumour and percent CD31+ pixels/field (n=10) in LLC tumours grown in WT or p110γ−/− mice and in WT mice treated with 5 mg/kg PI3Kγi (TG100-115), *P<0.01 vs WT. (f) Total tumour burden (expressed as tumor weight) from control or PI3Kγ inhibitor-1 (TG100-115) treated FVB PyMT mice (n=10), *P<0.004 vs Control. (g) Images: H&E-stained and whole mount mammary glands from FVB PyMT+ female mice treated with PI3Kγi-1 (TG100-115) or control (Ctrl); scale bar, 40 μm (LN, lymph node; arrowhead, tumour). Graph: area of normal, hyperplastic and carcinoma tissue in whole mounts of treated mice (n=10). *P<0.001 carcinoma, *P<0.01 normal tissue and P=0.45 hyperplasia. (h) CD11b+ pixels/field and CD31+ pixels/field in treated breast tumors, *P<0.001 vs control. Error bars indicate s.e.m.

FIG. 30. PI3-kinase γ mediated integrin α4β1 activation depends on the small GTPases Ras and Rap1. (a) VCAM-1 adhesion of CD11b+ cells after siRNA-mediated knockdown of N, K and H-Ras or treatment with Ras selective farnesyltransferase inhibitor (FTi). *P<0.002 vs control group. (b) Adoptively transferred CD11b+ cells in LLC tumours after pretreatment of cells without (Control) or with FTi or after transfection with N+K-ras specific or non-silencing siRNAs (n=3) *P<0.01 vs control. (c) VCAM-1 adhesion of WT, PI3Kγ inhibitor-1 (TG100-115) treated, p110γ−/−, non-silencing, Pi3kγ, or integrin α4 siRNA transfected CD11b+ cells after control or RasV12 plasmid transfection (n=3) *P<0.001 vs vector control. (d) Immunoblot of GTP-Rap1 and total Rap1 in bone marrow derived cells treated with PI3Kγi (TG100-115) or inert control in the absence and presence of IL-1β, SDF-1α, or IL-6. (e) Adhesion to VCAM-1 of CD11b+ cells transfected with Rap1a siRNA or non-silencing siRNA or after treatment with GGTi (n=3) *P<0.002 vs control group. (f) Adoptively transferred CD11b+ cells in tumours after pretreatment with GGTi or non-silencing or Rap1a selective siRNAs (n=3) *P<0.01 vs control. (g) VCAM-1 adhesion of WT, PI3Kγ inhibitor-1 (TG100-115) treated, p110γ−/− and non-silencing, Pi3kγ or integrin α4 siRNA transfected CD11b+ cells after control or RapV12 plasmid transfection (n=3). *P<0.001 vs control. Error bars indicate s.e.m

FIG. 31: Model of Ras-PI3-kinase γ-Rap-mediated activation of myeloid cell integrin α4 and role in tumour progression. (a) In normal healthy tissues, myeloid cells, which are comprised of granulocytes and monocytes, pass through capillaries without arresting on endothelium. (b) In the tumour microenvironment, tumour or myeloid cell-derived chemoattractants stimulate myeloid cell adhesion to endothelium and extravasation. Once in the tumour, many of these cells differentiate into pro-angiogenic macrophages. (c) In normal tissues, quiescent endothelium does not express VCAM-1 or other receptors for myeloid cell adhesion, and integrin α4β1 on myeloid cells remains inactive. (d) In the tumour microenvironment, inflammatory factors such as IL-1β stimulate endothelial cell expression of VCAM-1. Tumour derived chemoattractants stimulate myeloid cell surface receptors to activate N- and K-Ras, thereby leading to the activation of PI3-kinase γ. PI3 kinase γ then activates Rap1. Rap1 promotes talin association with the cytoplasmic domain of the integrin β chain and paxillin (Pax) association with the integrin a chain, leading to a conformational change that activates α4β1. Integrin α4β1 then binds to its newly expressed counter-receptor on endothelium, VCAM-1, and promotes the adhesion of myeloid cells to endothelium.

FIG. 32: Characterization of CD11b+ cells in tumours. (a) Tissues were immunostained to detect CD11b+ cells (red, arrowheads) and nuclei (blue). Scale bars, 40 μm. (b) LLC tumours (d7-21) and normal tissue (d0) were immunostained to detect CD11b+ (red) myeloid cells and CD31+ endothelial cells (green). (c) Left, Quantification of CD11b+ and CD31+ cells by immunohistochemical (IHC) staining expressed as pixels/field or vessels/field. Scale bars, 40 μm. Right, fold increase in CD11b and CD31 gene expression over time as determined by qPCR. (n=3). *P<0.05. (d) Left: Orthotopic Panc02 tumours (d30) and normal pancreas (d0) immunostained for CD11b and CD31. Right: Fold increase in expression of CD11b and CD31 vs. normal pancreas by qPCR. (n=3), *P<0.04. Scale bars bars, 40 μm.

FIG. 33: Characterization of myeloid cell mobilization and recruitment to the tumour microenvironment. (a) Percentage of cells in LLC tumours expressing myeloid cell markers quantified by FACs, (n=3). (b) Percentage of F4/80, Ly6C, Ly6G, CD14, MHCII, c-kit or Tie2 positive Gr1lo/negCD11b+ and Gr1hiCD11b+ cells in tumours. (n=3). (c) Percentage of CD11b+ Gr1hi and CD11b+Gr1lo cells in BM, PB and tumours from naïve and d14 LLC tumour bearing mice. (d) Quantification of total Gr1+CD11b+ cells in bone marrow (BM), per μl of peripheral blood (PB) and in tumours over time in mice bearing LLC tumours. *P<0.05. (e) Hematological profile of peripheral blood from naïve and tumour bearing mice (percent of total WBCs).

FIG. 34: Gene and protein expression in LLC and Panc02 tumours in vivo (a) qPCR for chemoattractants in Panc02 cells in vitro (black bars, n=3) and Panc02 tumours in vivo (n=3). (b) qPCR for SDF-1α and IL-1β over time in subcutaneous LLC and orthotopic Panc02 tumours in vivo (n=3). *P<0.01. (c) SDF-1α and IL-1β protein expression detected in basal or conditioned medium from LLC or Panc02 cells, (n=3) *P<0.001. (d) SDF-1α and IL-1β protein expression detected in LLC tumours and in CD11b+ and CD11b− subpopulations isolated from 14 day LLC tumours (n=3). All error bars indicate s.e.m.

FIG. 35: SDF-1α and IL-1β promote BM derived cell recruitment to tumours and tumour growth. (a) EGFP+ cells and CD31+ blood vessel density in SDF-1α, IL-1β or saline saturated Matrigel implanted in EGFP BM transplanted mice (n=6) *P<0.05 (b) Percentage of CD11b+ cells in anti-IL-1β, SDF-1α antagonist (AMD3100) or control treated tumours. *P<0.003 (c) CD31+ blood vessel density in treated tumours. (d) Tumour mass in combined anti-IL-1β and AMD3100 treated tumours (n=12-14), *P<0.001. All error bars indicate s.e.m.

FIG. 36: Integrin α4 mediates chemoattractant stimulated myeloid cell adhesion (a) Adhesion of human CD11b+ cells to HUVEC monolayers in the absence or presence of anti-α4 inhibitory antibody or isotype matched control IgG (n=3) *P<0.01 vs. control (basal medium). (b) Adhesion of mouse CD11b+ cells isolated from normal mice to VCAM-1 in the absence (Control) or presence of isotype control (IgG), anti-α4 or anti-αM inhibitory antibodies or α4 small molecule inhibitor (ELN476063). (n=3) *P<0.01 vs. Control. (c) Adhesion to endothelial cells, VCAM-1, and ICAM-1 of sorted CD11b+ cell populations from BM of normal or LLC tumour-bearing mice upon stimulation with SDF-1α, IL-1β and IL-6, expressed as fluorescence units (F.U.). Populations included total Gr1+CD11b+ cells and Gr1lo/negCD11b+ and Gr1hiCD11b+ subpopulations. (d) Quantification of adoptively transferred, fluorescently labeled Gr1+CD11b+ cell populations isolated from BM of normal or tumour-bearing mice that are found in LLC tumours 2 hours after adoptive transfer (n=3). (e) Percent mouse CD11b+ cell adhesion to VCAM-1 stimulated by SDF-1α, IL-1β or tumour conditioned medium (TCM) in the presence of serial dilutions of ELN 476063, a small molecule inhibitor of integrin α4β1/α4β7. IC50s for SDF-1α and IL-1β=10 nM. (f) Adhesion to VCAM-1 of WT, α4Y991A, αM−/−, and α4−/− CD11b+ cells. Integrin α4−/− CD11b+ cells were isolated by FACs sorting from Tie2Cre+ [symbol] 4loxp/loxp mice. (n=3) *P<0.01 vs. WT. All error bars indicate s.e.m.

FIG. 37: Integrin α4 is critical for CD11b+ myeloid cell adhesion to endothelium/VCAM-1. (a) Validation of siRNA mediated knockdown of integrin α4 and αM in CD11b+ cells by qPCR (left) and flow cytometry (right). (b) Chemoattractant stimulated adhesion to VCAM-1 of itgα4 and itgαm siRNA transfected BM derived CD11b+ cells isolated from normal mice (n=3). *P<0.01 vs. Control. (c) Chemoattractant stimulated adhesion to endothelial cells of itgα4 and itgαm siRNA transfected BM derived CD11b+ cells isolated from LLC tumour bearing mice (n=3). *P<0.01 vs. Control. (d) Chemoattractant stimulated adhesion to VCAM-1 of itgα4 and itgαm siRNA transfected BM derived CD11b+ cells isolated from LLC tumour bearing mice (n=3). *P<0.01 vs. Control. (e) Fibronectin and VCAM-1 immunoblot of lysates from chemoattractant stimulated endothelial cells. (f) Left: Histogram of human CD11b+ cells stained with integrin β1 activation epitope recognizing antibody (HUTS21) in the absence (unstimulated) or presence (stimulated) of SDF-1α, IL1β, or Mn2+. Right: Mean fluorescence intensity of HUTS21 binding to CD11b+ cells incubated in various chemoattractants and the positive control activator, Mn2+. All error bars indicate s.e.m.

FIG. 38: Reduced activation of α4 integrin in α4Y991A CD11b+ cells (a) Immunoprecipitated integrin α4 from WT and α4Y991A BM with (+) or without (−) stimulation by LLC tumour conditioned medium (TCM) immunoblotted for integrin α4, talin, paxillin and immunoglobulin (IgG) (to demonstrate equal gel loading). (b) Ratio of paxillin/integrin α4 per condition as determined by densitometry. (c) Ratio of talin/integrin α4 per condition as determined by densitometry. (d, e) Gene expression of Il-1β, Il-6, Vegf-A, Sdf-1α, and Tnfα in (d) LLC tumours (d14) and (e) Panc02 tumours (d30) from WT and α4Y991A mice (n=3), *P<0.01 compared to WT. All error bars indicate s.e.m.

FIG. 39: Tumour inflammation and growth in αM−/− (CD11b−/−) mice. (a) LLC tumour weight in WT and integrin αM−/− mice (n=10) after in vivo. (b) Percent Gr1+ cells in tumours as quantified by FACs. (c) Left: F4/80 (green, arrowheads) and DAPI (blue) in WT and αM−/− tumours. Right: F4/80+ pixels/field. Scale bar, 40 μm. All error bars indicate s.e.m.

FIG. 40: Tumours in animals with α4Y991A BM exhibit reduced growth rate and reduced inflammation. (a) Cryosections of LLC tumours from BM transplanted animals immunostained to detect CD31 (green) or CD11b (red) and nuclei (blue) (n=6). (b) LLC tumour weights over time from BM transplanted animals. (c) Percent Gr1+CD11b+ cells in LLC tumours over time from BM transplanted animals. (d) CD31+ pixels/field in LLC tumours over time from BM transplanted animals. (e) Gene expression in d21 LLC tumours from BM transplanted animals expressed as percent of WT/WT BM values (n=3). *P<0.05; All error bars indicate s.e.m. Scale bar, 40 μm.

FIG. 41: Effect of PI3-kinase inhibitors on adhesion (a) Relative specificities of several PI3kinase inhibitors tested in in vitro adhesion assays. Shown are reported IC50 values in vitro kinase assays. Two PI3-kinase γ selective inhibitors, TG100-1151-2 and AS6052403 were used extensively in these studies. †Chemical structure of TG100-115¹⁻². (b) Titration of PI3-kinase inhibitors in SDF-1α and IL-1β induced CD11b+ myeloid cell adhesion to VCAM-1. (IC50 for TG100-115 for SDF-1α=158 nM, and for IL-1β=281 nM). TGX221 (PI3-kinase β selective inhibitor) and PI33Kalpha2 (PI3-kinase α selective inhibitor) had no effect on adhesion below 100 μM. (c) IC50 values for TG100-115 and AS605240 in in vitro and in vivo biological assays. (d) Pharmacokinetic parameters in Balb/c mice of TG100-115 after a single i.v. dose of 5 mg/kg. The half-life in mice in vivo is 0.22 hr and the concentration in serum at 1 hour is 7.1 ng/ml. (e) PI3-kinase activity assay in myeloid cells: Anti-pAkt, and anti-Akt immunoblots of lysates of WT CD11b+ cells that were stimulated for 3 min with SDF-1α or IL-1β in the absence (−) or presence (+) of 1 μM PI3Kγ i-1 inhibitor (TG100-115) and of lysates from SDF-1α or IL-1β stimulated p110γ −/− CD11b+ cells. (f) Peripheral blood mononuclear cells were isolated 1, 2, 4, 6 or 12 hours after i.v. injection of 5 mg/kg of PI3Kγ i-1 (TG100-115) or inactive control (Ctrl). Cells were stimulated for 3 minutes with SDF-1α, solubilized and lysates immunoblotted with anti-pAkt and anti-Akt. Data indicate that PI3kinase activity is inhibited for up to 12 hours after dosing. All error bars indicate s.e.m. References: 1) Doukas, J. et al. Phosphoinositide 3-Kinase©/™ inhibition limits infarct size after myocardial ischemia/reperfusion injury. Proc. Natl. Acad. Sci. USA. 103, 19866-19871 (2006). 2) Palanki M. S., et al. Discovery of 3,3′-(2,4-diaminopteridine-6,7-diyl)diphenol as an isozyme-selective inhibitor of PI3K for the treatment of ischemia reperfusion injury associated with myocardial infarction. Journal of Medicinal Chemistry 50, 4279-4294 (2007). 3) Camps, M. et al. Blockade of PI3-Kinase© suppresses joint inflammation and damage in mouse models of rheumatoid arthritis. Nature Medicine 11, 936-943 (2005). 4) Hayakawa, M. et al. Synthesis and biological evaluation of 4-morpholino-2-phenyl quinazolines and related derivatives as novel PI3-kinase p110 [symbol] inhibitors. Bioorg. Med. Chem. 14, 6847-6858 (2006). 5) Jackson, S. P. et al. PI3-kinase p110®: a new target for antithrombotic therapy. Nat. Med. 11, 507-514 (2005).

FIG. 42: PI3-kinase γ suppression inhibits integrin activation but not expression. (a, b) Validation of siRNA mediated knockdown of PI3K isoforms by (a) qPCR and (b) Western blotting. (c) Integrin α4 expression in BM cells from WT and p110 γ−/− (n=3). (d) Mean fluorescence intensity (MFI) of unstimulated or SDF-1α or IL-1β stimulated VCAM-1/FC ligand binding to murine CD11b+ myeloid cells determined by flow cytometry. WT cells were treated with 1 μM PI3 kinase γ inhibitors-1 (TG100-115) and -2 (AS605240), PI3-kinase [symbol] inhibitor (PI3Kalpha2) and with PI3-kinase β inhibitor (TGX221). VCAM binding to CD11b+ myeloid cells from p110γ−/− mice was also assessed. (n=3) *P<0.01 vs control. (e) HUTS21 antibody binding to unstimulated or SDF-1α stimulated human CD11b+ cells in the presence of no inhibitor (Ctrl), PI3kγi-1, PI3kαi or Mn2+ (n=3), *P<0.01 vs control. (f) CD11b+ cells isolated from BM of normal mice were incubated in basal medium or 1 μM PI3 kinase γ inhibitors-1 (TG100-115), PI3-kinase α inhibitor (PI3Kalpha2) or PI3-kinase β inhibitor (TGX221) fluorescently labeled and adoptively transferred into mice bearing in LLC tumours. Fluorescent cells present in tumours were quantified 2 hours after adoptive transfer (n=3), *P<0.001 vs Control. (g) Integrin α4 immunoprecipitates from WT and p110γ−/− BM incubated with (+) or without (−) TCM were immunoblotted for integrin α4, talin, paxillin and IgG. (h-i) Ratios of paxillin (h) and talin (i) to integrin α4 were determined by densitometry. All error bars indicate s.e.m.

FIG. 43: Bone marrow derived myeloid cells from normal and tumour bearing mice depend on PI3-kinase γ for adhesion and trafficking in vivo. (a,b) Chemoattractant stimulated adhesion to VCAM-1 of BM derived CD11b+ cells from (a) normal and (b) LLC (d14) tumour bearing mice after transfection with non silencing, Pi3kα, Pi3kβ, Pi3kγ or Pi3kδ siRNA (n=3) *P<0.01 vs. non silencing siRNA. (c) CD11b+ cells from normal mice (grey bars) or from LLC (d14) tumour bearing mice (black bars) were transfected with non silencing, Pi3kα, Pi3kβ, Pi3kγ or Pi3kδ siRNAs, fluorescently labeled and adoptively transferred into mice bearing 14 day old LLC tumours. Fluorescent cells present in tumours were quantified 2 hours after adoptive transfer (n=3), *P<0.01.

FIG. 44: Suppression of tumour growth in PI3-kinase γ −/− (p110γ−/−) mice (a) qPCR analysis of Il-1β, Il-6, Vegf-α, Sdf-α and Tnfα gene expression in LLC tumours from WT (white bars), p110γ−/− (grey bars) mice and from mice treated with 5 mg/kg PI3Kγi-1 (TG100-115) (black bars). (n=3), P<0.05. (b) Quantification of angiogenesis in bFGF saturated matrigel implanted in WT and p110γ−/− mice. Animals were injected with FITC-Lectin 15 minutes prior to sacrifice. Matrigel plugs were removed, digested with dispase and total fluorescence in the plugs was determined by fluorimetry (n=5). (c) Weight of B16 melanoma tumours grown in WT or p110γ−/− mice (n=10), *P<0.03. (d) Quantification of CD11b+ pixel density (left graph) and CD31+ pixel density in B16 tumours (right graph) *P<0.01. All error bars indicate s.e.m.

FIG. 45: Suppression of spontaneous breast tumour growth in PI3-kinase γ inhibitor treated mice. (a) F4/80+ macrophages in mammary glands of normal FVB female mice, PyMT+FVB female mice, and PyMT+FVB female mice treated with control or PI3Kγ i-1 (TG100-115). (b) F4/80 pixels/field (n=10), *P<0.01. All error bars indicate s.e.m.

FIG. 46: Effect of PI3-kinase inhibitors on tumor cell proliferation. In vitro proliferation of PyMT+ and LLC tumour cells after 24 h in the presence of DMSO (Control), 0, 0.1, 1 or 10 μM PI3Kγi-1 (TG100-115) or a pan-PI3-kinase inhibitor (n=4). Similar results were obtained after 48 h and 72 h (data not shown), (n=4) #P<0.02, *P<0.001. Error bars indicate s.e.m.

FIG. 47: Characterization of myeloid cells in [symbol] 4Y991A and p110γ−/− mice. Quantification of F4/80, Ly6C, Ly6G, CD14, MHCII, c-kit and Tie2 expression in Gr1lo/neg CD11b+ cells in LLC tumours in WT, [symbol] 4Y991A and p110γ−/− mice. No significant differences were observed in the proportion of various myeloid cell subpopulations in tumours in the three strains of mice, although significant differences were observed in the absolute numbers of Gr1lo/neg CD11b+ cells in tumours. *P<0.05.

FIG. 48: N- and K-Ras regulation of integrin α4 activation. (a) Lysates of Non silencing, H-ras and N+K-Ras siRNA transfected CD11b+ myeloid cells were immunoblotted to detect Ras and actin. (b) Mean fluorescence intensity (MFI) of unstimulated, SDF-1α or IL-1β stimulated VCAM-1/FC ligand binding to murine CD11b+ myeloid cells determined by flow cytometry. CD11b+ cells were pretreated with medium, 10 μM farnesyltransferase inhibitor (FTi) or 10 μM PLC inhibitor (PLCi) (n=3). *P<0.01 vs control. (c) HUTS21 antibody binding to SDF-1α stimulated human CD11b+ cells treated with medium, FTi, PLCi or Mn2+ (n=3). *P<0.01 vs control. (d) Mean fluorescence intensity (MFI) of VCAM-1/FC binding to control or RasV12 transfected WT and p110γ−/− CD11b+ cells (n=3). *P<0.001 vs vector control.

FIG. 49: Rap1 regulation of integrin α4 activation (a) Lysates of Non-silencing and Rap1 siRNA transfected CD11b+ myeloid cells were immunoblotted to detect Rap1 and actin. (b) Mean fluorescence intensity (MFI) of unstimulated, SDF-1α or IL-1β stimulated VCAM-1/FC ligand binding to murine CD11b+ myeloid cells determined by flow cytometry. CD11b+ cells were pretreated with medium, 10 μM geranylgeranyltransferase inhibitor (GGTi) or 10 μM PLC inhibitor (PLCi) (n=3) *P<0.002 vs control. (c) HUTS21 antibody binding to SDF-1α stimulated human CD11b+ cells treated with medium, 10 μM GGTi, 10 μM PLCi or Mn2+ (n=3). *P<0.002 vs control. (d) Mean fluorescence intensity (MFI) of VCAM-1/FC binding to WT and p110γ−/− CD11b+ cells after control or RapV12 plasmid transfection (n=3). *P<0.001 vs vector control.

FIG. 50: Mouse p110 gamma (Pik3cg) mRNA sequence (SEQ ID NO:1) and encoded amino acid sequence (SEQ ID NO:2) (GenBank Accession No. NM_{—}001146200) for mouse p110 gamma (Pik3cg) Gene ID: 30955.

FIG. 51: human PIK3CG mRNA sequence (SEQ ID NO:3) and encoded amino acid sequence (SEQ ID NO:4) (GenBank Accession No. NM_{—}002649.2) for human PIK3CG Gene ID: 5294.

### DEFINITIONS

To facilitate understanding of the invention, a number of terms are defined below.

As used in this specification and the appended claims, the singular forms “a,” “an” and “the” includes both singular and plural references unless the content clearly dictates otherwise.

As used herein, the term “or” when used in the expression “A or B,” where A and B refer to a composition, disease, product, etc., means one, or the other, or both.

The term “on” when in reference to the location of a first article with respect to a second article means that the first article is on top and/or into the second article, including, for example, where the first article permeates into the second article after initially being placed on it.

As used herein, the term “comprising” when placed before the recitation of steps in a method means that the method encompasses one or more steps that are additional to those expressly recited, and that the additional one or more steps may be performed before, between, and/or after the recited steps. For example, a method comprising steps a, b, and c encompasses a method of steps a, b, x, and c, a method of steps a, b, c, and x, as well as a method of steps x, a, b, and c. Furthermore, the term “comprising” when placed before the recitation of steps in a method does not (although it may) require sequential performance of the listed steps, unless the content clearly dictates otherwise. For example, a method comprising steps a, b, and c encompasses, for example, a method of performing steps in the order of steps a, c, and b, the order of steps c, b, and a, and the order of steps c, a, and b, etc

Unless otherwise indicated, all numbers expressing quantities of ingredients, properties such as molecular weight, reaction conditions, and so forth as used in the specification and claims are to be understood as being modified in all instances by the term “about.” Accordingly, unless indicated to the contrary, the numerical parameters in the specification and claims are approximations that may vary depending upon the desired properties sought to be obtained by the present invention. At the very least, and without limiting the application of the doctrine of equivalents to the scope of the claims, each numerical parameter should at least be construed in light of the number of reported significant digits and by applying ordinary rounding techniques. Notwithstanding that the numerical ranges and parameters describing the broad scope of the invention are approximation, the numerical values in the specific examples are reported as precisely as possible. Any numerical value, however, inherently contains standard deviations that necessarily result from the errors found in the numerical value's testing measurements.

The term “not” when preceding, and made in reference to, any particularly named molecule (such as a protein, nucleotide sequence, etc.) or phenomenon (such as cell adhesion, cell migration, cell differentiation, angiogenesis, biological activity, biochemical activity, etc.) means that only the particularly named molecule or phenomenon is excluded.

The term “altering” and grammatical equivalents as used herein in reference to the level of any molecule (such as a protein, nucleotide sequence, etc.) or phenomenon (such as cell adhesion, cell migration, cell differentiation, angiogenesis, biological activity, biochemical activity, etc.) refers to an increase and/or decrease in the quantity of the substance and/or phenomenon, regardless of whether the quantity is determined objectively and/or subjectively.

The term “increase,” “elevate,” “raise,” and grammatical equivalents when in reference to the level of a molecule (such as a protein, nucleotide sequence, etc.) or phenomenon (such as cell adhesion, cell migration, cell differentiation, angiogenesis, biological activity, biochemical activity, etc.) in a first sample relative to a second sample, mean that the quantity of the substance and/or phenomenon in the first sample is higher than in the second sample by any amount that is statistically significant using any art-accepted statistical method of analysis such as the Student's t-test. In one embodiment, the increase may be determined subjectively, for example when a patient refers to their subjective perception of disease symptoms, such as pain, clarity of vision, etc. In another embodiment, the quantity of the substance and/or phenomenon in the first sample is at least 10% greater than, preferably at least 25% greater than, more preferably at least 50% greater than, yet more preferably at least 75% greater than, and most preferably at least 90% greater than the quantity of the same substance and/or phenomenon in a second sample.

The terms “reduce,” “inhibit,” “diminish,” “suppress,” “decrease,” and grammatical equivalents when in reference to the level of a molecule (such as a protein, nucleotide sequence, etc.) or phenomenon (such as cell adhesion, cell migration, cell differentiation, angiogenesis, biological activity, biochemical activity, etc.) in a first sample relative to a second sample, mean that the quantity of substance and/or phenomenon in the first sample is lower than in the second sample by any amount that is statistically significant using any art-accepted statistical method of analysis. In one embodiment, the reduction may be determined subjectively, for example when a patient refers to their subjective perception of disease symptoms, such as pain, clarity of vision, etc. In another embodiment, the quantity of substance and/or phenomenon in the first sample is at least 10% lower than, preferably, at least 25% lower than, more preferably at least 50% lower than, yet more preferably at least 75% lower than, and most preferably at least 90% lower than the quantity of the same substance and/or phenomenon in a second sample. A reduced level of a molecule and/or phenomenon need not, although it may, mean an absolute absence of the molecule and/or phenomenon.

Reference herein to any specifically named protein (such as “integrin α4β1,” “vascular cell adhesion molecule,” fibronectin, PI-3-kinase gamma, etc.) refers to a polypeptide having at least one of the biological activities (such as those disclosed herein and/or known in the art) of the specifically named protein, wherein the biological activity is detectably by any method. In a preferred embodiment, the amino acid sequence of the polypeptide has at least 95% homology (i.e., identity) with the amino acid sequence of the specifically named protein. Reference herein to any specifically named protein (such as “integrin α4β1,” “vascular cell adhesion molecule,” fibronectin, PI-3-kinase gamma, etc.) also includes within its scope fragments, fusion proteins, and variants of the specifically named protein that have at least 95% homology with the amino acid sequence of the specifically named protein.

The term “fragment” when in reference to a protein refers to a portion of that protein that may range in size from four (4) contiguous amino acid residues to the entire amino acid sequence minus one amino acid residue. Thus, a polypeptide sequence comprising “at least a portion of an amino acid sequence” comprises from four (4) contiguous amino acid residues of the amino acid sequence to the entire amino acid sequence.

The term “variant” of a protein as used herein is defined as an amino acid sequence which differs by insertion, deletion, and/or conservative substitution of one or more amino acids from the protein. The term “conservative substitution” of an amino acid refers to the replacement of that amino acid with another amino acid which has a similar hydrophobicity, polarity, and/or structure. For example, the following aliphatic amino acids with neutral side chains may be conservatively substituted one for the other: glycine, alanine, valine, leucine, isoleucine, serine, and threonine. Aromatic amino acids with neutral side chains which may be conservatively substituted one for the other include phenylalanine, tyrosine, and tryptophan. Cysteine and methionine are sulphur-containing amino acids which may be conservatively substituted one for the other. Also, asparagine may be conservatively substituted for glutamine, and vice versa, since both amino acids are amides of dicarboxylic amino acids. In addition, aspartic acid (aspartate) my be conservatively substituted for glutamic acid (glutamate) as both are acidic, charged (hydrophilic) amino acids. Also, lysine, arginine, and histidine my be conservatively substituted one for the other since each is a basic, charged (hydrophilic) amino acid. Guidance in determining which and how many amino acid residues may be substituted, inserted or deleted without abolishing biological and/or immunological activity may be found using computer programs well known in the art, for example, DNAStar™ software. In one embodiment, the sequence of the variant has at least 95% identity, preferably at least 90% identity, more preferably at least 85% identity, yet more preferably at least 75% identity, even more preferably at least 70% identity, and also more preferably at least 65% identity with the sequence of the protein in issue.

Reference herein to any specifically named nucleotide sequence (such as a sequence encoding PI-3-kinase gamma, integrin α4β1, etc.) includes within its scope fragments, homologs, and sequences that hybridize under high and/or medium stringent conditions to the specifically named nucleotide sequence, and that have at least one of the biological activities (such as those disclosed herein and/or known in the art) of the specifically named nucleotide sequence, wherein the biological activity is detectable by any method.

The nucleotide “fragment” may range in size from an exemplary 10, 20, 50, 100 contiguous nucleotide residues to the entire nucleic acid sequence minus one nucleic acid residue. Thus, a nucleic acid sequence comprising “at least a portion of” a nucleotide sequence comprises from ten (10) contiguous nucleotide residues of the nucleotide sequence to the entire nucleotide sequence.

The term “homolog” of a specifically named nucleotide sequence refers to an oligonucleotide sequence which has at least 95% identity, more preferably at least 90% identity, yet more preferably at least 85% identity, yet more preferably at least 80% identity, also more preferably at least 75% identity, yet more preferably at least 70% identity, and most preferably at least 65% identity with the sequence of the nucleotide sequence in issue.

As used herein, the term “tissue exhibiting angiogenesis” refers to a tissue in which new blood vessels are developing from pre-existing blood vessels. The level of angiogenesis may be determined using methods well known in the art, including, without limitation, counting the number of blood vessels and/or the number of blood vessel branch points, as discussed herein. An alternative assay involves an in vitro cell adhesion assay that shows whether a compound inhibits the ability of α4β1-expressing cells (e.g. M21 melanoma cells) to adhere to VCAM or fibronectin. Another in vitro assay contemplated includes the tubular cord formation assay that shows growth of new blood vessels at the cellular level (D. S. Grant et al., Cell, 58: 933-943 (1989)). Art-accepted in vivo assays are also known, and involve the use of various test animals such as chickens, rats, mice, rabbits and the like. These in vivo assays include the chicken chorioallantoic membrane (CAM) assay, which is suitable for showing anti-angiogenic activity in both normal and neoplastic tissues (D. H. Ausprunk, Amer. J. Path., 79, No. 3: 597-610 (1975) and L. Ossonowski and E. Reich, Cancer Res., 30: 2300-2309 (1980)). Other in vivo assays include the mouse metastasis assay, which shows the ability of a compound to reduce the rate of growth of transplanted tumors in certain mice, or to inhibit the formation of tumors or pre-neoplastic cells in mice which are predisposed to cancer or which express chemically-induced cancer (M. J. Humphries et al., Science, 233: 467-470 (1986) and M. J. Humphries et al., J. Clin. Invest., 81: 782-790 (1988)).

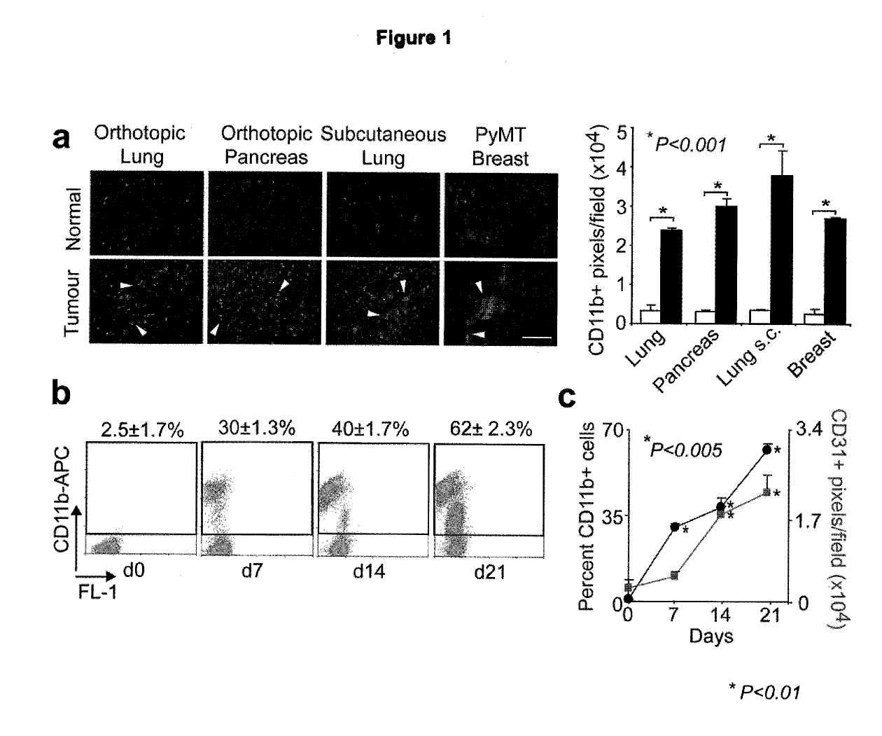

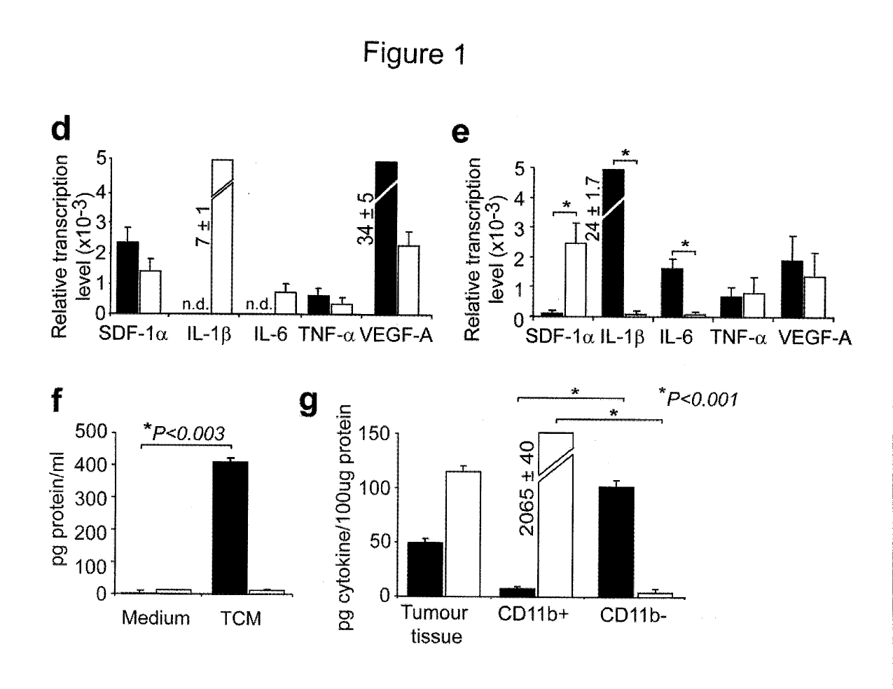

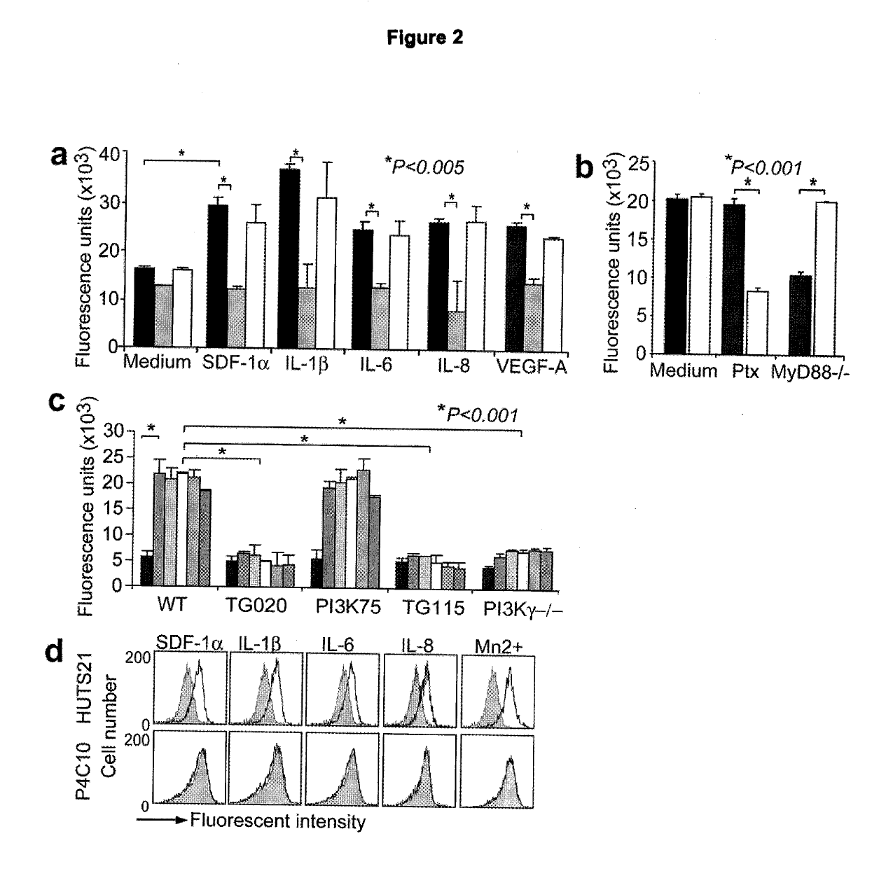

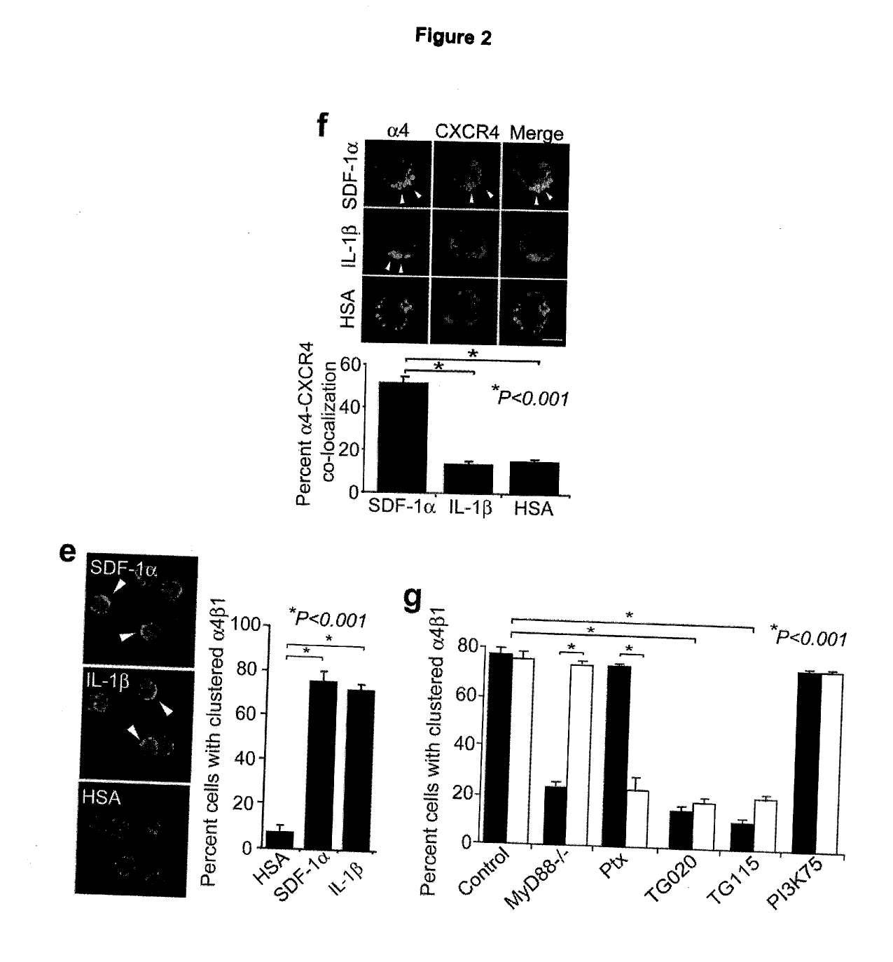

The term “integrin α4β1” is interchangeably used with the terms “CD49d/CD29,” “very late antigen 4,” and “VLA4” to refer to a member of the family of integrins. An “integrin” is an extracellular receptor that is expressed in a wide variety of cells and binds to specific ligands in the extracellular matrix. The specific ligands bound by integrins can contain an arginine-glycine-aspartic acid tripeptide (Arg-Gly-Asp; RGD) or a leucine-aspartic acid-valine (Leu-Asp-Val) tripeptide, and include, for example, fibronectin, vitronectin, osteopontin, tenascin, and von Willebrands's factor. Integrin α4β1 is a heterodimeric cell surface adhesion receptor composed of an α4 and β1 subunits that bind to ligands which are present in the extracellular matrix (ECM) as well as on the cell surface. An exemplary α4 polypeptide sequence is shown in FIG. 1, and an exemplary β1 polypeptide sequence is shown in FIG. 2.

The term “integrin α4β1” is contemplated also to include a portion of α4β1. The term “portion,” when used in reference to a protein (as in a “portion of α4β1”) refers to a fragment of that protein. The fragments may range in size from three (3) contiguous amino acid residues to the entire amino acid sequence minus one amino acid residue. Thus, a polypeptide sequence comprising “at least a portion of an amino acid sequence” comprises from three (3) contiguous amino acid residues of the amino acid sequence to the entire amino acid sequence.

The terms “isolated,” “purified,” and grammatical equivalents thereof when used in reference to a molecule (e.g., protein, DNA, RNA, etc.) or article (e.g., hematopoietic progenitor cell) in a sample refer to the reduction (by at least 10%, preferably by at least 25%, more preferably by at least 50%, even more preferably by at least 75%, and most preferably by at least 90%) in the amount of at least one contaminant molecule and/or article from the sample. Thus, purification results in an “enrichment,” i.e., an increase, in the amount of the desirable molecule and/or article relative to one or more other molecules and/or articles in the sample.

A “non-endothelial cell” is any cell type other than an endothelial cell (i.e., is not an endothelial cell) such as, without limitation, stem cell, lymph cell, mesenchymal cell, myeloid cell, lymphoid cell, granulocyte cell, macrophage cell, megakaryocyte cell, erythroid cell, B cell, T cell, bone marrow cell, muscle cell, neural cell, etc.

The terms “disease” and “pathological condition” are used interchangeably to refer to a state, signs, and/or symptoms that are associated with any impairment, interruption, cessation, or disorder of the normal state of a living animal or of any of its organs or tissues that interrupts or modifies the performance of normal functions, and may be a response to environmental factors (such as malnutrition, industrial hazards, or climate), to specific infective agents (such as worms, bacteria, or viruses), to inherent defect of the organism (such as various genetic anomalies, or to combinations of these and other factors. The term “disease” includes responses to injuries, especially if such responses are excessive, produce symptoms that excessively interfere with normal activities of an individual, and/or the tissue does not heal normally (where excessive is characterized as the degree of interference, or the length of the interference).

The term “adhesion” as used herein in reference to cells refers to the physical contacting of the cell to one or more components of the extracellular matrix (e.g., fibronectin, collagens I-XVIII, laminin, vitronectin, fibrinogen, osteopontin, Del 1, tenascin, von Willebrands's factor, etc.), to a ligand which is expressed on the cell surface (e.g., VCAM, ICAM, LI-CAM, VE-cadherin, integrin α2, integrin α3, etc.) and/or to another cell of the same type (e.g., adhesion of an HPC to another HPC) or of a different type (e.g., adhesion of an HPC to an endothelial cell, endothelial stem cell, stem cell expressing CD34, fibroblast cell, stromal cell, tumor cell, etc.).

The term “migration” as used herein in reference to cells refers to the translocation of a cell across one or more components of the extracellular matrix (e.g., fibronectin, collagens I-XVIII, laminin, vitronectin, fibrinogen, osteopontin, Del 1, tenascin, von Willebrands's factor, etc.), and/or along the surface of another cell of the same type (e.g., migration of an HPC along another HPC) and/or of a different cell (e.g., migration of an HPC along an endothelial cell, endothelial stem cell, stem cell expressing CD34, fibroblast cell, stromal cell, tumor cell, etc.). Thus, “trans-endothelial migration” of a cell refers to the translocation of the cell across one or more components of the extracellular matrix and/or cells of endothelial tissue.

In another embodiment, the subject has a neoplasm. The terms “neoplasm” and “tumor” refer to a tissue growth that is characterized, in part, by angiogenesis. Neoplasms may be benign and are exemplified, but not limited to, a hemangioma, glioma, teratoma, and the like. Neoplasms may alternatively be malignant, for example, a carcinoma, sarcoma, glioblastoma, astrocytoma, neuroblastoma, retinoblastoma, and the like.

The terms “malignant neoplasm” and “malignant tumor” refer to a neoplasm that contains at least one cancer cell. A “cancer cell” refers to a cell undergoing early, intermediate or advanced stages of multi-step neoplastic progression as previously described (H. C. Pitot (1978) in “Fundamentals of Oncology,” Marcel Dekker (Ed.), New York pp 15-28). The features of early, intermediate and advanced stages of neoplastic progression have been described using microscopy. Cancer cells at each of the three stages of neoplastic progression generally have abnormal karyotypes, including translocations, inversion, deletions, isochromosomes, monosomies, and extra chromosomes. A cell in the early stages of malignant progression is referred to as “hyperplastic cell” and is characterized by dividing without control and/or at a greater rate than a normal cell of the same cell type in the same tissue. Proliferation may be slow or rapid, but continues unabated. A cell in the intermediate stages of neoplastic progression is referred to as a “dysplastic cell.” A dysplastic cell resembles an immature epithelial cell, is generally spatially disorganized within the tissue and loses its specialized structures and functions. During the intermediate stages of neoplastic progression, an increasing percentage of the epithelium becomes composed of dysplastic cells. “Hyperplastic” and “dysplastic” cells are referred to as “pre-neoplastic” cells. In the advanced stages of neoplastic progression a dysplastic cell become a “neoplastic” cell. Neoplastic cells are typically invasive (i.e., they either invade adjacent tissues, or are shed from the primary site and circulate through the blood and lymph) to other locations in the body where they initiate one or more secondary cancers (i.e., “metastases”). Thus, the term “cancer” is used herein to refer to a malignant neoplasm, which may or may not be metastatic. Malignant neoplasms that can be diagnosed using a method of the invention include, for example, carcinomas such as lung cancer, breast cancer, prostate cancer, cervical cancer, pancreatic cancer, colon cancer, ovarian cancer; stomach cancer, esophageal cancer, mouth cancer, tongue cancer, gum cancer, skin cancer (e.g., melanoma, basal cell carcinoma, Kaposi's sarcoma, etc.), muscle cancer, heart cancer, liver cancer, bronchial cancer, cartilage cancer, bone cancer, testis cancer, kidney cancer, endometrium cancer, uterus cancer, bladder cancer, bone marrow cancer, lymphoma cancer, spleen cancer, thymus cancer, thyroid cancer, brain cancer, neuron cancer, mesothelioma, gall bladder cancer, ocular cancer (e.g., cancer of the cornea, cancer of uvea, cancer of the choroids, cancer of the macula, vitreous humor cancer, etc.), joint cancer (e.g., synovium cancer), glioblastoma, lymphoma, and leukemia. Malignant neoplasms are further exemplified by sarcomas (such as osteosarcoma and Kaposi's sarcoma). The invention expressly contemplates within its scope any malignant neoplasm, so long as the neoplasm is characterized, at least in part, by angiogenesis associated with α4β1 expression by the newly forming blood vessels.

The phrase “reduces at least one of a) adhesion of myeloid cells to said endothelial cells, b) migration of myeloid cells into said cancer, c) growth of said cancer, d) activation of integrin α4b1 that is comprised on said myeloid cells, and e) clustering of integrin α4b1 that is comprised on said myeloid cells” means that the amount (as measured in an assay) of a) adhesion, b) migration, c) growth, d) activation, and/or e) clustering is reduced as compared to the amount or level in the absence of treatment with the inhibitor. The effects of diminishing any one of these characteristics may be determined by methods routine to those skilled in the art including, but not limited to, angiography, ultrasonic evaluation, fluoroscopic imaging, fiber optic endoscopic examination, biopsy and histology, blood tests, imaging tests and the like which can be used to detect, by way of an example, a decrease in the growth rate or size of a neoplasm. Such clinical tests are selected based on the particular pathological condition being treated. For example, it is contemplated that the methods of the invention result in a “reduction in tumor tissue” (e.g., a decrease in the size, weight, and/or volume of the tumor tissue) as compared to a control tumor tissue (e.g., the same tumor prior to treatment with the invention's methods, or a different tumor in a control subject).

As used herein the terms “therapeutically effective amount” refers to an amount of the composition that delays, reduces, palliates, ameliorates, stabilizes, prevents and/or reverses one or more symptoms of a disease, such as cancer, compared to in the absence of the composition of interest. Examples with respect to cancer include, without limitation, tumor size, tumor number, incidence of metastases, etc. Specific “dosages” can be readily determined by clinical trials and depend, for example, on the route of administration, patient weight (e.g. milligrams of drug per kg body weight). As used herein, the actual amount, i.e., “dosage,” encompassed by the term “pharmaceutically effective amount,” “therapeutically effective amount” and “protective amount” will depend on the route of administration, the type of subject being treated, and the physical characteristics of the specific subject under consideration. These factors and their relationship to determining this amount are well known to skilled practitioners in the medical, veterinary, and other related arts. This amount and the method of administration can be tailored to achieve optimal efficacy but will depend on such factors as weight, diet, concurrent medication and other factors which those skilled in the art will recognize. The dosage amount and frequency are selected to create an effective level of the compound without substantially harmful effects.

### DESCRIPTION OF THE INVENTION

This invention relates to the discovery of the convergence of diverse receptors and signaling pathways on the PI3gamma dependent activation of VLA4 (integrin α4b1). Data herein shows the validity of the concept that tumor inflammation is a necessary precedent to angiogenesis. While specific inflammatory mediators help recruit myeloid cells to tumors and stem from structurally diverse receptors, all investigated here converge on this kinase-integrin and blocking the activation of this integrin (either directly or by antagonizing the kinase activation of it) was specific and effective.

Cancer and inflammation are linked, as chronic inflammatory diseases increase the risk of developing many tumour types¹, while growing tumours induce host inflammatory responses²⁻⁴. Tumours produce a multitude of inflammatory mediators that stimulate myeloid cell extravasation, resulting in tumour angiogenesis, growth and metastasis⁵⁻¹¹. Here, we show that a wide array of chemokines and cytokines activating structurally diverse receptors and signaling pathways in myeloid cells promote tumour inflammation, but surprisingly, each of these pathways converge on PI3kinase γ-dependent activation of VLA-4 (integrin α4β1). Chemoattractants released from tumour cells, such as SDF-1α, TNFα or VEGF, and those released from tumour-invading myeloid cells, such as IL-1β or IL-6, all stimulate PI3kinase γ-dependent integrin α4β1 activation and signaling with subsequent adhesion of myeloid cells to vascular endothelium. Mutations and inhibitors that impair activity or expression of VLA-4 or PI3kinase γ, but not β2 integrin, block myeloid cell responsiveness to chemoattractants, recruitment to tumours, tumour angiogenesis, growth and metastasis. Thus, regardless of the initiating event, VLA-4 activation by PI3kinase γ serves as a checkpoint in tumour inflammation. Antagonism of myeloid cell PI3kinase γ or activation of integrin α4β1 represents an innovative approach to control the malignant properties of tumours.

In particular, the invention relates to the role of myeloid cells in tumor inflammation and metastasis. Data herein shows the validity of the concept that tumor inflammation is a necessary precedent to angiogenesis. While specific inflammatory mediators help recruit myeloid cells to tumors and stem from structurally diverse receptors, all investigated here converge on this kinase-integrin and blocking the activation of this integrin (either directly or by antagonizing the kinase activation of it) was specific and effective. One of the advantages of the instant in invention is that PI3kinase gamma inhibitors are non-toxic, potent and have not been used previously to suppress tumor growth.

In one aspect, pharmacological and/or genetic inhibitors of PI-3-kinase gamma are used to block invasion of myeloid cells into tumors (inflammation) and thereby block tumor angiogenesis and tumor growth and metastasis. Data herein shows that PI3kinase gamma selective inhibitors block myeloid cell adhesion to endothelium in vitro and block myeloid cell invasion of tumors in vivo, with subsequent suppression of tumor growth. The data also show that PI3kinase activates integrin α4b1 function on myeloid cells, leading to increased cell adhesion and invasion of tumors. Data herein also show that 1) PI3kinase gamma inhibitors block myeloid cell adhesion to endothelium regardless of the stimulus, 2) PI3kinase gamma inhibitors block myeloid cell integrin activation and clustering, 3) These inhibitors block invasion of myeloid cells into tumors, and 4) These inhibitors block tumor growth. These data are supported by similar results in PI3kinase gamma null mice.

The following are exemplary PI-3-kinase gamma inhibitors that may be used in the invention.

1. Antibodies

In one embodiment, the PI-3-kinase gamma inhibitor is an antibody that specifically binds to PI-3-kinase gamma. The terms “antibody” and “immunoglobulin” are interchangeably used to refer to a glycoprotein or a portion thereof (including single chain antibodies), which is evoked in an animal by an immunogen and which demonstrates specificity to the immunogen, or, more specifically, to one or more epitopes contained in the immunogen. The term “antibody” expressly includes within its scope antigen binding fragments of such antibodies, including, for example, Fab, F(ab′)₂, Fd or Fv fragments of an antibody. The antibodies of the invention also include chimeric and humanized antibodies. Antibodies may be polyclonal or monoclonal. The term “polyclonal antibody” refers to an immunoglobulin produced from more than a single clone of plasma cells; in contrast “monoclonal antibody” refers to an immunoglobulin produced from a single clone of plasma cells. The term “specifically binds” refers to the fact that the antibody has higher affinity for the kinase then for other proteins (e.g. serum albumin, and the like) and will therefore display a stronger signal (e.g. in an in vitro assay) over background (e.g. at least 2 to 1, preferably more than 3:1, more preferably at least 5:1, still more preferably 10:1 over background).

Antibodies contemplated to be within the scope of the invention include naturally occurring antibodies as well as non-naturally occurring antibodies, including, for example, single chain antibodies, chimeric, bifunctional and humanized antibodies, as well as antigen-binding fragments thereof. Naturally occurring antibodies may be generated in any species including murine, rat, rabbit, hamster, human, and simian species using methods known in the art. Non-naturally occurring antibodies can be constructed using solid phase peptide synthesis, can be produced recombinantly or can be obtained, for example, by screening combinatorial libraries consisting of variable heavy chains and variable light chains as previously described (Huse et al., Science 246:1275-1281 (1989)). These and other methods of making, for example, chimeric, humanized, CDR-grafted, single chain, and bifunctional antibodies are well known to those skilled in the art (Winter and Harris, Immunol. Today 14:243-246 (1993); Ward et al., Nature 341:544-546 (1989); Hilyard et al., Protein Engineering: A practical approach (IRL Press 1992); and Borrabeck, Antibody Engineering, 2d ed. (Oxford University Press 1995).

As used herein, the term “antibody” when used in reference to an anti-PI-3-kinase gamma antibody, refers to an antibody which specifically binds to one or more epitopes on an PI-3-kinase gamma polypeptide or peptide portion thereof, and which may or may not include some or all of an RGD binding domain. In one embodiment, an anti-PI-3-kinase gamma antibody, or antigen binding fragment thereof, is characterized by having specific binding activity for PI-3-kinase gamma of at least about 1×10⁵M⁻¹, more preferably at least about 1×10⁶M⁻¹, and yet more preferably at least about 1×10⁷M⁻¹.

Those skilled in the art know how to make polyclonal and monoclonal antibodies that are specific to a desirable polypeptide. For example, monoclonal antibodies may be generated by immunizing an animal (e.g., mouse, rabbit, etc.) with a desired antigen and the spleen cells from the immunized animal are immortalized, commonly by fusion with a myeloma cell.

Immunization with antigen may be accomplished in the presence or absence of an adjuvant (e.g., Freund's adjuvant). Typically, for a mouse, 10 μg antigen in 50-200 μl adjuvant or aqueous solution is administered per mouse by subcutaneous, intraperitoneal or intra-muscular routes. Booster immunization may be given at intervals (e.g., 2-8 weeks). The final boost is given approximately 2-4 days prior to fusion and is generally given in aqueous form rather than in adjuvant.

Spleen cells from the immunized animals may be prepared by teasing the spleen through a sterile sieve into culture medium at room temperature, or by gently releasing the spleen cells into medium by pressure between the frosted ends of two sterile glass microscope slides. The cells are harvested by centrifugation (400×g for 5 min.), washed and counted.

Spleen cells are fused with myeloma cells to generate hybridoma cell lines. Several mouse myeloma cell lines which have been selected for sensitivity to hypoxanthine-aminopterin-thymidine (HAT) are commercially available and may be grown in, for example, Dulbecco's modified Eagle's medium (DMEM) (Gibco BRL) containing 10-15% fetal calf serum. Fusion of myeloma cells and spleen cells may be accomplished using polyethylene glycol (PEG) or by electrofusion using protocols that are routine in the art. Fused cells are distributed into 96-well plates followed by selection of fused cells by culture for 1-2 weeks in 0.1 ml DMEM containing 10-15% fetal calf serum and HAT. The supernatants are screened for antibody production using methods well known in the art. Hybridoma clones from wells containing cells that produce antibody are obtained (e.g., by limiting dilution). Cloned hybridoma cells (4−5×10⁶) are implanted intraperitoneally in recipient mice, preferably of a BALB/c genetic background. Sera and ascites fluids are typically collected from mice after 10-14 days.

The invention also contemplates humanized antibodies that are specific for at least a portion of PI-3-kinase gamma and/or its ligands. Humanized antibodies may be generated using methods known in the art, including those described in U.S. Pat. Nos. 5,545,806; 5,569,825 and 5,625,126, the entire contents of which are incorporated by reference. Such methods include, for example, generation of transgenic non-human animals which contain human immunoglobulin chain genes and which are capable of expressing these genes to produce a repertoire of antibodies of various isotypes encoded by the human immunoglobulin genes.

2. Nucleic Acid Sequences

In an alternative embodiment, the PI-3-kinase inhibitor is a nucleic acid sequence. The terms “nucleic acid sequence” and “nucleotide sequence” as used herein refer to two or more nucleotides that are covalently linked to each other. Included within this definition are oligonucleotides, polynucleotide, and fragments and/or portions thereof, DNA and/or RNA of genomic and/or synthetic origin which may be single- or double-stranded, and represent the sense or antisense strand. Nucleic acid sequences that are particularly useful in the instant invention include, without limitation, antisense sequences and ribozymes. The nucleic acid sequences are contemplated to bind to genomic DNA sequences or RNA sequences that encode PI-3-kinase gamma, thereby inhibiting the activity of PI-3-kinase gamma. Antisense and ribozyme sequences may be delivered to cells by transfecting the cell with a vector that expresses the antisense nucleic acid or the ribozyme as an mRNA molecule. Alternatively, delivery may be accomplished by entrapping ribozymes and antisense sequences in liposomes.

a. Antisense Sequences

Antisense sequences have been successfully used to inhibit the expression of several genes (Markus-Sekura (1988) Anal. Biochem. 172:289-295; Hambor et al. (1988) J. Exp. Med. 168:1237-1245; and patent EP 140 308), including the gene encoding VCAM1, one of the integrin α4β1 ligands (U.S. Pat. No. 6,252,043, incorporated in its entirety by reference). The terms “antisense DNA sequence” and “antisense sequence” as used herein interchangeably refer to a deoxyribonucleotide sequence whose sequence of deoxyribonucleotide residues is in reverse 5′ to 3′ orientation in relation to the sequence of deoxyribonucleotide residues in a sense strand of a DNA duplex. A “sense strand” of a DNA duplex refers to a strand in a DNA duplex that is transcribed by a cell in its natural state into a “sense mRNA.” Sense mRNA generally is ultimately translated into a polypeptide. Thus, an “antisense DNA sequence” is a sequence which has the same sequence as the non-coding strand in a DNA duplex, and which encodes an “antisense RNA” (i.e., a ribonucleotide sequence whose sequence is complementary to a “sense mRNA” sequence). The designation (−) (i.e., “negative”) is sometimes used in reference to the antisense strand, with the designation (+) sometimes used in reference to the sense (i.e., “positive”) strand. Antisense RNA may be produced by any method, including synthesis by splicing an antisense DNA sequence to a promoter that permits the synthesis of antisense RNA. The transcribed antisense RNA strand combines with natural mRNA produced by the cell to form duplexes. These duplexes then block either the further transcription of the mRNA or its translation, or promote its degradation.

Any antisense sequence is contemplated to be within the scope of this invention if it is capable of reducing the level of expression of PI-3-kinase gamma to a quantity which is less than the quantity of PI-3-kinase gamma expression in a control tissue which is (a) not treated with the antisense PI-3-kinase gamma sequence, (b) treated with a sense PI-3-kinase gamma sequence, or (c) treated with a nonsense sequence.

Antisense PI-3-kinase gamma sequences include, for example, sequences which are capable of hybridizing with at least a portion of PI-3-kinase gamma cDNA under high stringency or medium stringency conditions. Antisense PI-3-kinase gamma sequences may be designed using approaches known in the art. In a preferred embodiment, the antisense i PI-3-kinase gamma sequences are designed to be hybridizable to PI-3-kinase gamma mRNA that is encoded by the coding region of the PI-3-kinase gamma gene. Alternatively, antisense PI-3-kinase gamma sequences may be designed to reduce transcription by hybridizing to upstream nontranslated sequences, thereby preventing promoter binding to transcription factors.

In a preferred embodiment, the antisense oligonucleotide sequences of the invention range in size from about 8 to about 100 nucleotide residues. In yet a more preferred embodiment, the oligonucleotide sequences range in size from about 8 to about 30 nucleotide residues. In a most preferred embodiment, the antisense sequences have 20 nucleotide residues.

The antisense oligonucleotide sequences that are useful in the methods of the instant invention may comprise naturally occurring nucleotide residues as well as nucleotide analogs. Nucleotide analogs may include, for example, nucleotide residues that contain altered sugar moieties, altered inter-sugar linkages (e.g., substitution of the phosphodiester bonds of the oligonucleotide with sulfur-containing bonds, phosphorothioate bonds, alkyl phosphorothioate bonds, N-alkyl phosphoramidates, phosphorodithioates, alkyl phosphonates and short chain alkyl or cycloalkyl structures), or altered base units. Oligonucleotide analogs are desirable, for example, to increase the stability of the antisense oligonucleotide compositions under biologic conditions since natural phosphodiester bonds are not resistant to nuclease hydrolysis. Oligonucleotide analogs may also be desirable to improve incorporation efficiency of the oligonucleotides into liposomes, to enhance the ability of the compositions to penetrate into the cells where the nucleic acid sequence whose activity is to be modulated is located, in order to reduce the amount of antisense oligonucleotide needed for a therapeutic effect thereby also reducing the cost and possible side effects of treatment.

Antisense oligonucleotide sequences may be synthesized using any of a number of methods known in the art, as well as using commercially available services (e.g., Genta, Inc.). Synthesis of antisense oligonucleotides may be performed, for example, using a solid support and commercially available DNA synthesizers. Alternatively, antisense oligonucleotides may also be synthesized using standard phosphoramidate chemistry techniques. For example, it is known in the art that for the generation of phosphodiester linkages, the oxidation is mediated via iodine, while for the synthesis of phosphorothioates, the oxidation is mediated with 3H-1,2-benzodithiole-3-one,1-dioxide in acetonitrile for the step-wise thioation of the phosphite linkages. The thioation step is followed by a capping step, cleavage from the solid support, and purification on HPLC, e.g., on a PRP-1 column and gradient of acetonitrile in triethylammonium acetate, pH 7.0. In one embodiment, the antisense DNA sequence is an “PI-3-kinase gamma antisense DNA sequence” (i.e., an antisense DNA sequence which is designed to bind with at least a portion of the PI-3-kinase gamma genomic sequence or with PI-3-kinase gamma mRNA).

b. Ribozyme

In some alternative embodiments, the PI-3-kinase gamma inhibitor is a ribozyme. Ribozyme sequences have been successfully used to inhibit the expression of several genes including the gene encoding VCAM1, which is one of the integrin α4β1 ligands (U.S. Pat. No. 6,252,043, incorporated in its entirety by reference).

The term “ribozyme” refers to an RNA sequence that hybridizes to a complementary sequence in a substrate RNA and cleaves the substrate RNA in a sequence specific manner at a substrate cleavage site. Typically, a ribozyme contains a “catalytic region” flanked by two “binding regions.” The ribozyme binding regions hybridize to the substrate RNA, while the catalytic region cleaves the substrate RNA at a “substrate cleavage site” to yield a “cleaved RNA product.” The nucleotide sequence of the ribozyme binding regions may be completely complementary or partially complementary to the substrate RNA sequence with which the ribozyme binding regions hybridize. Complete complementarity is preferred, in order to increase the specificity, as well as the turnover rate (i.e., the rate of release of the ribozyme from the cleaved RNA product), of the ribozyme. Partial complementarity, while less preferred, may be used to design a ribozyme binding region containing more than about 10 nucleotides. While contemplated to be within the scope of the claimed invention, partial complementarity is generally less preferred than complete complementarity since a binding region having partial complementarity to a substrate RNA exhibits reduced specificity and turnover rate of the ribozyme when compared to the specificity and turnover rate of a ribozyme which contains a binding region having complete complementarity to the substrate RNA. A ribozyme may hybridize to a partially or completely complementary DNA sequence but cannot cleave the hybridized DNA sequence since ribozyme cleavage requires a 2′-OH on the target molecule, which is not available on DNA sequences.

The ability of a ribozyme to cleave at a substrate cleavage site may readily be determined using methods known in the art. These methods include, but are not limited to, the detection (e.g., by Northern blot analysis as described herein, reverse-transcription polymerase chain reaction (RT-PCR), in situ hybridization and the like) of reduced in vitro or in vivo levels of RNA which contains a ribozyme substrate cleavage site for which the ribozyme is specific, compared to the level of RNA in controls (e.g., in the absence of ribozyme, or in the presence of a ribozyme sequence which contains a mutation in one or both unpaired nucleotide sequences which renders the ribozyme incapable of cleaving a substrate RNA).

Ribozymes contemplated to be within the scope of this invention include, but are not restricted to, hammerhead ribozymes (See e.g., Reddy et al., U.S. Pat. No. 5,246,921; Taira et al., U.S. Pat. No. 5,500,357, Goldberg et al., U.S. Pat. No. 5,225,347, the contents of each of which are herein incorporated by reference), Group I intron ribozyme (Kruger et al. (1982) Cell 31: 147-157), ribonuclease P (Guerrier-Takada et al. (1983) Cell 35: 849-857), hairpin ribozyme (Hampel et al., U.S. Pat. No. 5,527,895 incorporated by reference), and hepatitis delta virus ribozyme (Wu et al. (1989) Science 243:652-655).

A ribozyme may be designed to cleave at a substrate cleavage site in any substrate RNA so long as the substrate RNA contains one or more substrate cleavage sequences, and the sequences flanking the substrate cleavage site are known. In effect, expression in vivo of such ribozymes and the resulting cleavage of RNA transcripts of a gene of interest reduces or ablates expression of the corresponding gene.

For example, where the ribozyme is a hammerhead ribozyme, the basic principle of a hammerhead ribozyme design involves selection of a region in the substrate RNA which contains a substrate cleavage sequence, creation of two stretches of antisense oligonucleotides (i.e., the binding regions) which hybridize to sequences flanking the substrate cleavage sequence, and placing a sequence which forms a hammerhead catalytic region between the two binding regions.

In order to select a region in the substrate RNA which contains candidate substrate cleavage sites, the sequence of the substrate RNA needs to be determined. The sequence of RNA encoded by a genomic sequence of interest is readily determined using methods known in the art. For example, the sequence of an RNA transcript may be arrived at either manually, or using available computer programs (e.g., GENEWORKS, from IntelliGenetic Inc., or RNADRAW available from the internet at ole@mango.mef.ki.se), by changing the T in the DNA sequence encoding the RNA transcript to a U.

Substrate cleavage sequences in the target RNA may be located by searching the RNA sequence using available computer programs. For example, where the ribozyme is a hammerhead ribozyme, it is known in the art that the catalytic region of the hammerhead ribozyme cleaves only at a substrate cleavage site which contains a NUH, where N is any nucleotide, U is a uridine, and H is a cytosine (C), uridine (U), or adenine (A) but not a guanine (G). The U-H doublet in the NUH cleavage site does not include a U-G doublet since a G would pair with the adjacent C in the ribozyme and prevent ribozyme cleavage. Typically, N is a G and H is a C. Consequently, GUC has been found to be the most efficient substrate cleavage site for hammerhead ribozymes, although ribozyme cleavage at CUC is also efficient.

In a preferred embodiment, the substrate cleavage sequence is located in a loop structure or in an unpaired region of the substrate RNA. Computer programs for the prediction of RNA secondary structure formation are known in the art and include, for example, “RNADRAW”, “RNAFOLD” (Hofacker et al. (1994) Monatshefte F. Chemie 125:167-188; McCaskill (1990) Biopolymers 29:1105-1119). “DNASIS” (Hitachi), and “THE VIENNA PACKAGE.”

In addition to the desirability of selecting substrate cleavage sequences which are located in a loop structure or an unpaired region of the substrate RNA, it is also desirable, though not required, that the substrate cleavage sequence be located downstream (i.e., at the 3′-end) of the translation start codon (AUG or GUG) such that the translated truncated polypeptide is not biologically functional.

In a preferred embodiment, the ribozyme is an “PI-3-kinase gamma ribozyme” (i.e., a ribozyme whose substrate cleavage sequence is designed to hybridize with a portion of PI-3-kinase gamma that is involved in the biological activity of PI-3-kinase gamma).

One of skill in the art appreciates that it is not necessary that the two binding regions that flank the ribozyme catalytic region be of equal length. Binding regions that contain any number of nucleotides are contemplated to be within the scope of this invention so long as the desirable specificity of the ribozyme for the RNA substrate and the desirable cleavage rate of the RNA substrate are achieved. One of skill in the art knows that binding regions of longer nucleotide sequence, while increasing the specificity for a particular substrate RNA sequence, may reduce the ability of the ribozyme to dissociate from the substrate RNA following cleavage to bind with another substrate RNA molecule, thus reducing the rate of cleavage. On the other hand, though binding regions with shorter nucleotide sequences may have a higher rate of dissociation and cleavage, specificity for a substrate cleavage site may be compromised.

It is well within the skill of the art to determine an optimal length for the binding regions of a ribozyme such that a desirable specificity and rate of cleavage are achieved. Both the specificity of a ribozyme for a substrate RNA and the rate of cleavage of a substrate RNA by a ribozyme may be determined by, for example, kinetic studies in combination with Northern blot analysis or nuclease protection assays.

In a preferred embodiment, the complementarity between the ribozyme binding regions and the substrate RNA is complete. However, the invention is not limited to ribozyme sequences in which the binding regions show complete complementarity with the substrate RNA. Complementarity may be partial, so long as the desired specificity of the ribozyme for a substrate cleavage site and the rate of cleavage of the substrate RNA are achieved. Thus, base changes may be made in one or both of the ribozyme binding regions as long as substantial base pairing with the substrate RNA in the regions flanking the substrate cleavage sequence is maintained and base pairing with the substrate cleavage sequence is minimized. The term “substantial base pairing” means that greater than about 65%, more preferably greater than about 75%, and yet more preferably greater than about 90% of the bases of the hybridized sequences are base-paired.

It may be desirable to increase the intracellular stability of ribozymes expressed by an expression vector. This is achieved by designing the expressed ribozyme such that it contains a secondary structure (e.g., stem-loop structures) within the ribozyme molecule. Secondary structures which are suitable for stabilizing ribozymes include, but are not limited to, stem-loop structures formed by intra-strand base pairs. An alternative to the use of a stem-loop structure to protect ribozymes against ribonuclease degradation is by the insertion of a stem loop at each end of the ribozyme sequence (Sioud and Drlica (1991) Proc. Natl. Acad. Sci. USA 88:7303-7307). Other secondary structures which are useful in reducing the susceptibility of a ribozyme to ribonuclease degradation include hairpin, bulge loop, interior loop, multibranched loop, and pseudoknot structure as described in “Molecular and Cellular Biology,” Stephen L. Wolfe (Ed.), Wadsworth Publishing Company (1993) p. 575. Additionally, circularization of the ribozyme molecule protects against ribonuclease degradation since exonuclease degradation is initiated at either the 5′-end or 3′-end of the RNA. Methods of expressing a circularized RNA are known in the art (see, e.g., Puttaraju et al. (1993) Nucl. Acids Res. 21:4253-4258).

Once a ribozyme with desirable binding regions, a catalytic region and nuclease stability has been designed, the ribozyme may be produced by any known means including chemical synthesis. Chemically synthesized ribozymes may be introduced into a cell by, for example, microinjection electroporation, lipofection, etc. In a preferred embodiment, ribozymes are produced by expression from an expression vector that contains a gene encoding the designed ribozyme sequence.

3. Administering Agents

An agent that is useful in inhibiting PI-3-kinase gamma may be administered by various routes including, for example, orally, intranasally, or parenterally, including intravenously, intramuscularly, subcutaneously, intraorbitally, intracapsularly, intrasynovially, intraperitoneally, intracisternally or by passive or facilitated absorption through the skin using, for example, a skin patch or transdermal iontophoresis. Furthermore, the agent can be administered by injection, intubation, via a suppository, orally or topically, the latter of which can be passive, for example, by direct application of an ointment or powder containing the agent, or active, for example, using a nasal spray or inhalant. The agent can also be administered as a topical spray, if desired, in which case one component of the composition is an appropriate propellant. The pharmaceutical composition also can be incorporated, if desired, into liposomes, microspheres or other polymer matrices (Gregoriadis, “Liposome Technology,” Vol. 1, CRC Press, Boca Raton, Fla. 1984). Liposomes, for example, which consist of phospholipids or other lipids, are nontoxic, physiologically acceptable and metabolizable carriers that are relatively simple to make and administer. Liposomes are lipid-containing vesicles having a lipid bilayer as well as other lipid carrier particles that can entrap chemical agents. Liposomes may be made of one or more phospholipids, optionally including other materials such as sterols. Suitable phospholipids include phosphatidyl cholines, phosphatidyl serines, and many others that are well known in the art. Liposomes can be unilamellar, multilamellar or have an undefined lamellar structure. For example, in an individual suffering from a metastatic carcinoma, the agent in a pharmaceutical composition can be administered intravenously, orally or by another method that distributes the agent systemically.

Agents that are PI-3kinase gamma inhibitors may be administered in conjunction with other therapies. For example, in the case of cancer therapy, the agent may be administered in conjunction with conventional drug therapy and/or chemotherapy that is directed against solid tumors and for control of establishment of metastases. In one embodiment, the agent is administered during or after chemotherapy. In a more preferred embodiment, the agent is administered after chemotherapy, at a time when the tumor tissue will be responding to the toxic assault. In an alternative embodiment, the agent may be administered after surgery in which solid tumors have been removed as a prophylaxis against future metastases.

The “subject” to whom the agents are administered includes any animal which is capable of developing cancer in a tissue, including, without limitation, human and non-human animals such simians, rodents, ovines, bovines, ruminants, lagomorphs, porcines, caprines, equines, canines, felines, ayes, etc. Preferred non-human animals are members of the Order Rodentia (e.g., mouse and rat). Thus, the compounds of the invention may be administered by human health professionals as well as veterinarians.

### EXPERIMENTAL

The following serves to illustrate certain preferred embodiments and aspects of the present invention and are not to be construed as limiting the scope thereof.

### Example 1

### Methods Used in Examples 2-8

### Tumour Studies

LLC cells were obtained from the American Type Culture Collection (ATCC) and Panc02 pancreatic ductal carcinoma cells were obtained from the NCI DCTDC Tumour Repository. The abdominal cavities of immunocompetent C57Bl6 mice and integrin α4Y991A knockin mice³⁰ were opened and the tails of the pancreas were exteriorized. One million Panc02 cells were injected into the pancreatic tail, the pancreas was placed back into the abdominal cavity and the incision was closed. Tumours were excised after 30 days. 5×10⁵ LLC cells were injected subcutaneously or by intravenous tail vein injection into C57Bl6 or integrin α4Y991A mice. Tumours were excised at 7, 14 or 21 days, cryopreserved in O.C.T., lysed for RNA purification or collagenase-digested for flow cytometric analysis of CD11b and Gr1 expression. In some studies, C57Bl6 mice were subcutaneously implanted with LLC cells and treated on d3 and d5 by intraperitoneal injection with 100 μg/25 g of function-blocking anti-IL1β (rat IgG1, MAB401 from R&D Systems) (n=16) or isotype-matched control antibodies (n=14). In alternative studies, mice were treated by i.p. injection with saline (n=6) or 1.25 mg/kg AMD3100 (Sigma Aldrich) (n=7) for eight days or with 2.5 mg/kg TG115 or inert control (n=10) twice daily for ten days after LLC cell implantation. Tumours were harvested, weighed and further analyzed on day 8.

Quantification of Myeloid Cells from Tissues

To quantify myeloid cells in tissues, tumours were excised, minced and digested to single cell suspensions for 2 h at 37° C. in 10 ml of Hanks Balanced Salt Solution (HBSS, GIBCO) containing 10 mg/ml Collagenase type IV (Sigma), 1 mg/ml Hyaluronidase type V (Sigma) and 200 units/ml DNase type IV (Sigma). Cells were incubated in FC-blocking reagent (BD Bioscience), followed by CD11b-APC (M1/70, eBioscience) and Gr1-FITC (RB6-8C5, eBioscience). To exclude dead cells, 2.5 μg/ml propidium iodide (PI) was added before data acquisition by FACs Calibur (BD Bioscience). To quantify myeloid cells in murine peripheral blood, blood was collected by retro-orbital bleeding into heparin-coated Vacutainer tubes (BD Bioscience), incubation in red blood cell lysis buffer and CD11b-APC/Gr1-FITC staining.

### Analysis of Integrin Activation and Clustering

Activation of human CD29 (integrin β1 chain) in cytokine stimulated human CD11b+ cells was quantified by flow cytometry using HUTS-21 antibodies (BD Bioscience). Total β1 integrin levels were assessed using P4C10 antibodies (Chemicon). 2.5×10⁶ freshly isolated human myeloid cells/ml in culture medium containing 5% FBS and 0.5% NaN₃ were incubated in 10 μg/ml normal human immunoglobulin (12000C, Caltag) for 45 min on ice, then in 2 μg/ml SDF-1α, IL-1β, IL-6 or 1 mM Mn2+ plus 2.5 μg HUTS21, P4C10, or IgG2 control for 10 min at 37° C. followed by Alexa 488 goat-anti mouse antibodies for 20 min on ice. Expression levels of murine integrin α4 on bone marrow derived cells were determined by flow cytometry for PE-conjugated R1/2 (rat anti-CD49d antibody, eBioscience). To induce integrin α4 activation on human myeloid cells or murine WT and α4Y991A myeloid, purified CD11b+ cells were incubated with polystyrene microspheres (9.0 μm diameter, Bangs Laboratories) coated with TCM, 2 μg/ml SDF-1α or IL-1β overnight at 4° C. cells as previously described by Grabovsky et al., 2000. Some cells were incubated in 10 μM of a pan-PI3kinase inhibitor TG020, PI3kinase γ/δ inhibitor TG115, and PI3kinase α inhibitor PI3K75 (the kind gift of Kevan Shokat, University of California, San Francisco) and an inert control. Cells were incubated with beads for 5 min at 37° C., then fixed in 1% paraformaldehyde. Cells were then incubated with 5% BSA and FC-Blocker (BD Bioscience) for 30 min at room temperature followed by FITC-conjugated anti-integrin α4 antibody (R1/2), anti-paxillin (H-114, Santa Cruz Biotechnology), anti-talin (H-300, Santa-Cruz) and TOPRO-3 (Invitrogen). Human CD11b+ cells were incubated in non-immune human IgG (10 μg/ml in 5% BSA, 30 min room temperature) and stained with the following: anti-integrin α4 (N-19, Santa Cruz), anti-CXCR4 (12G5, BD Bioscience) and TOPRO-3. Murine cells were incubated in 5% BSA for 30 min at room temperature followed by FITC-conjugated anti-integrin α4 antibody (R1/2), anti-paxillin (H-114, Santa Cruz Biotechnology) and TOPRO-3. Samples were analyzed by Nikon CS1 spectral confocal on a Nikon TE2000E inverted microscopy. Images were captured using EZ-C13.00 imaging software and analyzed using Metamorph software.

### Immunohistochemistry

Mammary fat pads from three month-old PyMT mice bearing spontaneous mouse breast carcinomas, implanted 21 day LLC tumours and Panc02 tumours (mean mass of each=1.5 g) were cryopreserved in O.C.T., cryosectioned and immunostained for CD11b using M1/70 (BD Bioscience), for F4/80+ using BM8 (eBioscience) and for CD31 using MEC13.3 (BD Bioscience). Slides were counterstained with DAPI or TOPRO-3 (Invitrogen). Tissues were analyzed using Metamorph (Version 6.3r5, Molecular Devices). Haematoxylin and eosin staining was performed by the Moores UCSD Cancer Centre Histology Shared Resource. Metastases were quantified by immunostaining with rabbit anti-cow keratin antibody (DAKO). All experiments were performed 3 times unless otherwise indicated. Data were analyzed for statistical significance with an unpaired two-tailed Student's t-test or analysis of variance (ANOVA) coupled with posthoc Tukey's test for multiple pairwise comparisons. P<0.05 was considered to be significant.

### Isolation of Monocytic Cells and Bone Marrow Transplantation

C57Bl6 mice were from Charles River, beta-actin EGFP and Tie2Cre mice were from Jackson Laboratories, integrin α4Y991A mice were derived as described (Feral, et al., 2006), and integrin α4^{loxp/loxp} mice were obtained from Thalia Papayannopoulou, University of Washington, Seattle. Male Tie2Cre mice were crossed with integrin α4^{loxp/loxp} mice. BMDCs were aseptically harvested from 6-8 week-old female mice by flushing leg bones of euthanized mice with phosphate buffered saline (PBS), 0.5% BSA, 2 mM EDTA, incubating in red cell lysis buffer (155 mM NH₄Cl, 10 mM NaHCO₃ and 0.1 mM EDTA) and centrifuging over Histopaque 1083. Approximately 5×10⁷ BMDC were purified by gradient centrifugation from the femurs and tibias of a single mouse. Two million cells were intravenously injected into tail veins of each lethally irradiated (1000 rad) syngeneic recipient mouse. After 4 weeks, tumour growth in C57Bl6 mice transplanted with BM from α4Y991A, WT or α4^{loxp}Cre− and α4^{loxp}Cre+ littermates was compared.

### Gene and Protein Expression

Total RNA was isolated from tissue or cells using ISOGEN (Nippon Gene). cDNA was prepared form 1 μg RNA from each sample and qPCR was performed using primers for SDF-1α, IL-1β, TNF-α, IL-8 and IL-6 from Qiagen (QuantiTect Primer Assay). qPCR for VEGF-A expression was performed using the following primers: sense: GCTGTGCAGGCTGCTCTAAC anti-sense: CGCATGATCTGCATGGTGAT. Transcript levels were normalized to GAPDH expression. Similar studies of gene expression were performed in LLC and Panc02 cells. SDF-1α and IL-1β protein levels were determined in lysates of cultured cells, whole tumours or from tumour derived CD11b+ cells using Quantikine mouse SDF-1α and IL-1β kits (R&D Systems).

### Adhesion Assays

TCM was prepared from serum-free media cultured on LLC cells for 18 h and filtered through 0.22 μm filters. 10⁵ calcein-AM labelled human or murine CD11b+ cells in TCM or DMEM containing 200 ng/ml SDF-1α, IL-1β, IL-6, IL-8 or VEGF-A (R&D Systems) were incubated on HUVEC monolayers or on plastic plates coated with 5 μg/ml recombinant soluble VCAM-1 (R&D Systems). Adherent cells were quantified using a fluorescence activated plate reader (GeniosPro, TECAN). In some assays, cells were also incubated in 25 μg/ml anti-integrin α4β1 (PS2 for murine cells and HP2/1 for human cells, gifts from Biogen-Idec) and isotype control antibodies (IgG2bk for murine cells and IgG1 for human cells). In some studies, cells were incubated in 10 μM pan PI3K inhibitors LY294200 or TG020, in PI3kinase α inhibitor PI3K75, PI3kinase γ/δ inhibitor TG115 and an inert control.

### Immunoprecipitation

BM monocytic cells from WT or α4Y991A mice were isolated as described above and treated with either DMEM or TCM for 30 min at 37° C. Cells were rinsed with cold PBS and lysed in Tris-buffered saline containing 1% CHAPS, 20 mM β-glycerophosphate, 1 mM Na₃VO₄, 5 mM NaF, 100 ng/ml microcystin-LR, and protease inhibitor cocktail. Clarified cell lysates were immunoprecipitated as follows: 1 mg total protein was precleared with 10 μl protein G-conjugated Dynabeads (Invitrogen) for 1 hr with rotation. Cleared lysates were incubated with 5 μg of rat anti-α4β1 (PS/2) antibody overnight, followed by adding 25 μl of protein G-conjugated Dynabeads for 3 h with rotation. Beads were washed three times with 1 ml cold PBS containing protease inhibitor cocktail. Protein precipitates were electrophoresed on 10% SDS-PAGE gels and immunoblotted with anti-integrin α4 (C-20, Santa Cruz Biotechnology), anti-talin (Clone TD77, Chemicon) or anti-paxillin (H-114, Santa Cruz Biotechnology) antibodies. Immune complexes were visualized using an enhanced chemiluminescence detection kit (Pierce).

### In Vivo Myeloid Cell Trafficking Studies

CD11b+ cells from C57B16 mice were fluorescently-labelled with green carboxy-fluorescein diacetate, succinimidyl ester (CFDA SE, 5 μM, Invitrogen), and CD11b+ cells from α4Y991A mice were labelled with cell tracker red (5 μM CMTPX™, Invitrogen). Cell viability was tested with Trypan blue staining and in adhesion assays in vitro. Labelled cells were mixed 1:1 and 4×10⁶ cells were injected intravenously into the tail vein of mice bearing LLC carcinomas implanted under dorsal skin-fold window chambers. Accumulated fluorescent cells were quantified after one hour. Alternatively, 10⁷ CD11b+ cells C57Bl6 or α4Y991A mice were labelled with CFDA and were injected intravenously into mice bearing subcutaneous LLC tumours. Fluorescent cells accumulating in tumours and spleens were quantified 24 h later by excising tissues, preparing single cell suspensions and performing FACs analysis at 488 nm.

### Migration Assays

Both sides of Costar Transwell inserts (8 μm pore) were coated with 5 μg/ml VCAM-1 or vitronectin. Lower chambers were filled with TCM or DMEM. One hundred thousand purified CD11b+ cells were added to the top chambers of transwells and incubated in a humidified atmosphere at 37° C. for 4 h. Migration of CD11b+ cells was measured by counting cells on the underside of the transwell filter.

### In Vivo Angiogenesis Assays

Growth Factor-depleted Matrigel (BD Bioscience) containing 400 ng SDF-1α, IL-1β (R&D Systems) or saline in 400 μl was injected subcutaneously into C57Bl6 mice (n=6) transplanted with BM from beta-Actin EGFP+ mice. One week later, Matrigel plugs were excised, cryopreserved, sectioned and immunostained for the presence of myeloid cells and blood vessels. In additional studies, 1×10⁶ BM derived CD11b+ cells from WT or α4Y991A mice were mixed with 400 μl Matrigel and implanted in C57Bl6 mice. Cryosections from Matrigel plugs excised after 10 days in vivo were immunostained to detect CD31+ blood vessels and were counterstained with DAPI (blue) (n=6).

Purification of CD11b+ Cells

CD11b+ cells from human buffy coats or murine BM were purified by anti-CD11b magnetic bead affinity chromatography (Miltenyi Biotec). To assess the purity of the CD11b+ cell population, allophycocyanin (APC) labelled anti-CD11b antibodies were added together with the magnetic beads and flow cytometry was performed.

### Example 2

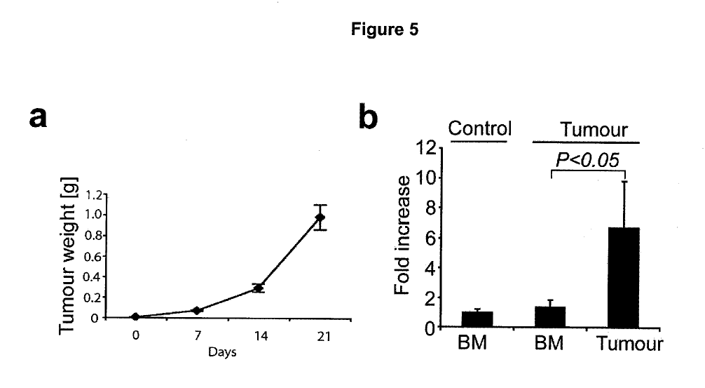

To explore mechanisms regulating myeloid cell trafficking to tumours, we first quantified myeloid cells in murine tumours. Similar to human tumours¹²⁻¹⁵, ten to fifteen-fold more CD11b+ cells accumulated in spontaneous murine breast, orthotopic lung, orthotopic pancreatic and subcutaneous lung carcinomas than in corresponding normal tissues (FIG. 1a). CD11b+ cells were rapidly recruited to tumours, increasing from 1% of the cell population in normal tissues to greater than 62% in Lewis lung carcinoma (LLC) cells (FIG. 1b). Myeloid cell influx preceded tumour angiogenesis and growth (FIG. 1c, FIG. 5), supporting the concept that tumour inflammation precedes and promotes the angiogenic switch²⁻³.

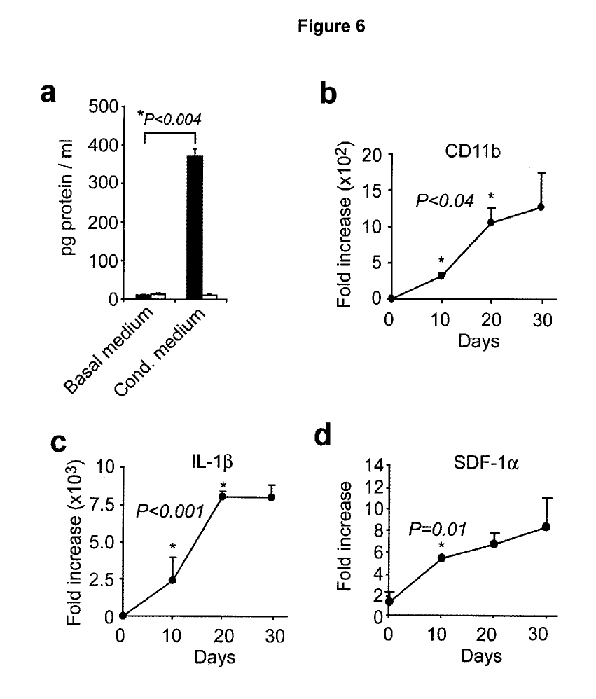

To determine whether specific inflammatory mediators recruit myeloid cells to tumours, we quantified the expression of such factors in LLC and Panc02 pancreatic tumour cells in vitro and tumours in vivo by qPCR and ELISA. Tumour cells in vitro and tumours in vivo expressed SDF-1α, VEGF-A and TNFα, while normal tissues did not. Only tumours in vivo expressed IL-1β and IL-6 (FIG. 1d, FIG. 6). Surprisingly, the only source of IL-1β and IL-6 in tumours was CD11b+ cells, while the exclusive source of SDF-1α in vivo was CD11b− tumour (and stromal) cells (FIG. 1e, FIG. 6). Protein expression levels of these factors were similar to mRNA levels (FIG. 1f-g, FIG. 6). Thus, tumour cells express a variety of structurally diverse pro-inflammatory factors including VEGF-A, SDF-1α and TNFα, while tumour-recruited myeloid cells express a distinct set of inflammatory factors such as IL-1β and IL-6.

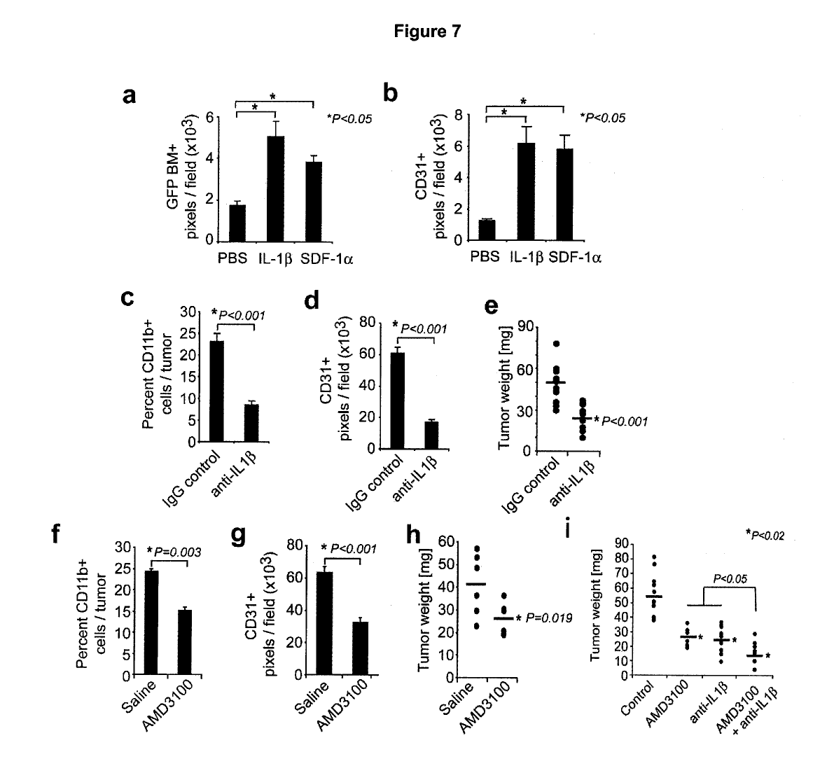

We found that both tumour and myeloid cell-derived inflammatory factors directly recruit myeloid cells, thereby promoting angiogenesis and tumour growth in vivo. Purified IL-1β or SDF-1α stimulated myeloid cell invasion and angiogenesis in vivo (FIG. 7). These factors also promote CD11b+ cell recruitment to LLC tumours, angiogenesis and tumour growth, as antagonists of IL-1β or SDF-1α blocked these processes, either alone or in combination (FIG. 7). As a combination of SDF-1α and IL-1β antagonists substantially suppressed tumour growth (FIG. 7), these results indicate that blockade of both initial tumour cell-mediated and secondary myeloid cell-mediated inflammation may provide significant anti-tumour therapeutic benefit.

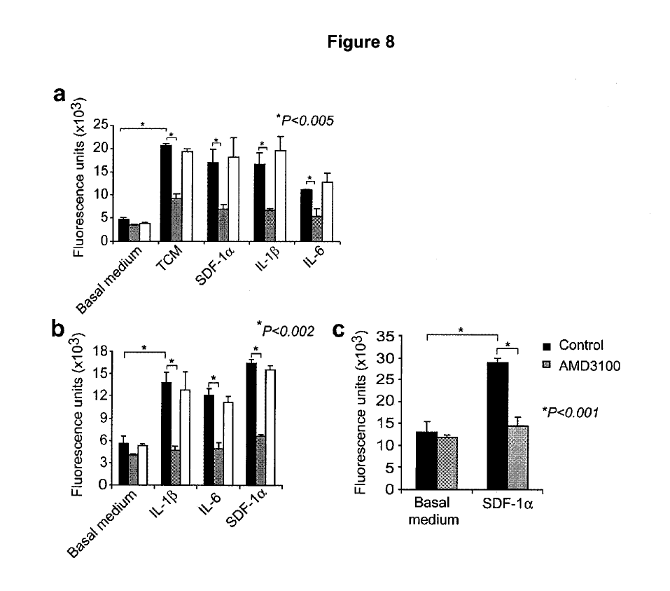

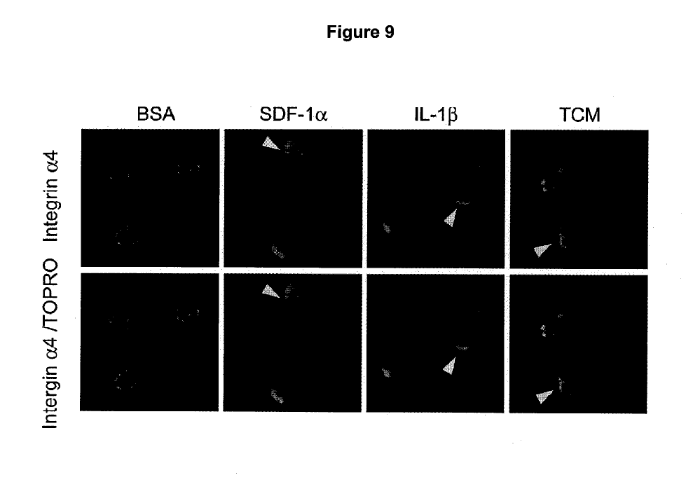

Immune cell extravasation from the circulation can depend on activation of α4 or β2 integrins by chemokines, with subsequent adhesion to vascular endothelium and extravasation¹⁶⁻¹⁸. We examined whether tumour-derived inflammatory factors promote integrin-dependent myeloid cell adhesion to endothelium. Fluorescently labelled, human and murine CD11b+ cells adhered to vascular endothelium and to recombinant VCAM, an α4 ligand expressed by endothelium in tumours and inflamed tissues, after stimulation by tumour-conditioned medium (TCM), purified SDF-1α, IL-1β, IL-6, IL-8 or VEGF-A (FIG. 2a, FIG. 8). This adhesion event was strictly mediated by integrin α4 rather than other myeloid cell integrins, as antibody antagonists of α4 but not αMβ2,¹⁹ inhibited adhesion of human and murine CD11b+ cells to vascular endothelium regardless of the inflammatory stimulus (FIG. 2a, FIG. 8). Thus, structurally diverse inflammatory factors such as SDF-1α, VEGF, IL-1β, IL-8 and IL-6, which activate unique signalling pathways mediated by G-protein coupled receptors (GPCR), Type III tyrosine kinase receptors, Toll-like receptors (TLR) and type I cytokine receptors, uniformly stimulate integrin α4 activity and promote integrin α4-dependent adhesion of myeloid cells to endothelium.

We next asked which receptor-mediated effectors promote integrin α4-dependent cell adhesion. Myeloid cells from MyD88−/− mice, which are defective in TLR/IL-1β receptor signalling²⁰⁻²¹, failed to adhere to endothelium in the presence of IL-1β but adhered normally in the presence of SDF-1α and TCM (FIG. 2b), indicating that IL-1R-mediated signal transduction is required for IL-1β-induced adhesion. Similarly, pertussis toxin (FIG. 2b) and AMD3100²² (not shown) inhibited SDF-1α, and TCM-induced but not IL-1β induced adhesion, indicating that CXCR4-mediated signal transduction is necessary for SDF-1α induced cell adhesion to endothelium. Interestingly, we found that these diverse inflammatory cytokines each stimulated PI3kinase γ-dependent integrin α4 adhesion. PI3kinase γ, but not other isoforms of PI3kinase, was found to promote integrin-mediated adhesion downstream of SDF-1α, IL-1β, IL-6, IL-8 and VEGF, since PI3kinase γ null myeloid cells failed to adhere to endothelium, regardless of the stimulus (FIG. 2c). Furthermore, inhibitors of the γ/δ isoforms of PI3kinase, but not of the α/β isoforms of PI3kinase,²³⁻²⁴ suppressed wildtype myeloid cell adhesion (FIG. 2c). Therefore, diverse inflammatory receptors activating unique signalling pathways each converge on PI3kinase γ-, integrin α4-mediated myeloid cell attachment to endothelium.

Chemokine signalling can promote conformational changes in integrin β chains that rapidly unfold integrin heterodimers and increase affinity for ligand²⁵. These changes can be detected by binding of a monoclonal antibody, HUTS21, to newly revealed epitopes on the human β1 integrin subunit²⁶. To determine whether tumour derived factors induce integrin conformational changes, we stimulated human CD11b+ cells with SDF-1α, IL-1β, IL-8, IL-6 or Mn2+ (a positive control²⁶) and performed flow cytometry to detect binding of HUTS21 and P4C10, an antibody that recognizes 131 integrin regardless of conformation. Each inflammatory factor stimulated HUTS21 but had no effect on P4C10 binding, indicating that these factors activate myeloid cell β1 integrins without affecting integrin expression (FIG. 2d).

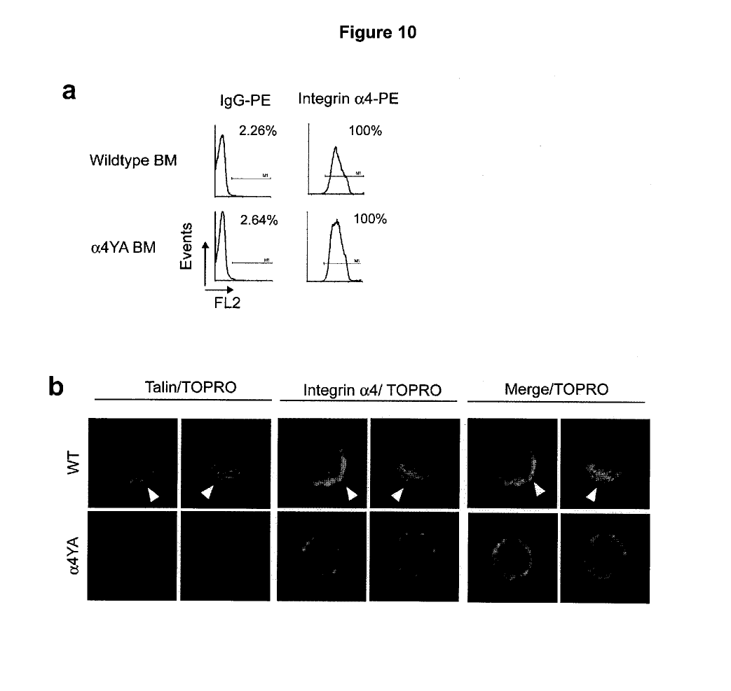

Integrin activation rapidly results in upregulation of integrin avidity and clustering within the plane of the lipid bilayer²⁵. We found that inflammatory factors stimulated integrin α4 clustering in murine and human CD11b+ cells (FIG. 2e, FIG. 10). Upon stimulation with immobilized SDF-1α or IL-1β, integrin α4β1 was clustered in the plane of the membrane; upon SDF-1α stimulation, integrin α4 co-clustered with the SDF-1α receptor CXCR4 (FIG. 2f), indicating that CXCR4 and integrin α4 interact closely following chemokine receptor ligation. Receptor-mediated signalling is required for SDF-1α-induced integrin clustering, as pertussis toxin blocked SDF-1α-induced integrin clustering (FIG. 2g). Additionally, IL-1β-induced integrin clustering was inhibited in MyD88−/− cells (FIG. 2g). PI3kinase γ isoform inhibitors also blocked integrin clustering induced by either cytokine (FIG. 2g), indicating that myeloid cell integrin activation and clustering are regulated by diverse receptor signalling pathways that all depend on PI3kinase γ activation.

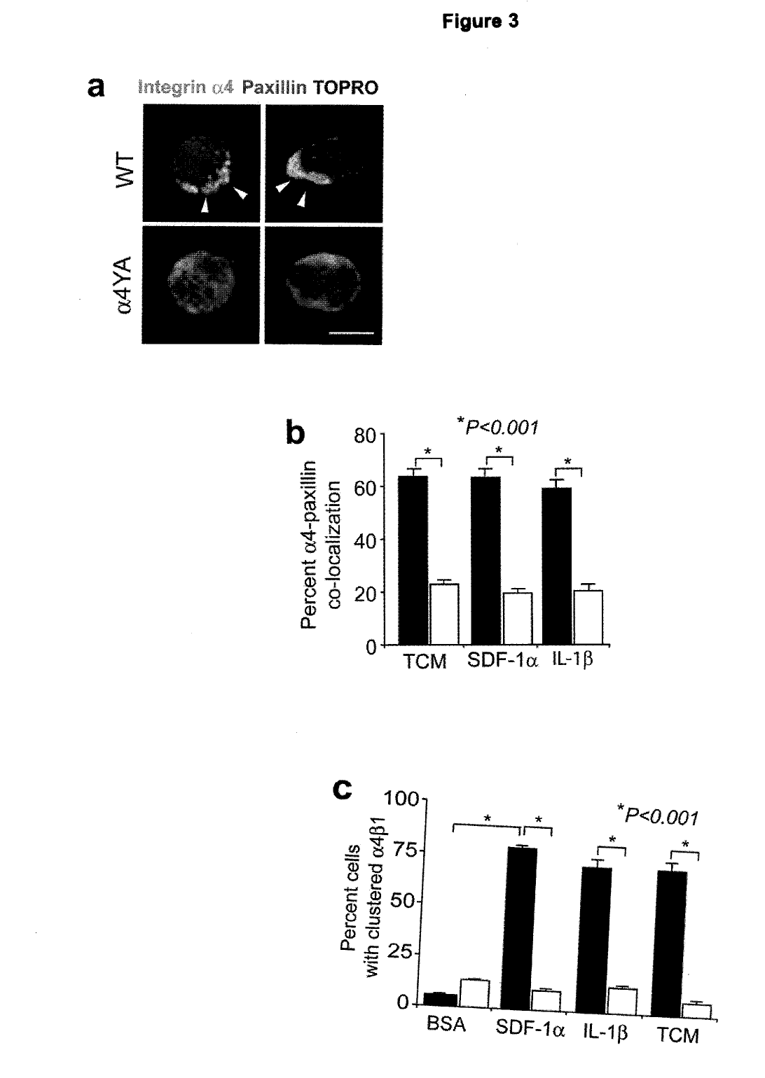

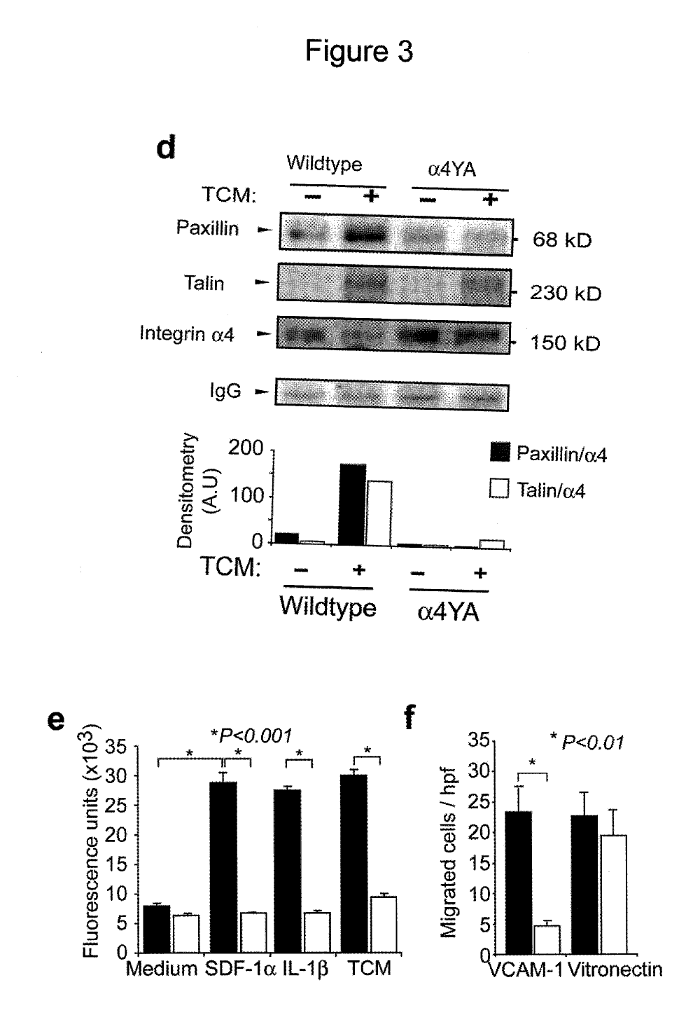

Activation of integrin α4β1-mediated adhesion depends on association of talin with an NPXY domain in β1 cytoplasmic tails and biological activity depends on association of paxillin with tyrosine 991 in the integrin cytoplasmic tail²⁷⁻²⁹. In mice with an integrin α4Y991A cytoplasmic tail knockin mutation (α4Y991A), paxillin fails to bind to the integrin α4 cytoplasmic tail of immune cells, thereby inhibiting immune cell extravasation in response to thioglycollate-induced intraperitoneal inflammation³⁰. Although inflammatory mediators stimulate integrin clustering in wildtype cells, they fail to promote integrin clustering and co-clustering with paxillin or talin in cells from α4Y991A mice (FIG. 3a-c, FIG. 10), even though integrin α4 expression levels are identical in wildtype and α4Y991A cells (FIG. 10). Furthermore, paxillin and talin can be co-immunoprecipitated with integrin α4β1 from stimulated wildtype, but not α4Y991A CD11b+ cells (FIG. 3d). Additionally, α4Y991A CD11b+ cells fail to adhere to endothelium in response to inflammatory factors (FIG. 3e) and exhibit defective cell migration (FIG. 3f). These results demonstrate that paxillin-integrin α4 interactions are required for integrin α4-mediated myeloid cell adhesion to endothelium.

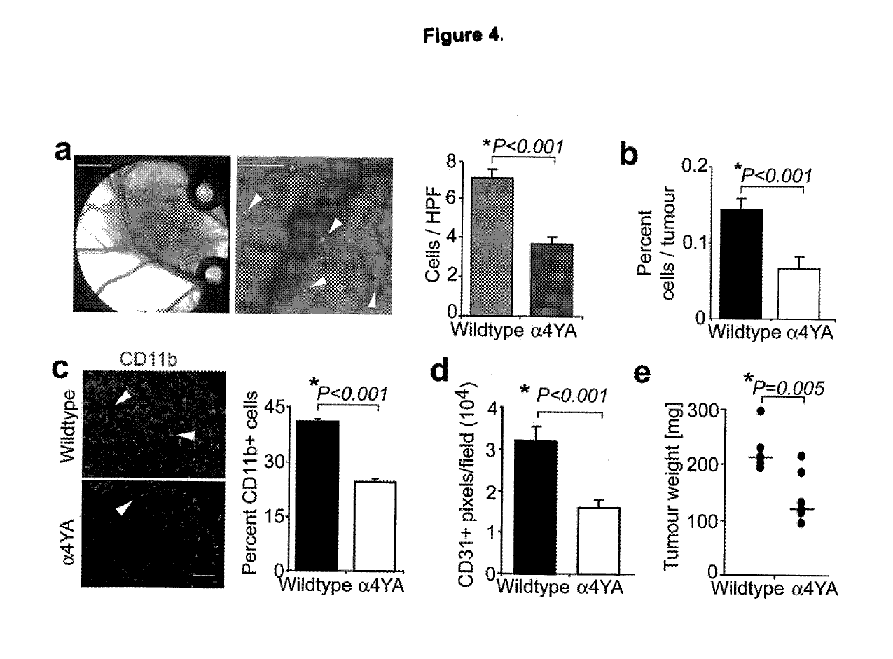

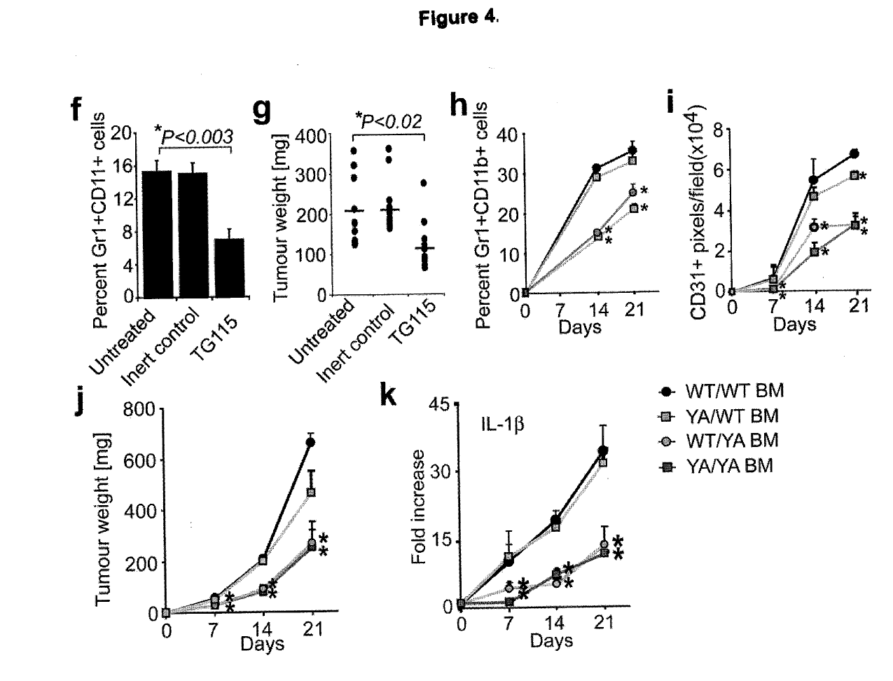

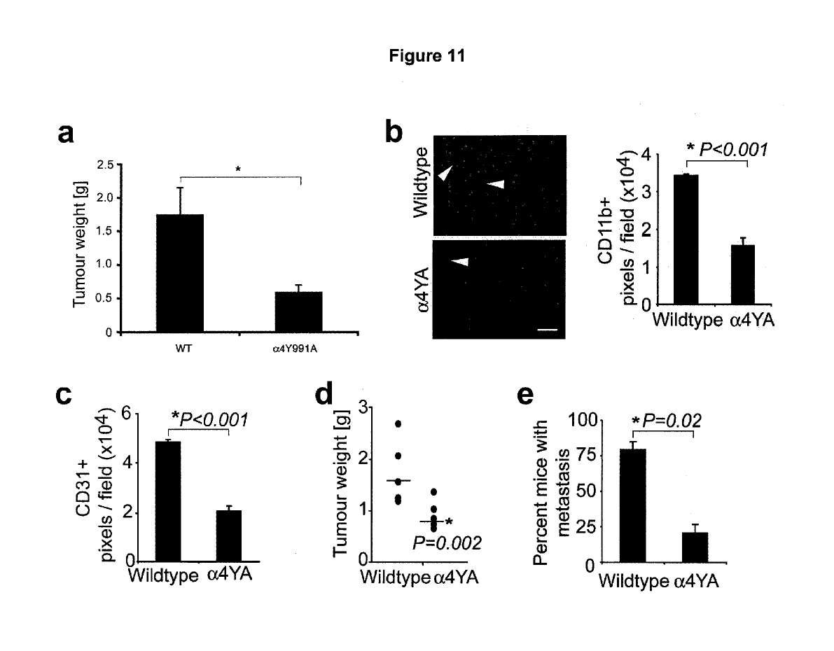

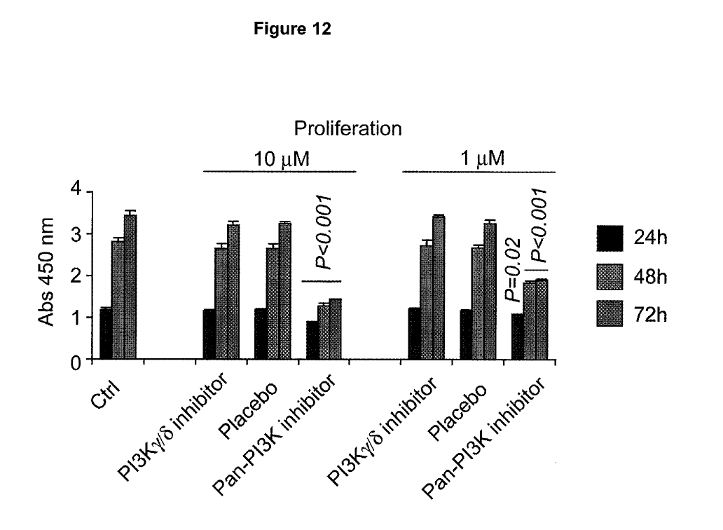

To assess the requirement for myeloid cell integrin α4 activation in myeloid cell recruitment to tumours in vivo, we adoptively transferred α4Y991A and wildtype CD11b+ cells into tumour-bearing wildtype animals. α4Y991A cells failed to infiltrate tumours, providing strong evidence for a role of integrin α4 activation in extravasation and tumour infiltration (FIG. 4a-b). In fact, CD11b+ cell infiltration of LLC and Panc02 tumours (FIG. 4c, FIG. 11), tumour neovascularization (FIG. 4d, FIG. 11) and tumour growth (FIG. 4e, FIG. 11) were all strongly suppressed in α4Y991A versus wildtype animals. Importantly, significantly fewer metastases developed in α4Y991A than in wildtype animals (FIG. 11). We also found that PI3kinase γ inhibitors suppressed CD11b+ cell infiltration of tumours and tumour growth (FIG. 4f-g) yet had no direct effect on tumour cell proliferation (FIG. 12). Together, these studies indicate that activation of myeloid cell integrin α4-mediated tumour infiltration via PI3kinase γ is critical for tumour inflammation and progression of tumours in vivo.

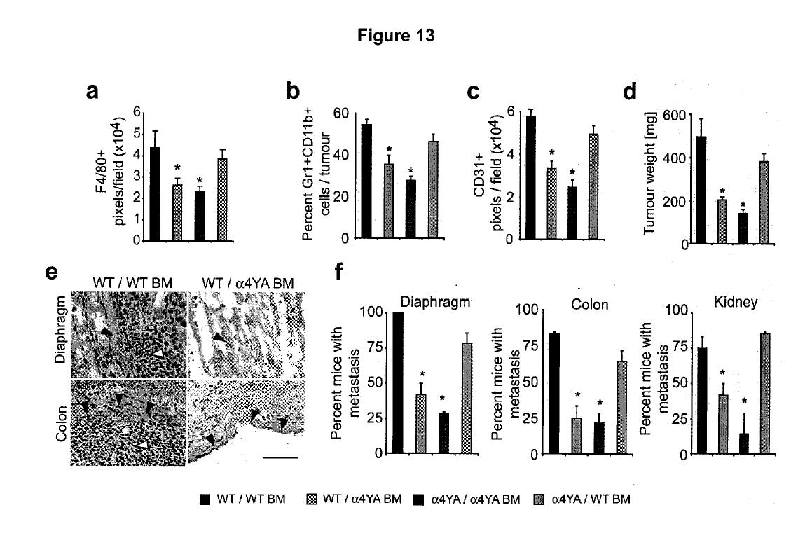

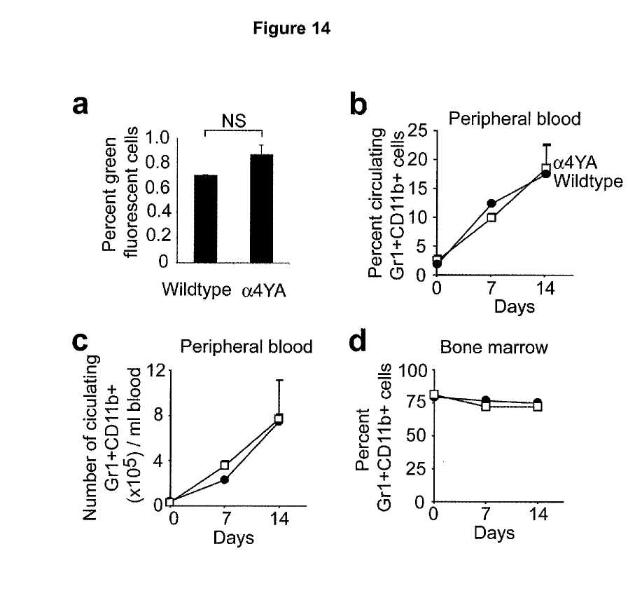

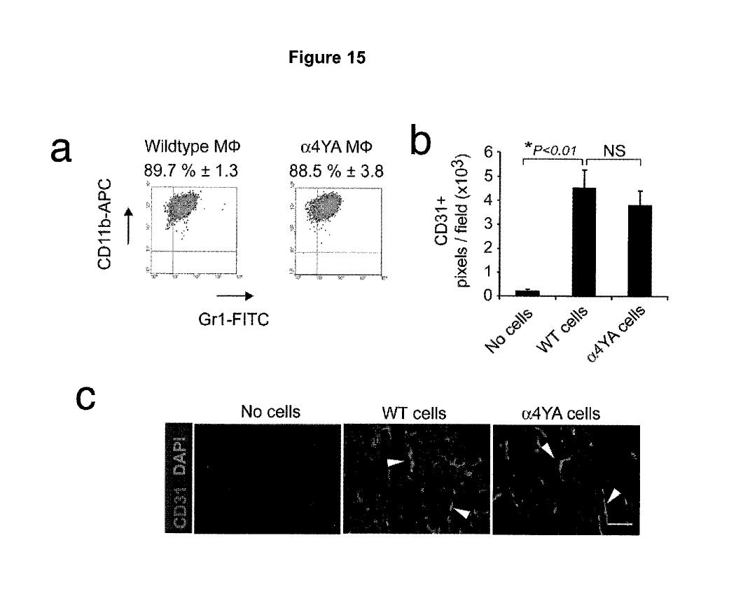

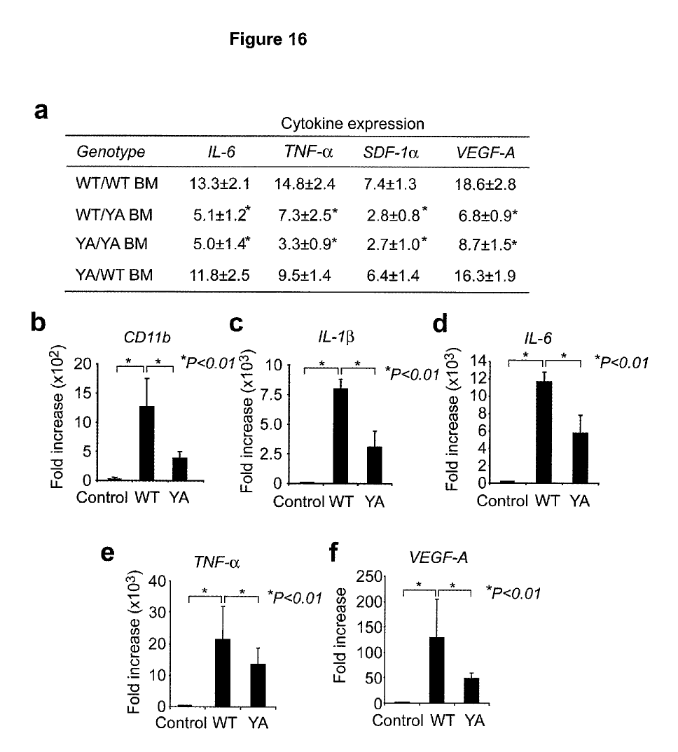

LLC and Panc02 tumour suppression in α4Y991A mice results from a defect in myeloid cell trafficking, rather than any significant defect in the stromal cell compartment of these animals, as recruitment of Gr1+CD11b+ cells and F4/80+ macrophages to tumours (FIG. 4h, FIG. 13), tumour angiogenesis (FIG. 4i) and tumour growth (FIG. 4j, FIG. 13) and metastasis (FIG. 13) were suppressed when tumours were grown in wildtype animals transplanted with α4Y991A bone marrow (BM). Reduced tumour myeloid cell content and angiogenesis can be attributed to reduced myeloid cell recruitment, as there are no differences in the numbers of circulating or BM resident Gr1+CD11b+ cells in normal or tumour bearing wildtype and α4Y991A mice (FIG. 14), and α4Y991A and wildtype myeloid cells are equally able to differentiate into macrophages in vitro or to stimulate angiogenesis in vivo (FIG. 15). A consequence of reduced myeloid cell recruitment to tumours is decreased expression of inflammatory factors IL-1β and IL-6 in the tumour microenvironment (FIG. 4k, FIG. 16). SDF-1α, TNFα, and VEGFA levels were also reduced in tumours from α4Y991A BM transplanted mice in proportion to the reduction in tumour growth rate (FIG. 16).

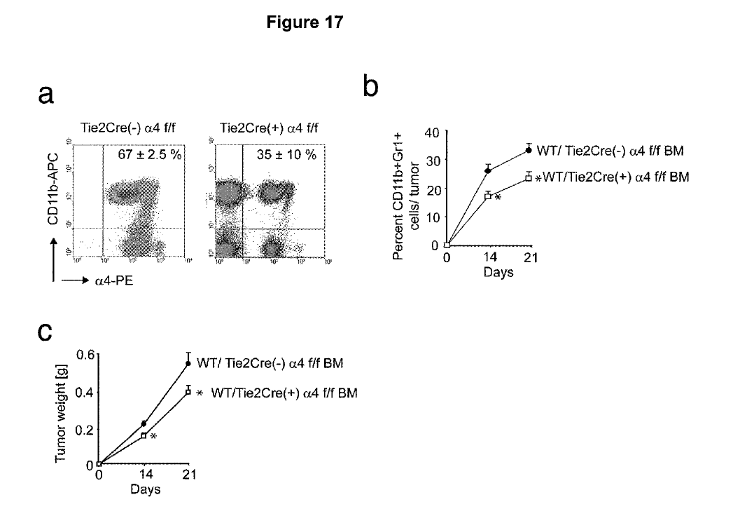

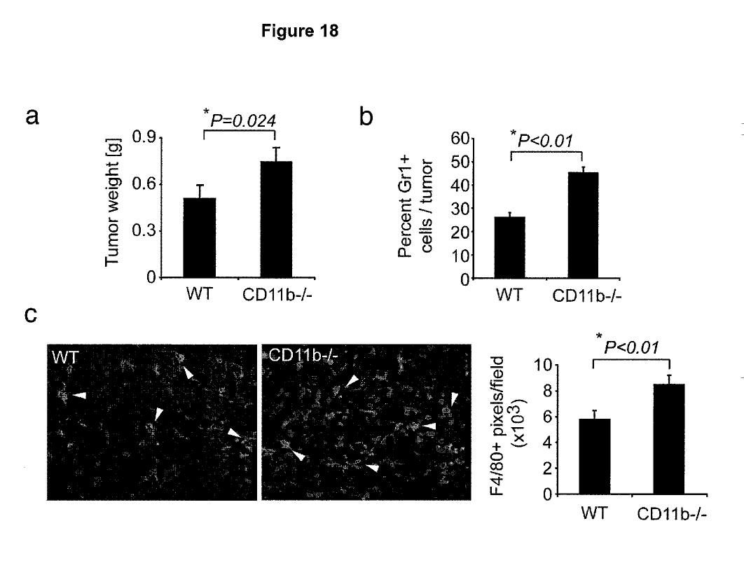

Decreased expression of integrin α4 on myeloid cells also suppresses tumour inflammation, as tumours grown in wildtype animals transplanted with bone marrow from TieCre+α4^{loxp/loxp} exhibited reduced myeloid cell recruitment and growth. CD11b+ cells from TieCre+α4^{loxp/loxp} animals express significantly reduced integrin α4β1 (54%+) than wildtype cells (100%+) (FIG. 17). In contrast, decreased expression of integrin αMβ2 promotes rather than inhibits tumour inflammation, as CD11b−/− mice lacking αMβ2 integrin exhibit enhanced, rather than suppressed, myeloid cell recruitment, angiogenesis and tumour growth (FIG. 18).

Together, our studies indicate that myeloid cell integrin α4β1 activation by a central PI3kinase γ dependent-signalling pathway plays a critical role in tumour inflammation and growth. This takes place even though tumour cells initiate recruitment of pro-angiogenic myeloid cells by expressing factors that include SDF-1α, TNFα and VEGF-A, and infiltrating myeloid cells exacerbate recruitment by expressing IL-1β, IL-6 and other factors. Together, these studies indicate that therapeutic agents directed at inhibiting PI3Kinase γ or integrin α4 could provide substantial benefit in reducing tumour inflammation, growth and metastasis.

### Example 3

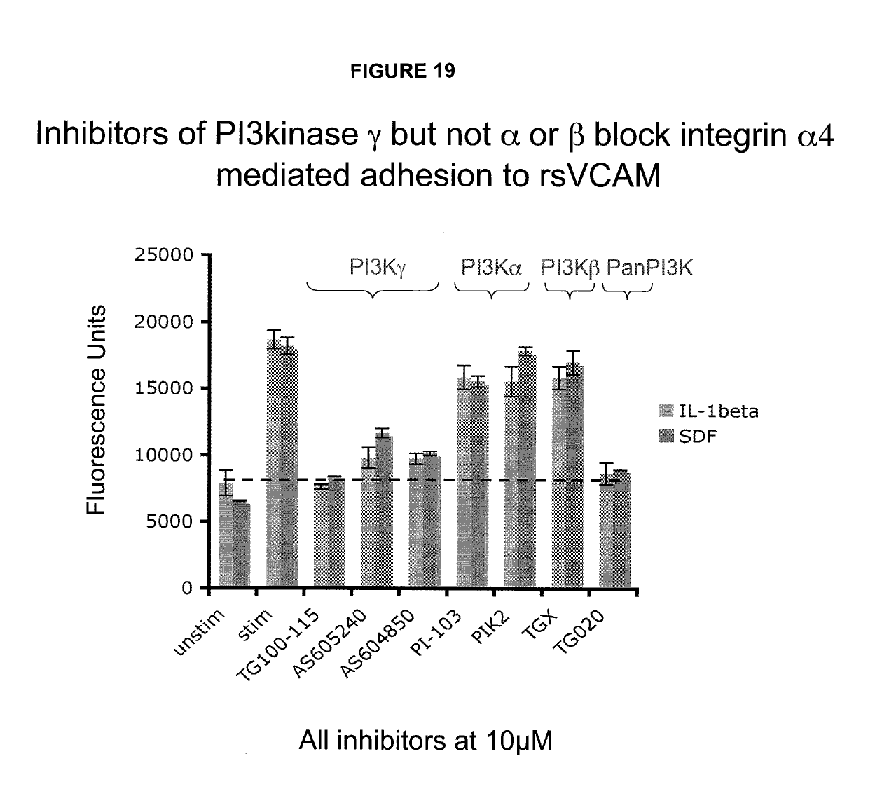

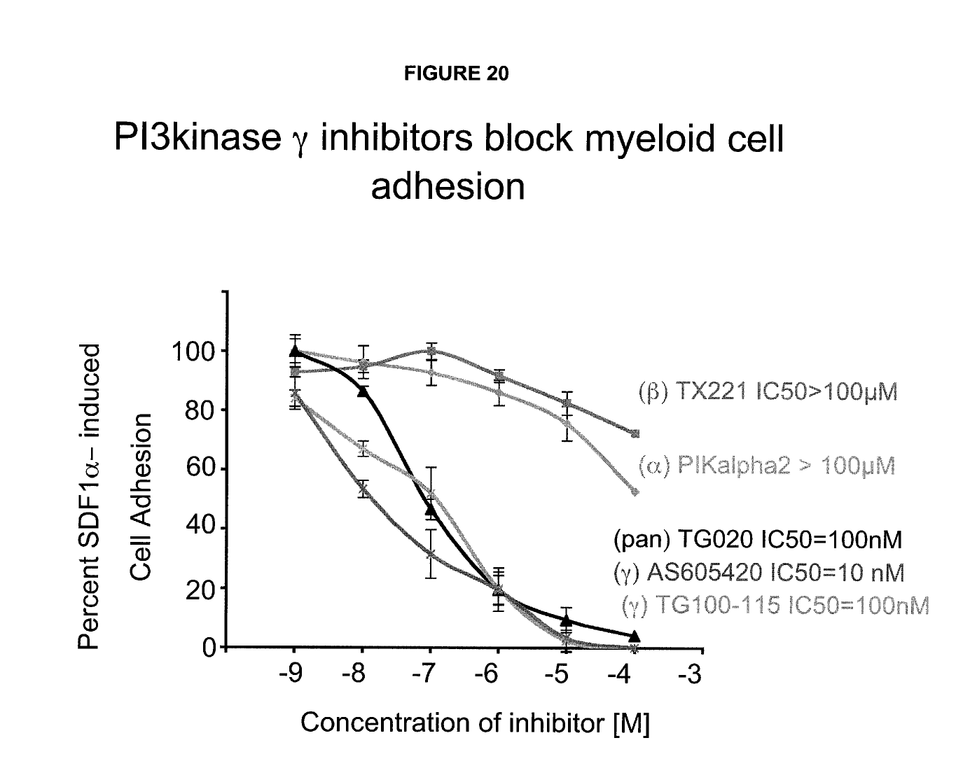

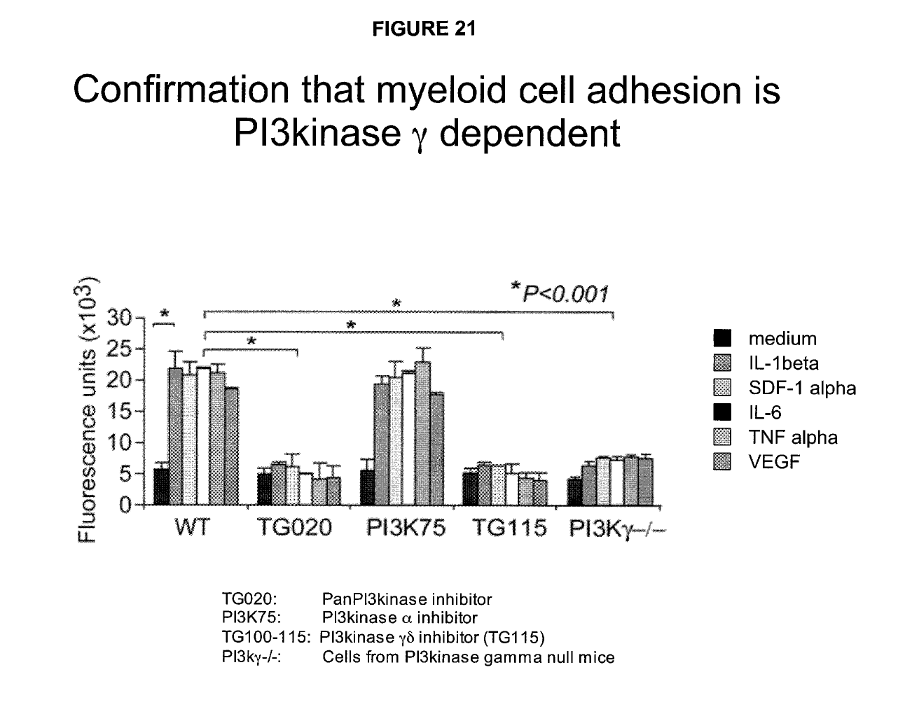

PI3kinase isoform selective inhibitors were evaluated for their effects on integrin α4b1 mediated myeloid cell adhesion to endothelium and to purified recombinant soluble VCAM. Data are shown in FIGS. 19-21.

Myeloid cells were purified from bone marrow as previously described and were labeled with calcein-AM. Labelled cells were placed in medium containing no additives, SDF1, IL-1 beta, IL-8, IL-6, VEGF or TNFa containing PI3kinase inhibitors at various concentrations and were allowed to adhere to 48 well plastic plates coated with recombinant soluble VCAM or monolayers of human umbilical vein endothelial cells for 30 minutes. Cells were well washed, then quantified in a fluorimeter. Inhibitors are: TG020, a pan-PI3kinase inhibitor and TG100-115, a PI3kinase gamma/delta inhibitor from Targegen, Inc.; AS605420 and AS604850, PI3kinase gamma inhibitors first described by Serono and purchased from Echelon, Inc.; PI-103 and PIK3alpha2, PI3kinase alpha selective inhibitors purchased from Echelon; PIK75, a PI3kinase alpha selective inhibitor obtained from Targegen; TGX115, a PI3kinase beta selective inhibitor purchased from Echelon. PI3kinase gamma−/− mice were obtained under MTA from Dr. Josef Penninger at the Molecular Biology Institute of Vienna, Austria.

### Example 4

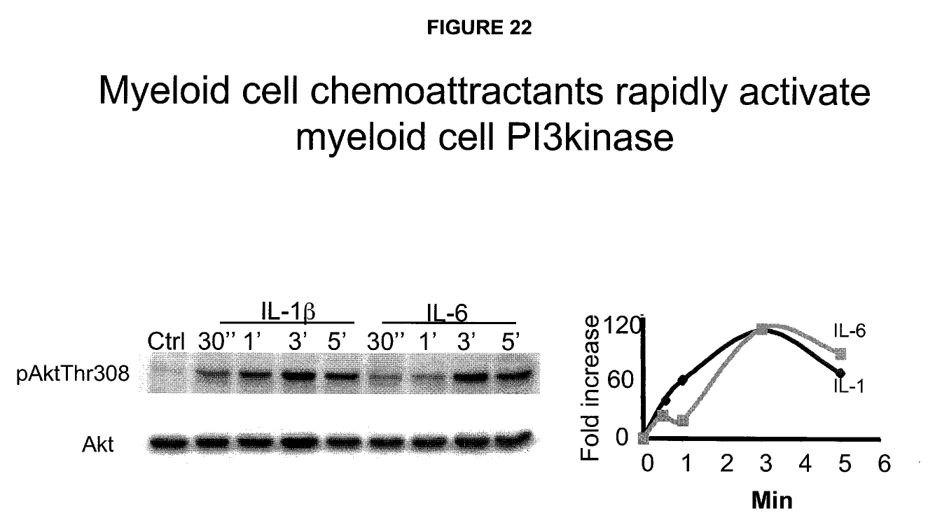

Myeloid cell chemoattractants rapidly activate myeloid cell PI3kinase (FIG. 22). WT or PI3kinase gamma−/− myeloid cells were treated for 30 sec, 1,3, or 5 minutes with chemoattractants including IL-1 beta and IL-6 and solubilized in RIPA buffer. Equal protein amounts were loaded on gels, electrophoresed and transferred to nitrocellulose. Blots were incubated and in anti-phospho-Akt, anti-Akt.

### Example 5

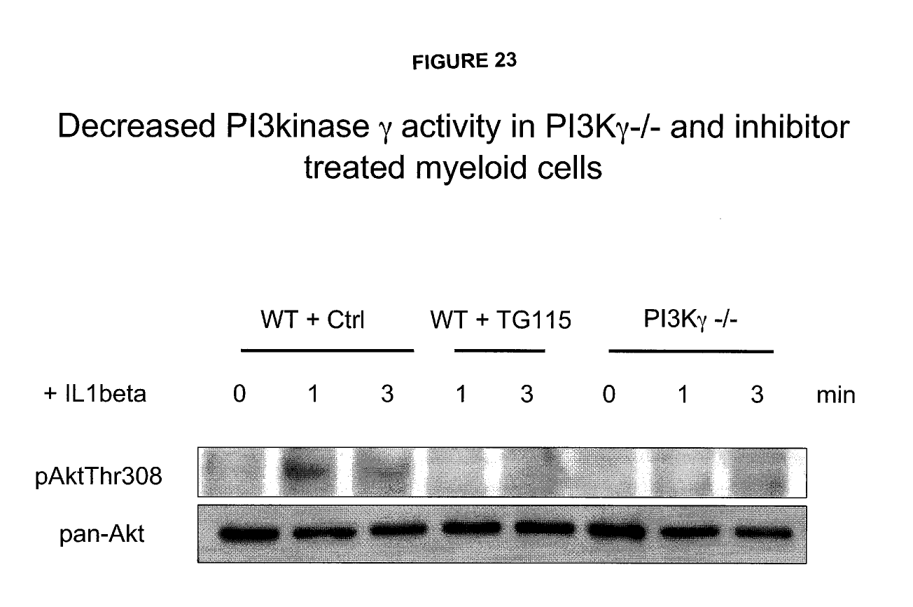

Decreased PI3kinase gamma activity in PI3 Kgamma −/− and inhibitor treated myeloid cells (FIG. 23). WT or PI3kinase gamma−/− myeloid cells were treated for 0-3 minutes with IL-1 beta and PI3kinase gamma selective inhibitors or inert controls, then solubilized in RIPA buffer. Equal protein amounts were loaded on gels, electrophoresed and transferred to nitrocellulose. Blots were incubated and in anti-phospho-Akt, anti-Akt.

### Example 6

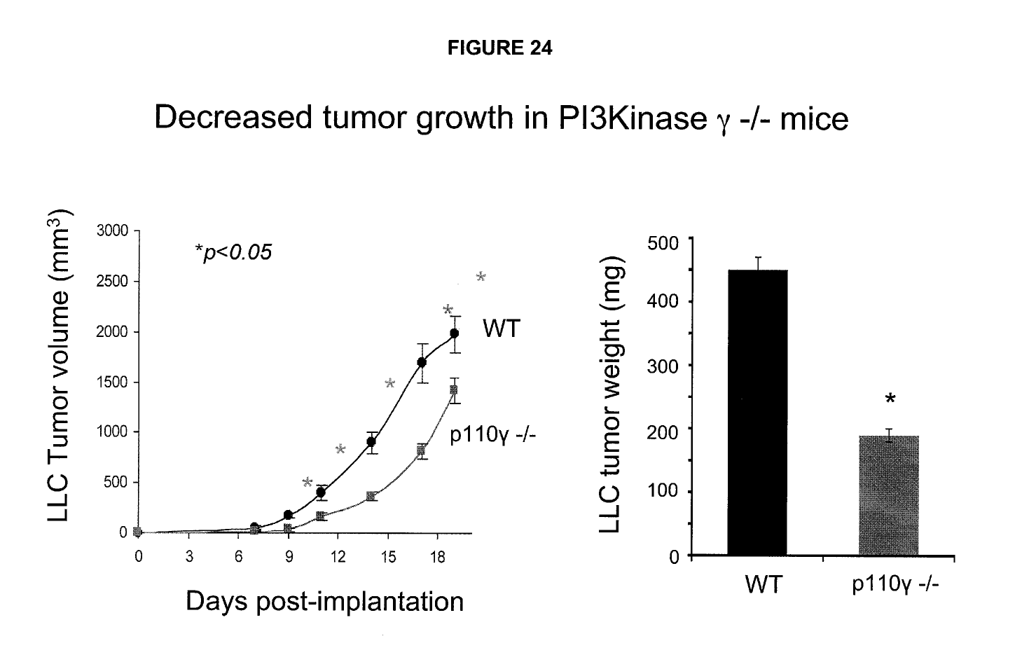

Evaluation of tumor growth in PI3kinase gamma−/− mice (FIG. 24). 5×10E5 Lewis lung carcinoma tumor cells (C57 BL6 background) were injected subcutaneously into C57Bl6 strain wildtype and PI3kinase gamma−/− mice (n=7). Tumor volume was determined using calipers every other day for up to three weeks. Animals were sacrificed and tumor weights determined.

### Example 7

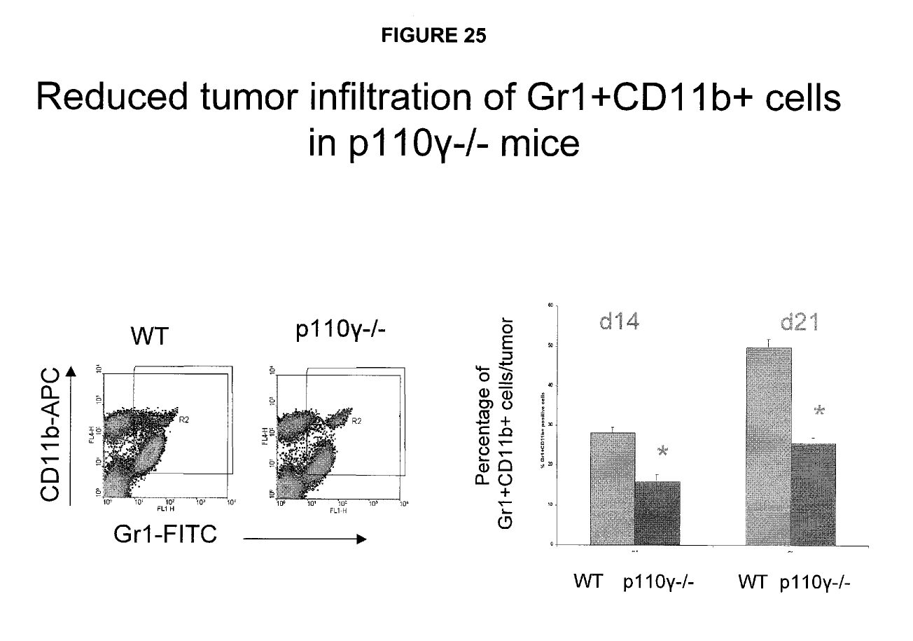

Quantification of tumor infiltrating myeloid cells (FIG. 25). Tumors removed at 14 days were dissociated into single cell suspensions by collagenase digestions and CD11b+ and Gr1+ cells in tumors were quantified by FACs analysis. Total CD11b+GR1+ cells were significantly reduced in p110gamma −/− animals at day 14 and d21 of tumor growth. The greatest reduction in tumor myeloid cells appears to be in the CD11b+Gr1med population, which we have found is the F4/80+ macrophage population.

### Example 8

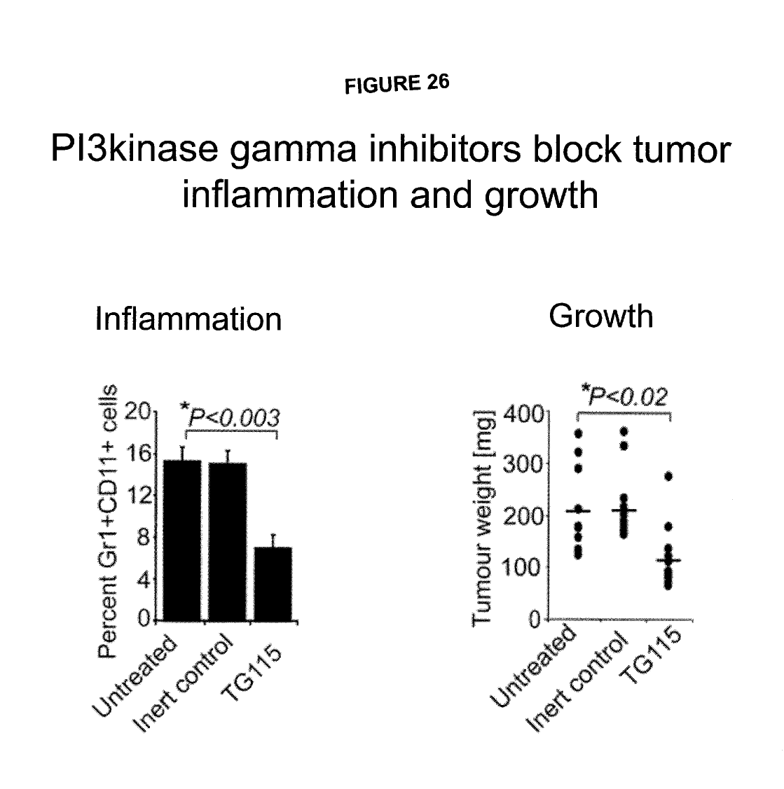

Reduced LLC tumor growth in PI3kinase gamma −/− treated mice (FIG. 26). 5×10E5 Lewis lung carcinoma tumor cells (C57 BL6 background) were injected subcutaneously into C57Bl6 strain mice. One group of mice was treated with saline (n=10), one group was treated with 5 mg/kg twice per day by IP injection with an inert control inhibitor (n=10) and one group was treated with 5 mg/kg twice per day by IP injection of the PI3kinase gamma/delta selective inhibitor, TG100-115 (n=10) for two weeks. Tumor weight and myeloid cell infiltrate was assessed.

### Example 9

### Methods Used in Example 10

### Transgenic and Other Animals

Male PyMT+ mice on an FVB background were randomly bred with FVB females lacking the PyMT transgene to obtain female mice heterozygous for the PyMT transgene. Female mice heterozygous for the PyMT transgene were compared to wild type FVB female mice. All PyMT+ females exhibit hyperplasias by 6 weeks of age and the majority exhibit adenomas/early carcinomas by 9 weeks of age and lymph node and lung metastases by 12-15 weeks of age³¹. Integrin α4Y991A mice in the C57BL/6 background were derived as previously described¹⁶. Integrin α4Y991A mice were backcrossed for 8 generations into the FVB lineage and then crossed with FVB PyMT males to achieve PyMT+α4Y991A/α4Y991 female mice for breast tumor development studies. Additionally, male Tie2Cre+ mice were crossed with female integrin α4^{loxp/loxp} mice³ to generate Tie2Cre+ α4^{loxp/+} mice, which were then crossed to with α4^{loxp/loxp} mice to obtain sibling Tie2Cre− α4^{loxp/loxp} and Tie2Cre+ α4^{loxp/loxp} mice for studies. PI3-kinase γ−/− (p110γ−/−)²¹ mice were obtained from Dr. Joseph Penninger of the Institute of Molecular Biotechnology, Vienna, Austria. C57BL/6 mice were obtained from Charles River, and Tie2Cre mice and CD11b−/− mice were from Jackson Laboratories.

### Tumour Studies

C57BL/6 LLC cells and B16 cells were obtained from the American Type Culture Collection (ATCC) and C57BL/6 Panc02 pancreatic ductal carcinoma cells were obtained from the NCI DCTDC Tumour Repository. All cells were cultured in antibiotic- and fungicide-free DMEM media containing 10% serum and tested negative for mycoplasma using the Mycoplasma Plus PCR primer set from Stratagene (La Jolla, Calif.).

Orthotopic pancreatic tumours were initiated by implanting 1×10⁶ Panc02 pancreatic carcinoma cells into the pancreas of syngeneic mice. The abdominal cavities of immunocompetent C57BL/6 mice and integrin α4Y991A mice were opened and the tails of the pancreata were exteriorized. One million Panc02 cells were injected into the pancreatic tail, the pancreas was placed back into the abdominal cavity, and the incision was closed. Pancreata were excised and cryopreserved after 30 days. Lymph nodes and other organs were visible metastases were also cryopreserved. Tumour weight, angiogenesis and CD11b/F4/80 content were quantified as described. Studies were performed twice with n=6.

Subcutaneous tumours were initiated as follows: 5×10⁵ LLC cells or B16 cells were injected subcutaneously into syngeneic (C57BL/6) 6- to 8-week old wildtype (WT), integrin α4Y991A, or PI3-kinase γ−/− (p110−/−) mice. Tumours dimensions were recorded and tumours were excised at 7, 14 or 21 days. Tumour weights were obtained at each time point. Tumours were cryopreserved in O.C.T., solublized for RNA purification or collagenase-digested for flow cytometric analysis of CD11b and Gr1 expression as detailed below. Angiogenesis was measured by CD31 immunostaining. For orthotopic LLC tumors, 5×10⁵ LLC cells were injected in the tail vein and lungs were harvested after 12 days. LLC tumour studies in WT versus α4Y991A animals were performed three times (n=6-8). B16 studies in WT versus α4Y991A animals were performed once (n=8).

### Clinical Specimens

Patients at the Moores UCSD Cancer Center in La Jolla, Calif., underwent planned procedures for breast surgical treatment. All surgeries were performed at the University of California, San Diego and standard techniques were used for resection of breast tissue. Normal tissue was also obtained from patients undergoing breast reduction or prophylactic mastectomy. Specimens were removed, sent to the UCSD Medical Center pathology laboratory for analysis, and reviewed by a pathologist to assess the surgical margin tissue. A sample of tissue was placed on dry ice and stored at −80° C. Tissues not needed for diagnosis were embedded in O.C.T. for cryosectioning. 10 invasive ductal carcinomas and 10 normal tissues were evaluated for the presence of CD11b+ cells by immunostaining of frozen sections.

### Quantification of Myeloid Cells and Blood Vessels in Tissues by Immunohistochemistry

Mammary fat pads from three month-old PyMT mice bearing spontaneous mouse breast carcinomas, LLC tumours grown orthopically in lung for 12 days or subcutaneously in skin for 21 days (with an average mass of 1.5 g), and orthotopic Panc02 tumours grown in the pancreas for 30 days (with an average mass of 1.5 g) were cryopreserved in O.C.T., cryosectioned and immunostained for CD11b using M1/70 (BD Bioscience), for F4/80+ using BM8 (eBioscience) and for CD31 using MEC13.3 (BD Bioscience). Slides were counterstained with DAPI or TOPRO-3 (Invitrogen). Tissues were analyzed using Metamorph image capture and analysis software (Version 6.3r5, Molecular Devices). Haematoxylin and eosin staining was performed by the Moores UCSD Cancer Centre Histology Shared Resource. Metastases were quantified by immunostaining with Alexa488-conjugated murine anti-pan-cytokeratin (anti-cytokeratins 5, 6, 8, 10, 13, and 18, Clone C11) from Cell Signaling Technology. All experiments were performed 3 times. Data were analyzed for statistical significance with an unpaired two-tailed Student's t-test or analysis of variance (ANOVA) coupled with posthoc Tukey's test for multiple pairwise comparisons. P<0.05 was considered to be significant. Myeloid cells were quantified by immunohistochemical methods rather than by flow cytometry when insufficient material was available for quantification by flow cytometry.

Normal human mammary gland and invasive ductal breast carcinoma (n=10), 9 week old WT FVB and 9 week PyMT+FVB mouse mammary glands (n=6), normal mouse pancreata and d30 orthotopic Panc02 pancreatic tumours (n=6), normal mouse lungs and d12 orthotopic LLC carcinoma lung tumours (n=6), and normal mouse skin and d21 subcutaneous LLC tumours (n=6) were immunostained to detect CD11b+ cells using M1/70 (BD Bioscience).

### Quantification of Myeloid Cells in Tissues by Flow Cytometry

To quantify myeloid cells in tissues, tumours were excised, minced and digested to single cell suspensions for 2 h at 37° C. in 10 ml of Hanks Balanced Salt Solution (HBSS, GIBCO) containing 10 mg/ml Collagenase type IV (Sigma), 100 μg/ml Hyaluronidase type V (Sigma) and 200 units/ml DNase type IV (Sigma). Red blood cells were solublized with RBC Lysis Buffer (eBioscience) and then cells were incubated in FC-blocking reagent (BD Bioscience), followed by anti-CD11b-APC (M1/70, eBioscience) and anti-Gr1-FITC (RB6-8C5, eBioscience). To exclude dead cells, 0.5 μg/ml propidium iodide (PI) was added before data acquisition by FACs Calibur (BD Bioscience).

To quantify myeloid cells in murine peripheral blood, blood was collected from naïve or tumor-bearing mice by retro-orbital bleeding into heparin-coated Vacutainer tubes (BD Bioscience), incubated in red blood cell lysis buffer and stained with anti-CD11b-APC/Gr1-FITC.

CD11b+ Gr1+ myeloid cells from bone marrow or tumour tissue were further characterized by immunostaining to detect F4/80 (BM8-APC and -FITC), CD14 (Sa2-8-APC), cKit (ACK2-APC) and Tie2 (TEK4-PE) from eBioscience, as well as MHC-II (AF6 120.1), Ly6C (AL-21-FITC) and Ly6G (1A8-PE) both from BD Pharmingen. Data was acquired with a FACs Calibur instrument (BD Bioscience).

### Gene and Protein Expression

Total RNA was isolated from normal tissue, LLC tumours, Panc02 tumours, LLC and Panc02 cells as well as myeloid cells using ISOGEN (Nippon Gene). cDNA was prepared from 1 μg RNA from each sample and qPCR was performed using primers for Pi3ka, Pi3kb, Pi3kg, Pi3kd, Gapdh, Sdf-1α, IL-1β, TnfA, Il-6, αM (Cd11b), α4 and Cd31 from Qiagen (QuantiTect Primer Assay). qPCR for VegfA expression was performed with sense primers: 5′GCTGTGCAGGCTGCTCTAAC3′ and anti-sense primers: 5′CGCATGATCTGCATGGTGAT3′. Relative expression levels were normalized to gapdh expression according to the formula <2̂-^{(Ct gene of interest-Ct gapdh)}>³². Values were multiplied by 100 for presentation purposes. Fold increase in expression levels were calculated by comparative Ct method <2̂-^{(ddCt)}>³². Values for Panc02 were compared to normal pancreas and LLC to total cells isolated from subcutaneous implanted Growth Factor-depleted Matrigel. SDF-1α and IL-1β protein levels were determined in RIPA lysates of cultured tumour cells, whole tumours or CD11b+ cells purified from tumours using Quantikine mouse SDF-1α and IL-1β ELISA kits (R&D Systems).

Purification of Cells from BM or Buffy Coat

Human CD11b+ cells were purified from human buffy coats from the San Diego Blood Bank. Murine CD11b+ or Gr1+ cells were purified from murine BM by anti-CD11b or Gr1+ magnetic bead affinity chromatography according to manufacturer's directions (Miltenyi Biotec) or by fluorescence activated cell sorting. To assess the purity of the CD11b+ or Gr1+ cell population, allophycocyanin (APC) labelled anti-CD11b or Gr1 antibodies were added together with the magnetic beads and flow cytometry was performed.

### Adhesion Assays

1×10⁵ calcein-AM labelled human CD11b+ cells isolated from buffy coats from the San Diego Blood Bank or murine CD11b+ cells isolated from naïve (non-tumour bearing) or tumour-bearing mice were incubated on HUVEC monolayers or on plastic plates coated with 5 μg/ml recombinant soluble VCAM-1 or ICAM-1 (R&D Systems) for 30 minutes at 37° C. with humidity in the presence of LLC Tumour Conditioned Medium (TCM) or DMEM containing 200 ng/ml SDF1α, IL1β, IL6, TNFα or VEGF-A (R&D Systems). (SDF-1α and IL-1β were initially titrated to determine the minimum dose required to achieve near maximal stimulation of adhesion). TCM was prepared by incubating LLC cells in serum-free media for 18 h and filtering through 0.22 μm filters. After washing three times with warmed medium, adherent cells were quantified using a plate fluorimeter (GeniosPro, TECAN).

In some adhesion assays, cells were also incubated in 25 μg/ml function-blocking anti-integrin α4β1 (rat anti-murine, PS2 or mouse anti-human, HP2/1, gifts from Biogen-Idec), rat-anti-αM (anti-CD11b, M1/70) and isotype control antibodies (rat IgG2κ or mouse IgG1) or with 0.1 nM-10 μM doses of the small molecule inhibitor of integrin α4, ELN476063 (IC50=10 nM), a gift from Elan¹⁴.

Additionally, labelled cells were incubated with HUVEC and rsVCAM coated plates in the presence of 200 ng/mL-1β or SDF-1α and 1-10 μM inhibitors: pan-PI3 kinase inhibitors (LY2942002, wortmannin), PI3-kinase α inhibitors (PI3K75, PIK2alpha, PI103), PI3-kinase β inhibitor (TGX221), PI3-kinase γ inhibitors (TG100-115, AS605240, AS604850), inert control, PLC inhibitor (U73122), geranylgeranyltransferase (Rap1-selective) inhibitors (GGTI-2147 and GGTI-298) or farnesyltransferase (Ras-selective) inhibitor (FTI-277), Akt inhibitors (Inhibitor X and peptide 18), mTOR inhibitor (rapamycin), ROCK inhibitor (Y27632), MEK inhibitor (PD98059), p38 inhibitor (SB202190), tyrosine kinase inhibitor (genestein) and PKA inhibitors (H89, KT5720). LY294002, wortmannin, genestein, U73122, GGTI-2147, GGTI-298, FTI-277, Y27632, PD98059, SB202190, H89 and KT5720 were from Calbiochem. PIK2alpha, PI103, AS605240, AS604850 and TGX221 were from Echelon. TG100-115 was prepared as described²⁵⁻²⁶. To titrate the effect of various signalling protein inhibitors on myeloid cell adhesion stimulated by IL-1β or SDF-1α, cells were incubated in 1 nm to 100 μM PI3-kinase inhibitors TG00020 (a pan-PI3-kinase inhibitor), PI3-kinase α inhibitor (PI3Kalpha2) PI3-kinase β inhibitor (TGX221), PI3-kinase γ inhibitors (TG100-115, AS605240), an inert chemically matched control, farnesyltrasnferase inhibitors and geranylgeranyltransferase inhibitors. IC₅₀s for GGTI and FTI were 1 μM. IC₅₀s for PI3K inhibitors are reported in Supp. FIG. 11a,c. For adhesion assays with plasmid-transfected cells, cells were serum starved for 4 h, and then incubated for 20 min on chamber-slides coated with 5 μg/ml rsVCAM-1. Adherent GFP+ cells were automatically quantified using MetaMorph software (Molecular Devices Software).

Additionally, CD11b+ cells from WT, α4Y991a, α4−/− (Tie2Creα4^{loxp/loxp}), and p110γ−/− mice were stimulated with chemokines and incubated with HUVEC- or VCAM-1-coated plates. Gr1+myeloid cells from αM−/− (Cd11b−/−) and WT mice were also stimulated and incubated with HUVECs and VCAM-1 coated plates. As expected, no differences were observed in the degree of adhesion of Gr1+WT and CD11b+ WT myeloid cells.

CD11b+Gr1+ cells were isolated from bone marrow of naïve or LLC tumour bearing WT mice, stimulated with chemokines and incubated with HUVEC- or VCAM-1-coated plates.

### Sorting of Integrin Alpha 4−/− Bone Marrow Cells.

Bone marrow derived cells were isolated from Tie2Cre+ integrin α4^{loxp/loxp} mice were incubated with anti-CD11b-APC, anti-CD49d-FITC and 0.5 μg/ml propidium iodide. CD11b+CD49d+PI− and CD11b+CD49−PI− cells were collected using Aria FACs sorting at the Moores Cancer Center Shared Resource. Cells were used in adhesion and in vivo homing assays.

### Ligand (VCAM-1) Binding Assay

5×10⁵ CD11b+ cells isolated from WT, α4Y991A, or PI3-kinase γ−/− mice (naïve or tumour-bearing) were incubated with 200 ng/ml IL-1β, SDF-1α, IL-6, TNFα, VEGF-A or medium together with 1 mg/ml mouseVCAM-1/humanFc fusion protein (R&D Systems) for 3 min. Cells were washed twice and incubated with donkey anti-human-FC-PE antibody (Jackson Immunoresearch) then analysed by FACs Calibur. Mean fluorescence intensity of treated cells was compared to that of unstimulated cells (basal). In other studies, cells from WT mice were untreated or were treated with 1 μM PI3-kinase α, β, or γ inhibitors for 30 min at 37° C. In some experiments GFP/RapV12 or GFP/RasV12 transfected CD11b+ cells were used. In this case, increased mean fluorescence intensity was measured for only GFP-positive cells.

### Analysis of Integrin Expression and Activation

Expression levels of murine integrin α4 on CD11b+ cells were determined by flow cytometry for PE-conjugated R1/2 (rat anti-CD49d antibody, eBioscience). Integrin α4 levels on human CD11b+ cells were determined by flow cytometry for HP2/1 (anti-human α4 antibody, Biogen Idec).

The activation state of β1 integrins (CD29) on human CD11b+ cells was quantified by flow cytometry using HUTS-21 (an anti-β1 integrin activation induced epitope antibody, BD Bioscience) and total β1 integrin levels were assessed using P4C10 antibodies (Chemicon) as follows. 2.5×10⁶ freshly isolated human myeloid cells/ml were incubated in culture medium containing 10 μg/ml normal human immunoglobulin (12000C, Caltag) for 45 min on ice. These cells were then incubated in 200 ng/ml SDF-1α, IL-1β, IL-6 or 1 mM Mn2+ plus 2.5 μg HUTS21, P4C10, or IgG2 control for 10 min at 37° C., followed by Alexa 488 goat-anti mouse antibodies for 20 min on ice.

### Drug Treatment of Tumours

IL-1β, SDF-1α and integrin α4 inhibitor studies: C57B16 mice were subcutaneously implanted on d1 with 1×10⁶ LLC cells and were treated on d3 and d5 with intraperitoneal injections of function-blocking anti-IL-1β antibodies (MAB401 from R&D Systems) (n=16) or isotype-matched control antibodies rat IgG1, (n=14) (100 μg/25 g body weight). Mice were sacrificed on d7. In alternative studies, mice were treated by i.p. injection with saline (n=6) or 1.25 mg/kg SDF-1α inhibitor (AMD3100, Sigma Aldrich) (n=7) daily for eight days, starting one day prior to subcutaneously implantation of 1×10⁶ LLC cells. In further studies, mice were treated with anti-IL-1β and SDF 1α inhibitor. In other studies, C57BL/6 mice were subcutaneously implanted on d1 with 0.5×10⁶ LLC cells and were treated every third day with subcutaneous injections of ELN476063 (3 mg/kg body weight), an integrin α4 small molecule inhibitor.

Alternatively, 6 week old PyMT+ female mice (with spontaneous breast tumours), were treated by subcutaneous injection with ELN476063 (3 mg/kg body weight), or saline every third day for three weeks (n=10). Tumours were excised, weighed and analyzed from 6 weeks at 9 weeks of age.

PI3-kinase γ inhibitor studies: C57BL/6 mice were subcutaneously implanted on d1 with 5×10⁵ LLC or by intradermal injection with 5×10⁵ B16 melanoma cells. Mice were treated by i.p. injection with 2.5 mg/kg of PI3-kinase γ inhibitor (TG100-115) or with a chemically similar inert control (n=10) twice daily for fourteen days for a total daily dose of 5 mg/kg. In additional studies, C57BL/6 mice were subcutaneously implanted on d1 with 5×10⁵ LLC and were treated by i.p. injection with 2.5 mg/kg, 0.25 mg/kg, or 0.025 mg/kg of PI3-kinase γ inhibitor (TG100-115), with 2.5 mg/kg of AS605240 or with a chemically similar inert control (n=10) twice daily for twenty-one days for a total daily dose of 5 mg/kg. Tumour volumes, weights and blood vessel densities, as well as myeloid cell densities were measured. Alternatively, 6 week old PyMT+ female mice (with spontaneous breast tumours), were treated by i.p. injection with 2.5 mg/kg PI3-kinase γ inhibitor (TG100-115) or inert control twice daily for three weeks (n=10). Tumours were excised, weighed and analyzed after 3 weeks. Alternatively, 6 week old FVB PyMT+ female mice were implanted with an Alzet osmotic pump with a 0.25 μl/h release rate containing 4.6 mg in 200 μl of TG100-115 or chemically similar, inert control (n=10). Mice were sacrificed at 9 weeks of age and tumours were analyzed.

Pharmacokinetic analysis of a 5 mg/kg bolus dose TG100-115 in Balb-c mice was accomplish by evaluating TG100-115 levels in serum at 5, 15, 30, 60 minute intervals as was previously performed for rats²⁵. In vivo activity assay of a 5 mg/kg bolus dose TG100-115 in Balb-c mice was accomplish by evaluating pAkt/Akt levels in peripheral blood cells in response to SDF-1α stimulation at 0.25, 0.5, 1, 2, 4, 6 and 12 hour intervals

### Mammary Gland Whole Mounts

Inguinal mammary glands were fixed in Carnoy's fixative, dehydrated through a graded series of ethanol solutions and defatted in xylene. Following rehydration, the mammary epithelium was stained with carmine stain (Sigma Chemical, St Louis, Mo.) for 30 min. After removing excess stain by washing in water, samples were dehydrated and stored in methyl salicylate (Sigma Chemical, St Louis, Mo.).³¹

### Isolation of Bone Marrow Derived Cells for Bone Marrow Transplantation

Bone marrow derived cells (BMDCs) were aseptically harvested from 6-8 week-old female mice by flushing leg bones of euthanized mice with phosphate buffered saline (PBS) containing 0.5% BSA and 2 mM EDTA, incubating cells in red cell lysis buffer (155 mM NH₄Cl, 10 mM NaHCO₃ and 0.1 mM EDTA) and centrifuging over Histopaque 1083. Approximately 5×10⁷ BMDC were purified by gradient centrifugation from the femurs and tibias of a single mouse. Two million cells were intravenously injected into tail veins of each lethally irradiated (1000 rad) 6 week old syngeneic recipient mouse. After 4 weeks of recovery, tumour cells were injected in BM transplanted animals. LLC (n=8, 3 experiments) and Panc02 (n=8, 2 experiments) tumour growth in C57BL/6 and α4Y991A mice transplanted with BM from α4Y991A or WT were compared as described above.

### PI3-Kinase Activation in Myeloid Cells.

CD11b+ cells from C57BL/6 mice or PI3-kinase γ−/− mice were freshly isolated under serum free-conditions and were incubated for 30 min in serum-free media in the presence or absence of 1 μM TG100-115. CD11b+ cells were then stimulated with 200 ng/ml IL-1β or SDF-1α (R&D Systems) for 1-3 minutes and cells were solubilized with RIPA buffer. Alternatively, mice were injected intravenously with 2.5 mg/ml TG100-115 or a chemically similar inert control. Mononuclear peripheral blood leukocytes were isolated from 2 mice each at 0.5, 1, 2, 4, 6 and 12 hours after treatment. Cells were treated for 3 minutes with 200 ng/ml SDF and then solubilized with RIPA buffer. Lysates were electrophoresed and Western blotted. Akt phosphorylation was evaluated by immunoblotting with anti-phosphorylated Thr308-Akt specific antibody (C31 E5E, Cell Signalling). Blots were stripped and reprobed with anti-Akt (#9272, Cell Signaling). Films were scanned and quantified by densitometry.

### Rap1 Activity Assay

Total BM was isolated from WT mice under serum free conditions. BM cells were incubated for 30 min at 37° C. in serum free media in the presence or absence of 1 μM PI3-kinase γ inhibitor (TG100-115), followed by stimulation with basal medium or medium containing 200 ng/ml IL-1β, IL-6 or SDF-1α (R&D Systems). Cells were lysed and activated Rap1 was pulled down from 1 mg cell lysate after addition of RalGDS Rap1-binding domain-GST fusion proteins and glutathione-conjugated beads. Beads were boiled in SDS sample buffer and electrophoresed on SDS gels. Activated (pulled down) Rap1 was detected by immunoblotting with anti-Rap1 antibodies (from Rap1 pulldown assay kit, Thermo Scientific, 89872).

### Immunoprecipitation of Integrin α4 and Associated Proteins

BM cells (comprised of 80% CD11b+Gr1+ cells) from WT or α4Y991A mice were isolated as described above and treated with either DMEM or TCM for 30 min at 37° C. Cells were rinsed with cold PBS and lysed in Tris-buffered saline containing 1% CHAPS, 20 mM β-glycerophosphate, 1 mM Na₃VO₄, 5 mM NaF, 100 ng/ml microcystin-LR, and protease inhibitor cocktail. After centrifugation, integrin α4 in cell lysates were immunoprecipitated as follows: 1 mg total protein was precleared with 10 μl protein G-conjugated Dynabeads (Invitrogen) for 1 hr at 4° C. with rotation. Cleared lysates were incubated with 5 μg of rat anti-α4β1 (PS/2) antibody at 4° C. overnight. 25 μl of protein G-conjugated Dynabeads was then added for 3 h with rotation. Beads were washed three times with 1 ml cold PBS containing protease inhibitor cocktail. Protein precipitates were electrophoresed on 10% SDS-PAGE gels and immunoblotted with anti-integrin α4 (C-20, Santa Cruz Biotechnology), anti-talin (Clone TD77, Chemicon) or anti-paxillin (H-114, Santa Cruz Biotechnology) antibodies. Immune complexes were visualized using an enhanced chemiluminescence detection kit (Pierce).

siRNA Mediated Knockdown

Freshly isolated CD11b+ cells from mouse BM were transfected using an AMAXA Mouse Macrophage Nucleofection Kit with 100 nM of siRNA for Rap1 (Mm_Rap1a_{—}1 & Mm_Rap1a_{—}7), Nras (Mm_Nras_{—}2 & Mm_Nras_{—}3), Hras (Mn_Hras1_{—}1 & Mm_Hras1_{—}2), Kras (Mm_Kras2_{—}1 & MmKras2_{—}3), PI3Kα (Mm_pik3ca_{—}1 and Mm_pik3ca_{—}3), PI3Kβ (Mm_pik3cb_{—}2 and Mm_pik3cb_{—}4), PI3kγ (Mm_pik3cg_{—}1 and Mm_pik3cg_{—}2), PI3Kδ (Mm_pik3cd_{—}1 and Mm_pik3cd_{—}2), itga4 (Mm_itga4_{—}1 & Mm_itga4_{—}2) itgam ((Mm_itgam_{—}1 & Mm_itgam_{—}5) or non-silencing siRNA (Ctrl_AllStars_{—}1) purchased from Qiagen. After transfection, cells were cultured for 36-48 h in media containing 20% serum. Each siRNA was tested individually for efficient knockdown of protein expression and for inhibition of adhesion. qPCR Primers used to validate mRNA levels included Mm_pik3ca_{—}1_SG, Mm_pik3cb_{—}1_SG, Mm_pik3cg_{—}1_SG, Mm_pik3cd_{—}1_SG, Mm_ita4_{—}1_SG, and Mm_itam_{—}1_SG from Qiagen. Antibodies used to validate protein levels included PI3K alpha (#42550), beta (#3011), gamma (#4252) from Cell Signaling. To validate integrin expression cells were analyzed by flow cytometry using anti-α4 (R1-2) and anti-αM (M1/70) antibodies from eBioscience. Similar results were achieved for each siRNA oligo listed above. Results are presented for Mm_Rap1a_{—}1, Mm_Nras_{—}2, Mm_Kras2_{—}1, Mm_pik3ca_{—}1, Mm_pik3b_{—}2, Mm_pik3cg_{—}1, Mm_pik3cd_{—}1, Mm_itga4_{—}1, and Mm_itgam_{—}1.

### RapV12 and RasV12 Transfection

Plasmids expressing constitutively active Rap (pRapV12)⁷ or Ras (pRasV12)⁸ were co-transfected with pGFP using an AMAXA Mouse Macrophage Nucleofection Kit into CD11b+ cells from WT and PI3-kinase γ−/− mice and were cultured for 36 h in media+20% serum. Transfection efficiency was around 30-40% as determined by GFP-FACs. Constructs were tested for ability to induce adhesion in the absence of stimulation and in the presence of TG100-115 (1 μM). In some studies cells were co-transfected with constitutively active Rap (pRapV12) or Ras (pRasV12) and siRNA directed against PI3Kγ or integrin α4.

### In Vivo Myeloid Cell Trafficking Studies

CD11b+ cells from C57BL/6 mice were fluorescently labelled with carboxyfluorescein succinimidyl ester (CFSE, 5 μM, Invitrogen), and CD11b+ cells from α4Y991A mice were labelled with cell tracker red (5 μM CMTPX™, Invitrogen). Cell viability was tested with Trypan blue staining and cell functionality was tested by adhesion assays in vitro. Green- and red-labelled cells were mixed 1:1 and 4×10⁶ cells were injected intravenously into the tail vein of mice bearing 1-week-old LLC carcinomas grown under dorsal skin-fold window chambers. Accumulated fluorescent cells were visualized with live animal confocal microscopy and were quantified one hour after injection.

Alternatively, 5×10⁶ CFSE labelled CD11b+ cells from C57BL/6, α4Y991A, and PI3-kinase γ−/− mice were injected intravenously into mice bearing subcutaneous (5×10⁵) d14 LLC tumours. In other studies, CD11b+ cells from WT mice were treated for 1 h with 1 μM PI3-kinase α inhibitor (PI3K75), PI3-kinase β inhibitor (TGX221), PI3-kinase γ inhibitor (TG100-115) and an inert control or with 10 μM PLC inhibitor (U73122), geranylgeranyltransferase inhibitor (GGT2147) or farnesyltransferase inhibitor (FTI-277) and then injected intravenously into mice bearing subcutaneous (5×10⁵) d14 LLC tumours.

In other studies, K, N and H Ras, Rap1, PI3Kα, PI3Kβ, PI3Kγ, PI3Kδ, integrin α4 and integrin αM were knocked down by siRNA transfection of CD11b+ cells as described above. 48 h later, cells were labeled with CFSE and injected into mice with d14 LLC tumours (1×10⁶ cells). Fluorescent cells accumulating in tumours and spleens were quantified 2 h and 24 h later by excising tissues, preparing single cell suspensions and performing FACs analysis at 488 nm. No differences were observed between untreated and non-silencing siRNA treated cells.

### In Vivo Angiogenesis Assays

Growth Factor-depleted Matrigel (BD Bioscience) containing 400 ng SDF-1α, IL-1β (R&D Systems) or saline in 400 μl was injected subcutaneously into C57BL/6 mice (n=6) transplanted with BM from beta-Actin EGFP+ mice (from Jackson Laboratories). One week later, Matrigel plugs were excised, cryopreserved, sectioned and immunostained for the presence of myeloid cells and blood vessels. In additional studies, 400 μl Growth Factor-depleted Matrigel (BD Bioscience) containing 400 ng bFGF (R&D Systems) or saline was injected subcutaneously into C57BL/6 WT or PI3-kinase γ −/− mice. After 5 days, mice were injected intravenously with 20 μg FITC-conjugated Bandeira simplicifolia lectin-I (Vector Laboratories), Matrigel plugs were removed and homogenized. Fluorescence was quantified at 520 nm using a fluorimeter (Tecan).

### Cell Proliferation Assay.

Two thousand LLC or PyMT breast carcinoma cells were seeded into 96 well plate wells in the presence or absence of 0.1-10 μM Pan-PI3-kinase inhibitor (TG00020), PI3-kinase γ inhibitor (TG100-115) or inert control. Cell proliferation was measured after 24 h, 48 h, and 72 h according to the manufacturer's protocol (WST-1 cell proliferation reagent, Roche).

### Statistics

Statistical significance was determined by ANOVA coupled with posthoc Tukey's test for multiple pairwise comparison,

### Example 10

### Results Obtained Using the Methods of Example 9

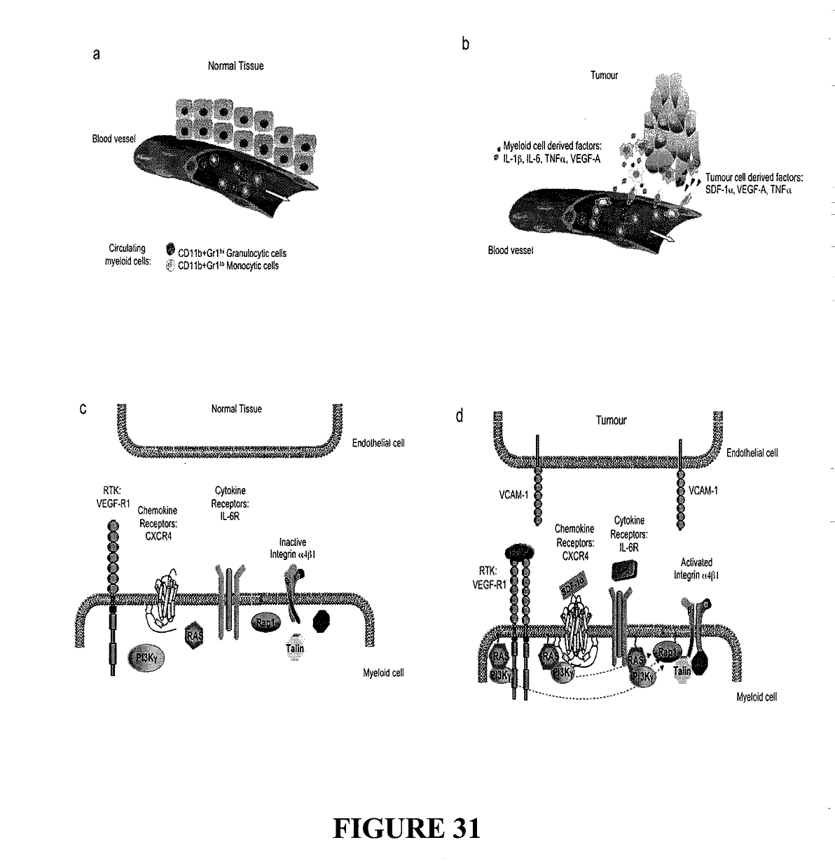

Cancer and inflammation are linked, as chronic inflammatory diseases increase the risk of developing tumours¹, and growing tumours induce host inflammatory responses that stimulate tumour progression²⁻⁶. Myeloid cells, including granulocytes, monocytes, myeloid-derived suppressor cells and tumour-associated macrophages, invade the tumour microenvironment in response to a variety of chemoattractants and promote tumour angiogenesis^{3,5-9}, immunosuppression^{2,4,10-11} or metastasis¹². We show here that a single, common Ras-PI3-kinase γ-integrin α4β1 pathway regulates myeloid cell extravasation, tumour inflammation and tumour progression, regardless of the specific chemoattractant produced by the tumour. Chemoattractants released from tumour cells, including SDF-1α, TNFα and VEGF-A, and those released from tumour macrophages, such as IL-1β and IL-6, stimulate Ras, PI3-kinase γ and Rap1a-dependent integrin α4β1 activation and integrin α4β1-dependent recruitment of myeloid cells to the tumour microenvironment. Genetic or siRNA mediated ablation or chemical inhibition of N- and K-Ras, PI3-kinase γ, Rap1a or integrin α4 function blocks myeloid cell adhesion to vascular endothelium and recruitment to implanted or spontaneous tumours, leading to a reduction in tumour angiogenesis, growth and metastasis. These findings help to define further the role that myeloid cells play in establishing chronic inflammation in cancer and indicate for the first time that use of inhibitors of PI3-kinase γ or integrin α4β1 represents an innovative approach to control tumour malignancy. (These results are summarized in FIG. 31).

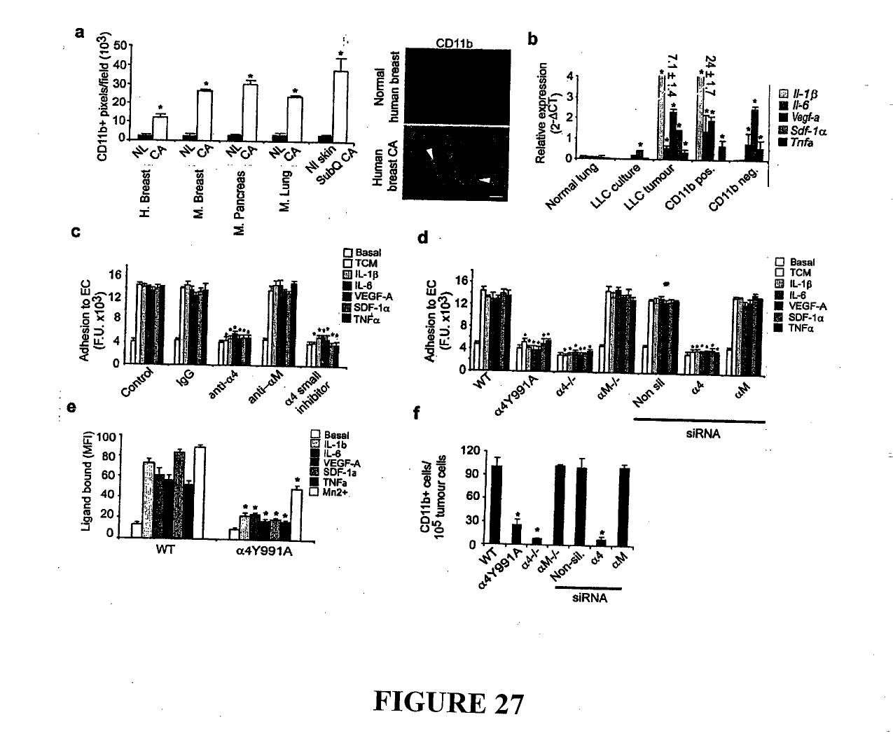

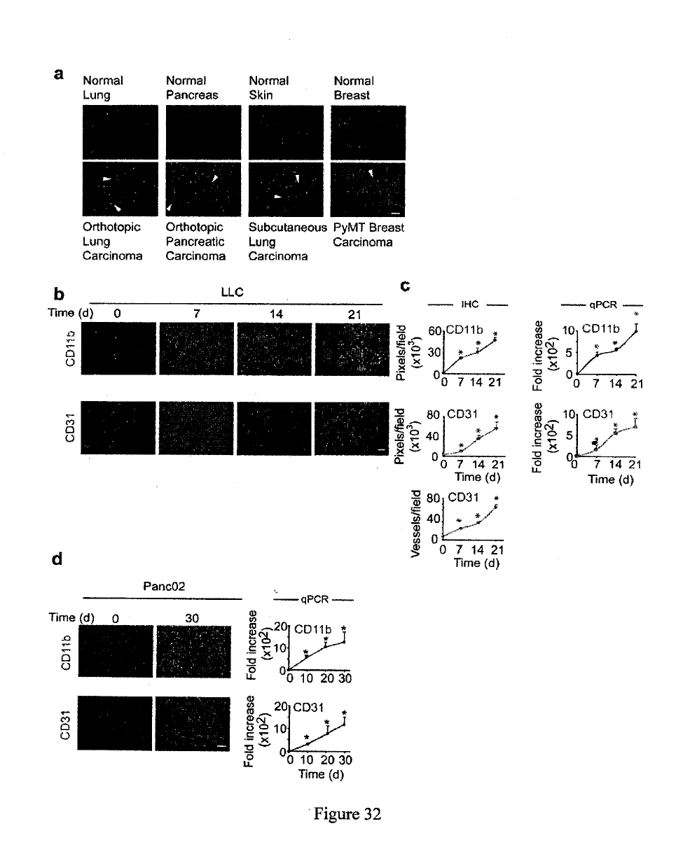

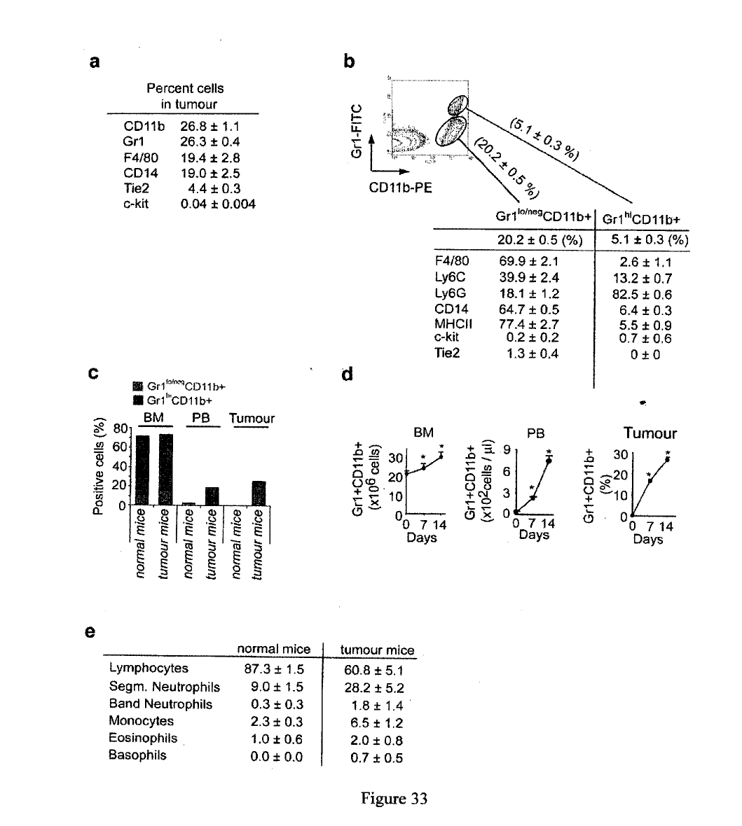

To identify pathways that regulate tumour inflammation, we characterized myeloid cell recruitment to human and murine tumours. CD11b+ myeloid cells extensively and persistently invaded tumours but not normal tissues (FIGS. 27,32a-d). Myeloid cell invasion was proportional to angiogenesis, supporting a role for myeloid cells in tumour angiogenesis (FIG. 32b-d)^{3,5-9}. These cells comprised one quarter of the tumour mass and consisted of 20% Gr1^{hi}CD11b+ neutrophils and 80% Gr1^{lo}CD11b+ monocytic-lineage cells, which were primarily Gr1^{lo}CD11b+F4/80+CD14+MHCII+ macrophages (FIG. 33a-c). In contrast, myeloid cells in peripheral blood (PB) and bone marrow (BM) of both normal and tumour-bearing animals were comprised of 80% Gr1^{hi}CD11b+ neutrophils and 20% Gr1^{lo}CD11b+ monocytes (FIG. 33c-e); the absolute numbers but not relative proportions of these cells increased during tumour development (FIG. 33d-e). These results indicate that tumours rapidly and persistently recruit pro-angiogenic myeloid cells from PB and BM.

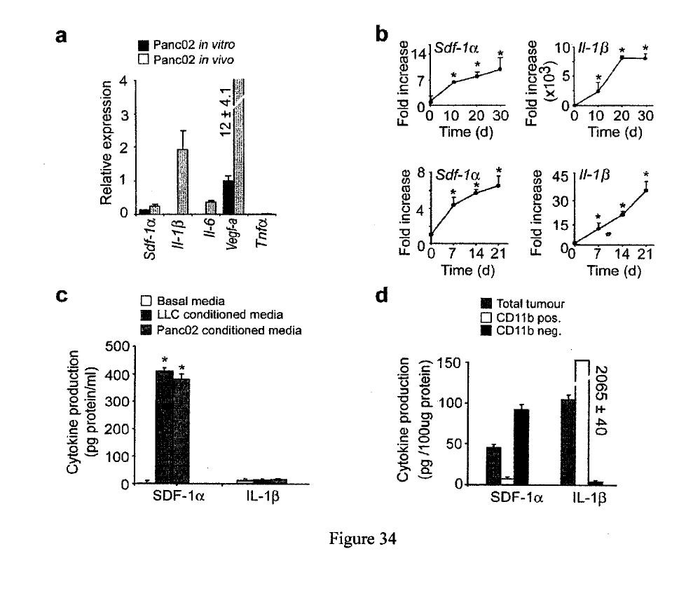

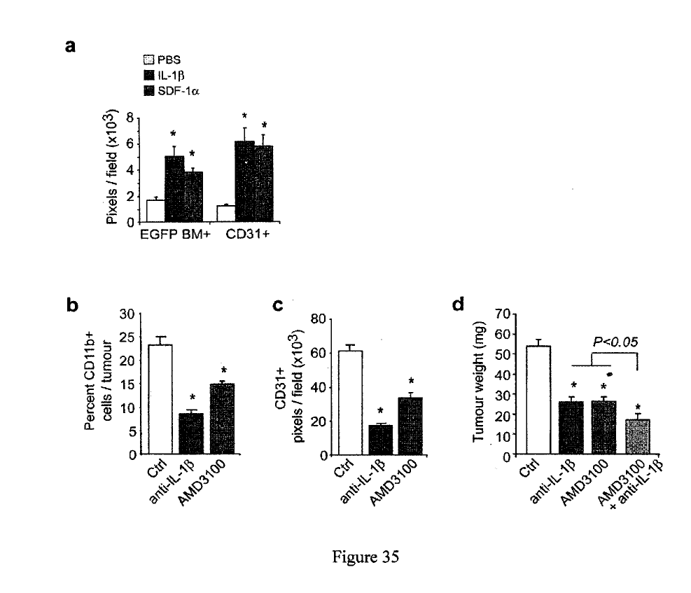

To determine whether specific chemoattractants recruit myeloid cells to the tumour microenvironment, we examined the expression profiles of chemoattractants in tumours. Tumours but not normal tissues expressed Sdf-1α, Vegf-A, Tnfα, IL-1β and IL-6 from the earliest stages of growth (FIGS. 27b, 34a-d). Tumour myeloid cells were the exclusive source of IL-1β and IL-6, while tumour cells were the exclusive source of Sdf-1α (FIGS. 27b, 34a-d). Myeloid cell-derived factors, such as IL-1β, and tumour-derived factors, such as SDF-1α, each promoted myeloid cell recruitment and subsequent angiogenesis in vivo (FIG. 35a); selective antagonists of these factors, alone or together, suppressed myeloid cell invasion, angiogenesis and tumor growth (FIG. 35b-d). These results indicate that blockade of tumour and myeloid cell-derived chemoattractants may provide anti-tumour therapeutic benefit by suppressing inflammation. However, as most tumours produce multiple chemoattractants¹⁻¹¹, we sought to determine whether a common mechanism could regulate myeloid cell recruitment to tumours.

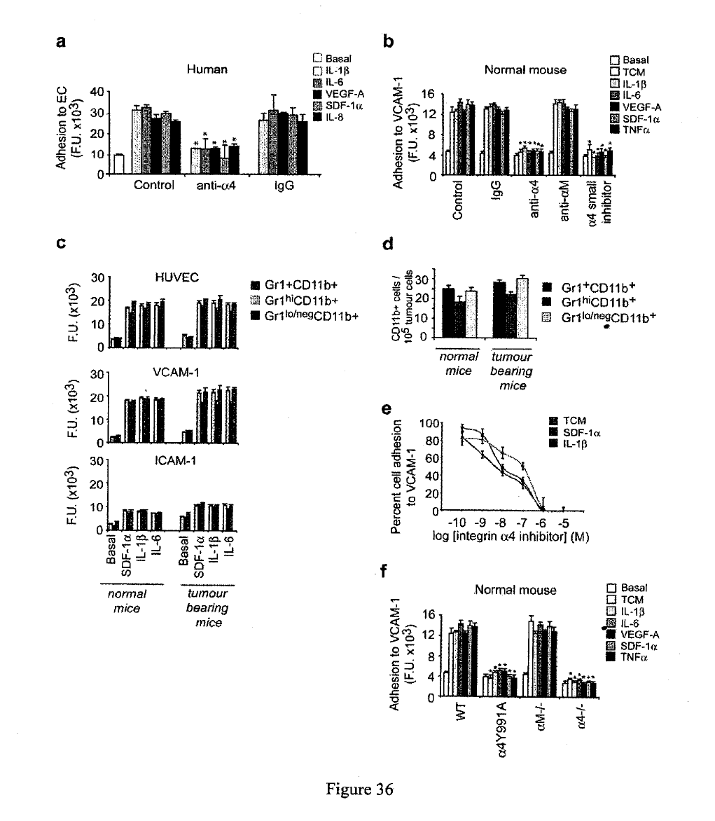

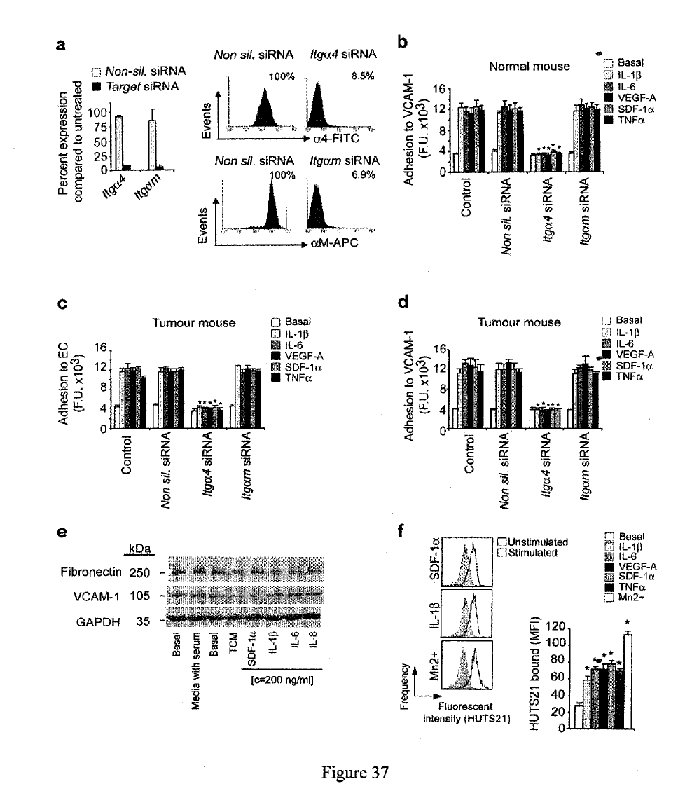

As immune cell extravasation can depend on integrin-mediated adhesion to endothelial cell (EC) counter-receptors VCAM-1 or ICAM-1¹²⁻¹³, we tested the ability of chemoattractants to promote myeloid cell adhesion to endothelium. Human CD11b+ cells and murine CD11b+, Gr1^{hi}CD11b+ and Gr1^{lo}CD11b+ cells from normal and tumour-bearing mice adhered strongly to EC and VCAM and slightly to ICAM in response to diverse chemoattractants (FIGS. 27c, 36a-c) and arrested in LLC tumours upon adoptive transfer into mice (FIG. 36d). As myeloid cells express the VCAM-1 and ICAM-1 receptors, integrins α4β1 and αMβ2 (Mac-1, CD11b), we tested antagonists of these integrins on cell adhesion¹⁴⁻¹⁵. Inhibitors of α4β1, but not αMβ2, suppressed myeloid cell adhesion to EC and VCAM-1 (FIGS. 27c, 36a-b, 36e). Cells with inactive integrin α4 (from α4Y991A mice, which exhibit decreased lymphocyte adhesion and invasion in vivo¹⁶⁻¹⁷), deleted integrin α4 (α4−/−, isolated from Tie2Cre+α4fl/fl mice¹⁸), and siRNA ablated integrin α4 failed to adhere to EC or VCAM-1 (FIGS. 27d, 36f, 37a-d), while αM−/− and αM siRNA-transfected cells adhered normally (FIGS. 27d, 36f, 37a-d). As chemoattractants had no effect on EC expression of VCAM-1 or fibronectin (FIG. 37e) or myeloid cell integrin expression during the assay period, chemoattractants stimulate myeloid cell adhesion by increasing integrin activity^{16-17,19}.

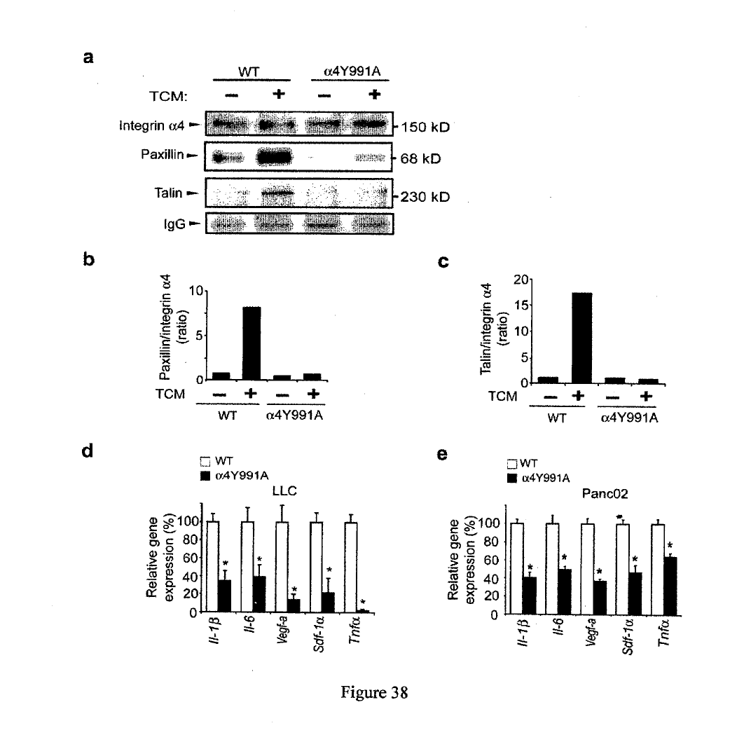

Extracellular stimuli can induce integrin conformational changes that result in increased ligand-binding and cell adhesion¹⁹. Tumour-derived chemoattractants rapidly stimulated ligand-binding to WT but not integrin α4Y991A myeloid cells (FIG. 27e) and induced integrin 61 conformational changes, as measured by cell surface binding of HUTS21²⁰, an antibody that recognizes an epitope expressed only on activated human β1 integrin (FIG. 37f). Chemoattractants also stimulated association of talin and paxillin¹⁹ with integrin α4 from WT but not α4Y991A cells (FIG. 38a-c), indicating that diverse chemoattractants activate integrin α4β1.

To determine whether myeloid cell recruitment to tumours depends on integrin α4 activity, we adoptively transferred fluorescently labelled WT, α4Y991A, α4−/−, αM−/−, as well as α4 and αM siRNA transfected myeloid cells, into tumour-bearing WT mice. Myeloid cells with defective or ablated integrin α4β1 failed to arrest in tumours, while cells with ablated αM infiltrated tumours normally, providing evidence that integrin α4, but not αM, is required for myeloid cell tumour infiltration in vivo (FIG. 27f).

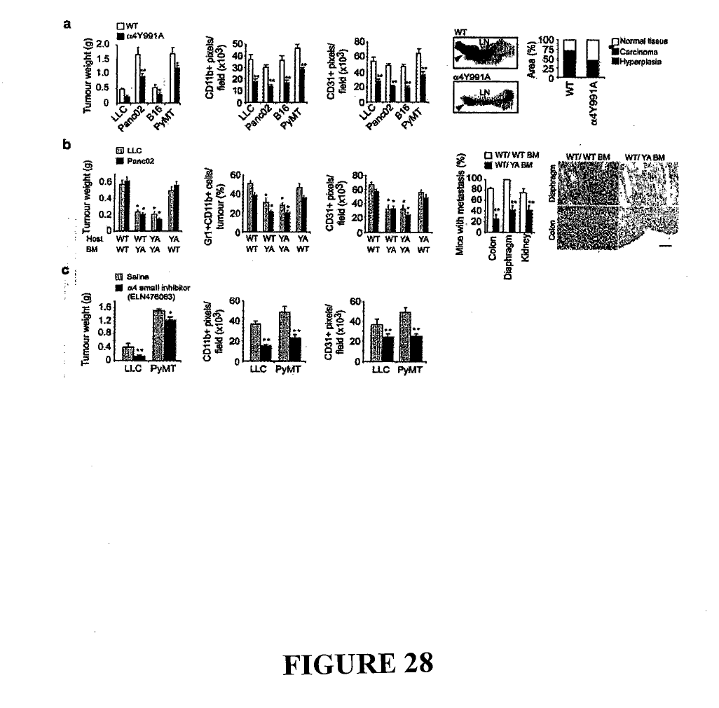

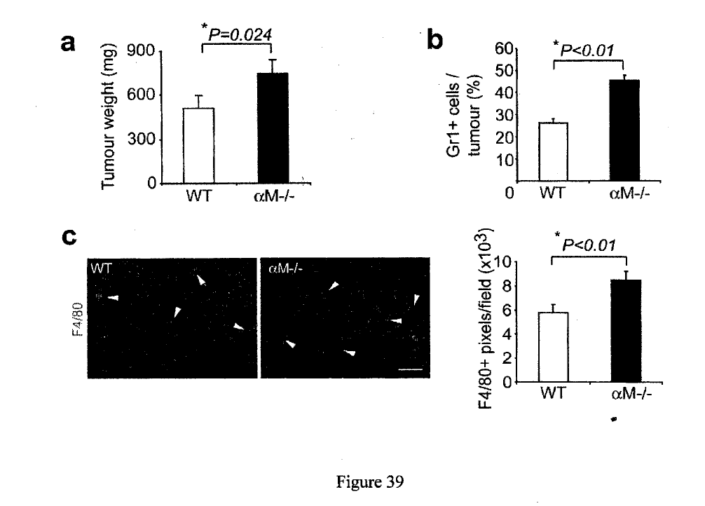

To evaluate the contribution of myeloid cell trafficking to tumour growth in vivo, we characterized the growth of subcutaneous lung carcinoma (LLC), orthotopic pancreatic carcinoma (Panc02), orthotopic B16 melanoma and spontaneous PyMT breast carcinoma in WT and α4Y991A mice. Myeloid cell infiltration, neovascularization, tumour growth and expression of pro-angiogenic and inflammatory factors were strongly suppressed in α4Y991A animals (FIGS. 28a, 38d-e). In addition, α4Y991A PyMT breast tumour progression was inhibited, as more tumours were limited to the hyperplastic stage (FIG. 28a). In contrast, tumour growth, inflammation and angiogenesis were not suppressed in αM−/− animals (FIG. 39a-c), indicating that only integrin α4 regulates tumour inflammation and associated angiogenesis and growth.

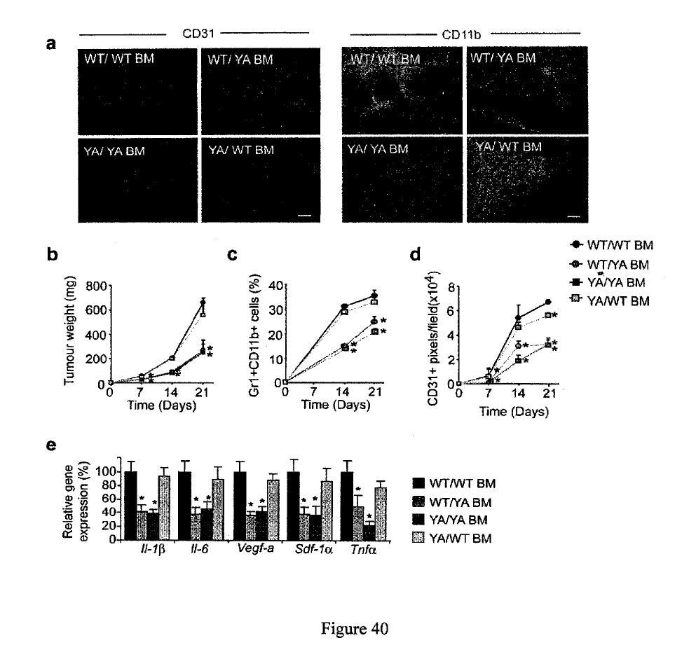

To confirm that tumour suppression in α4Y991A mice results from defects in BM-derived cell trafficking, we implanted subcutaneous LLC and orthotopic pancreatic tumour cells into BM-chimeric animals. Recruitment of Gr1+CD11b+myeloid cells, angiogenesis, tumour growth, spontaneous metastases and the expression of pro-angiogenic and inflammatory factors (FIG. 28b; FIG. 40a-e) were suppressed in WT or α4Y991A mice transplanted with α4Y991A BM, but not in WT or α4Y991A mice with WT BM. Because there were no differences in the numbers of myeloid cells in PB or BM of α4Y991A and WT animals, in their abilities to differentiate into macrophages or to stimulate angiogenesis (not shown), these studies indicate that integrin α4 activation in myeloid cells promotes tumour inflammation and growth.

We determined whether integrin α4 antagonists could also block tumour inflammation and growth. Treatment of mice with LLC and PyMT spontaneous breast tumours with the small molecule antagonist of integrin α4β1, ELN476063¹⁵, substantially suppressed tumour inflammation, angiogenesis and growth (FIG. 28c). Together, these studies demonstrate that α4β1 inhibitors could be useful to suppress tumour inflammation and growth.

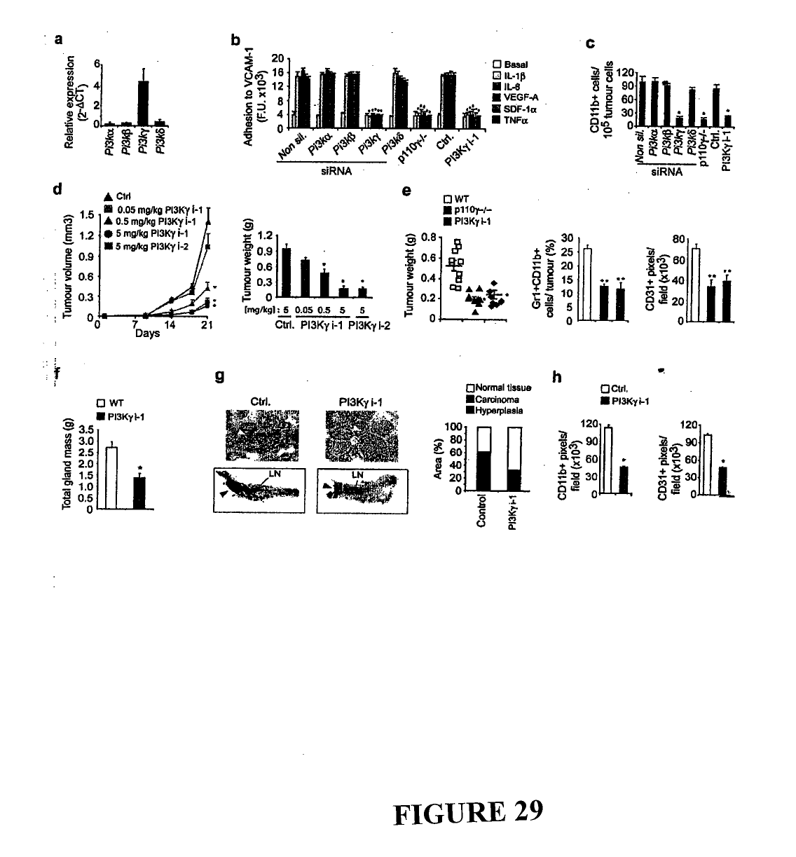

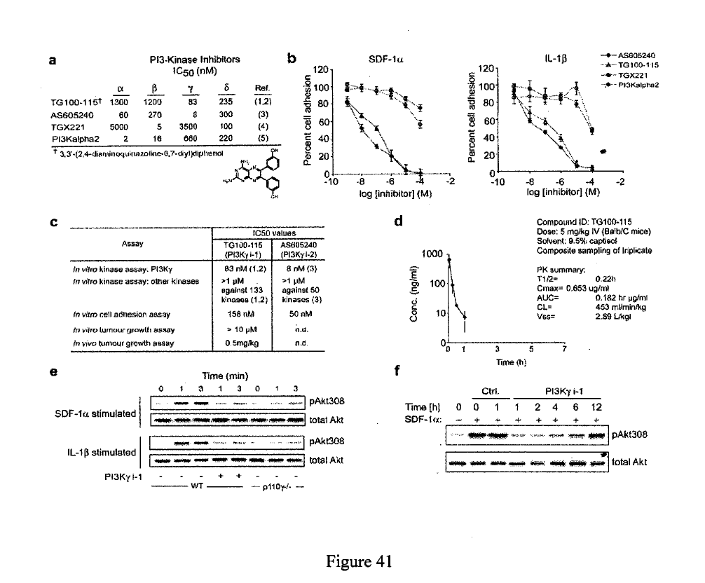

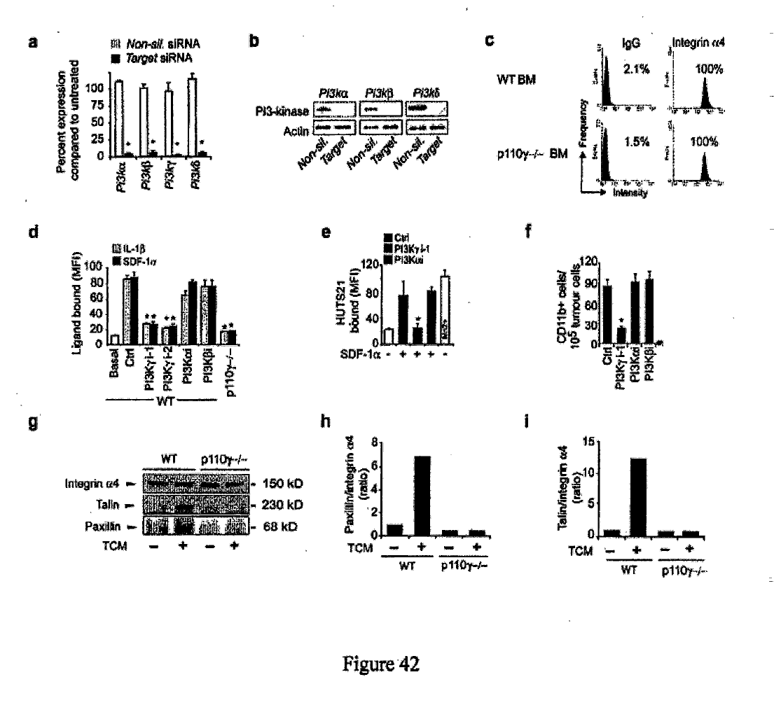

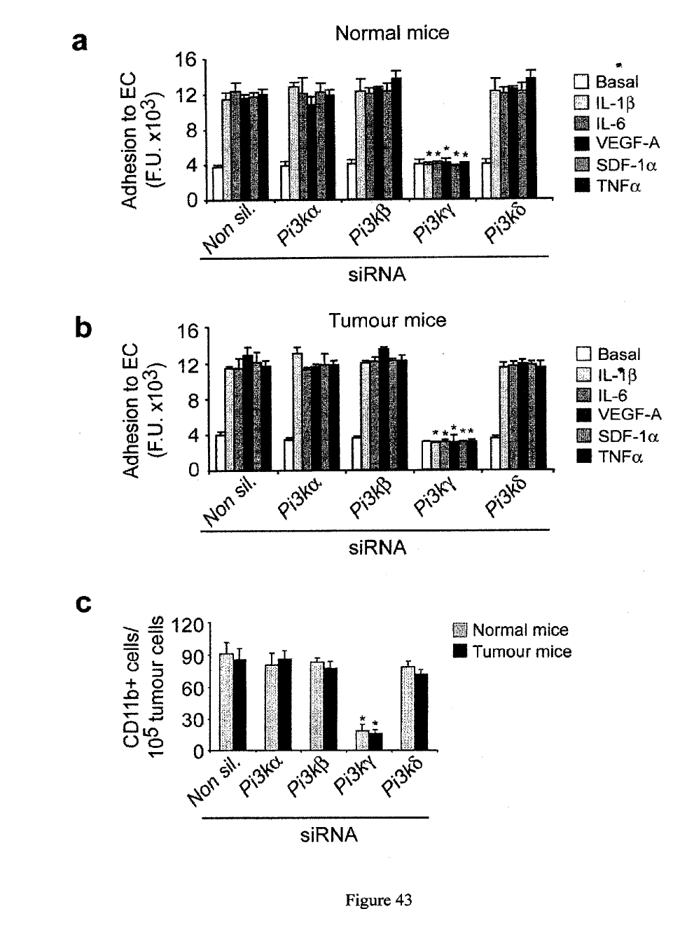

SDF-1α, VEGF-A, IL-1β, TNFα and IL-6 activate structurally diverse (G-protein coupled, Type III tyrosine kinase, Toll-like and type I cytokine) receptors, yet each activates integrin α4β1, suggesting a common signalling pathway. To identify such a pathway, we screened signalling inhibitors in myeloid cell adhesion assays and found that inhibitors of PI3-kinase γ but not tyrosine kinases, ROCK, ERK, p38, PLC, Akt, mTOR or PKA (not shown) suppressed myeloid cell adhesion. In fact, PI3-kinase γ has been implicated in neutrophil and thymocyte chemotaxis in vitro and in vivo.²¹⁻²³ CD11b+ cells expressed 20-fold more p110γ than p110α, β or δ (FIGS. 29a, 42a-b). siRNA mediated knockdown of p110γ, but not of p110α, β or δ, suppressed myeloid cell adhesion (FIG. 29b; FIG. 43a-b). Additionally, selective inhibitors of PI3-kinase γ,²⁴⁻²⁷ but not of other isoforms, suppressed chemoattractant-stimulated PI3-kinase catalytic activity and inhibited myeloid cell adhesion with IC₅₀s from 50-158 nM (FIGS. 29b, 41a-e). p110γ−/− myeloid cells²¹⁻²³ also failed to adhere to EC (FIG. 29a) and exhibited loss of PI3-kinase catalytic activity (FIG. 41e), but expressed normal levels of integrin α4 (FIG. 42c). As ligand binding and integrin activation were also suppressed by PI3-kinase γ inhibitors and in p110γ−/− myeloid cells (FIG. 42d-i), these studies indicate that PI3-kinase γ regulates integrin α4β1 activation.

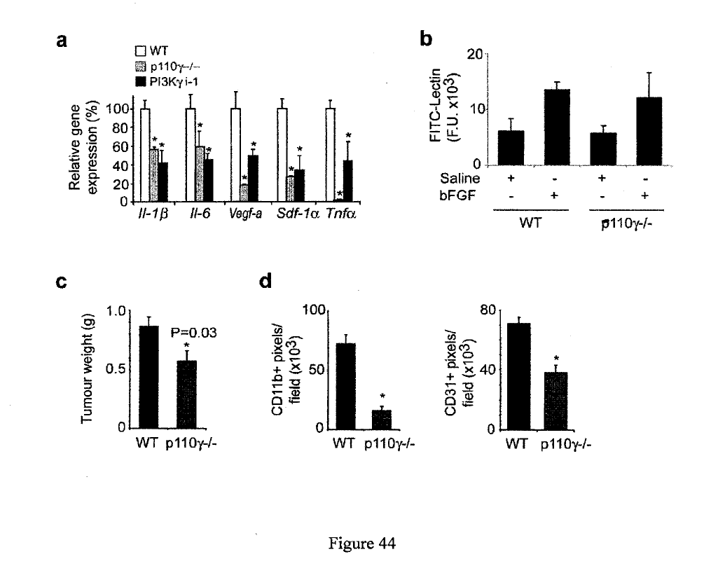

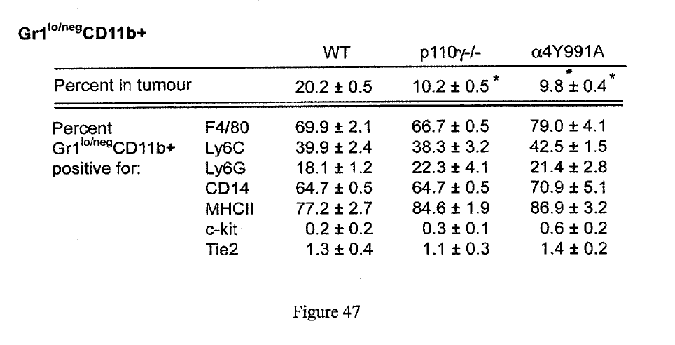

Adoptively transferred PI3-kinase γ inhibitor and siRNA treated myeloid cells, as well as p110 γ−/− myeloid cells, failed to arrest in tumours (FIGS. 29c, 42f, 43c). In contrast, myeloid cells treated with PI3-kinase α and β inhibitors or α, β and δ siRNAs arrested in tumours (FIGS. 29c, 42f, 43c). Importantly, daily dosing of mice bearing LLC tumours with a PI3-kinase γ inhibitor, TG100-115²⁵⁻²⁷ (PI3Kγi-1), suppressed lung carcinoma growth with an IC₅₀ of 0.5 mg/ml (FIGS. 29d, 41c). A second inhibitor, AS605240²⁴ (PI3Kγi-2), suppressed tumour growth at similar doses (FIG. 29d). Although TG100-115 has a short half-life in vivo (t_{1/2}=0.22 h), it rapidly and sustainably inhibits myeloid cell PI3kinase catalytic activity in vivo (FIG. 41d-f). Ablation of PI3-kinase γ expression as well as PI3-kinase γ inhibitors suppressed LLC and B16 melanoma growth, inflammation and angiogenesis, and blocked expression of pro-angiogenic and inflammatory factors (FIG. 29e; FIG. 44a,c-e). Whereas 20% of the tumour is composed of myeloid cells in WT animals, only 10% of the tumour is composed of myeloid cells in p110γ−/− and integrin α4Y991A animals (FIG. 47). As growth factor-induced angiogenesis occurs normally in p110γ−/− mice (FIG. 44b) and in the presence of PI3-kinase γ inhibitors²⁷, these studies demonstrate PI3-kinase γ modulates tumour growth by activating integrin α4 during tumour inflammation in vivo.

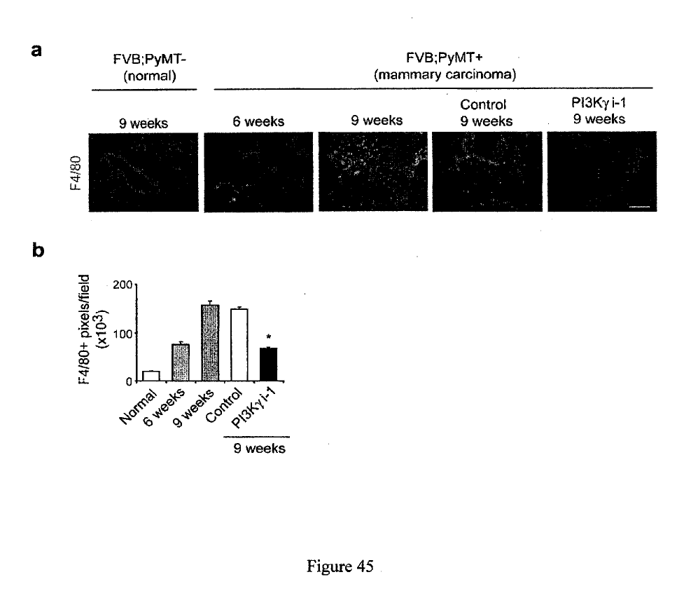

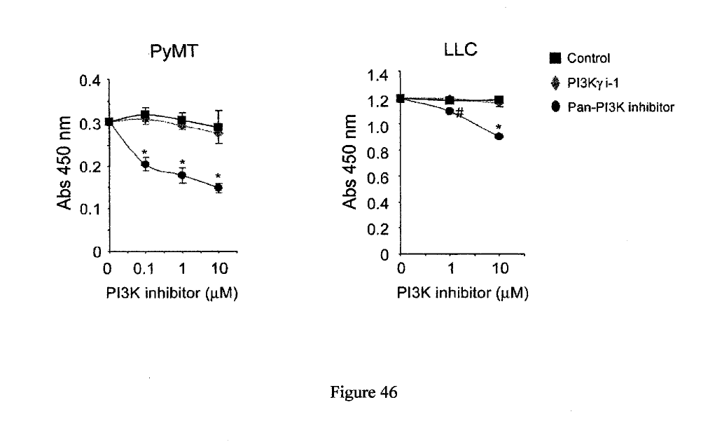

PI3-kinase γ inhibitors also suppressed spontaneous murine breast tumour progression. When treated with these inhibitors, PyMT breast tumour growth and progression was strongly inhibited (FIG. 29f-g) as control treated animals exhibited 28+/−6.5% carcinoma, 33+/−7.3% hyperplasia, and 38+/−5% normal tissue while PI3Kγi treated animals exhibited 6+/−1.3% carcinoma, 27+/−5% hyperplasia and 67+/−3% normal tissue. PI3-kinase γ inhibitors blocked CD11b+ cell and macrophage invasion as well as angiogenesis in breast tissues (FIGS. 29h, 45a-b) without directly affecting tumour cell proliferation (FIG. 46a-b). These studies demonstrate that PI3-kinase γ inhibitors could be useful in controlling spontaneous tumour growth and malignancy.

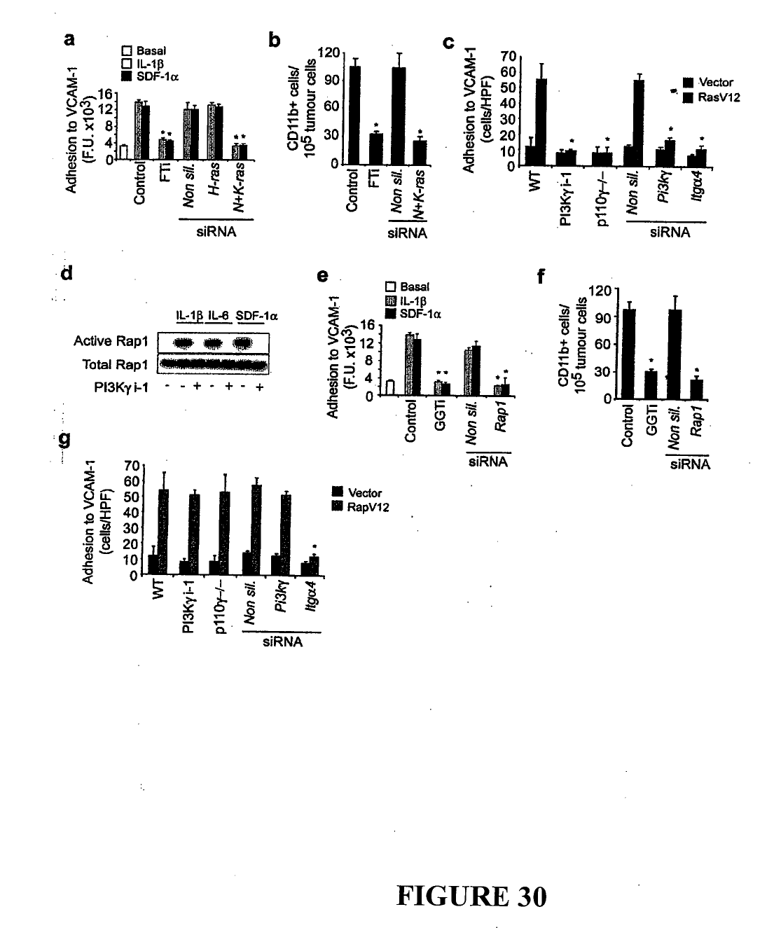

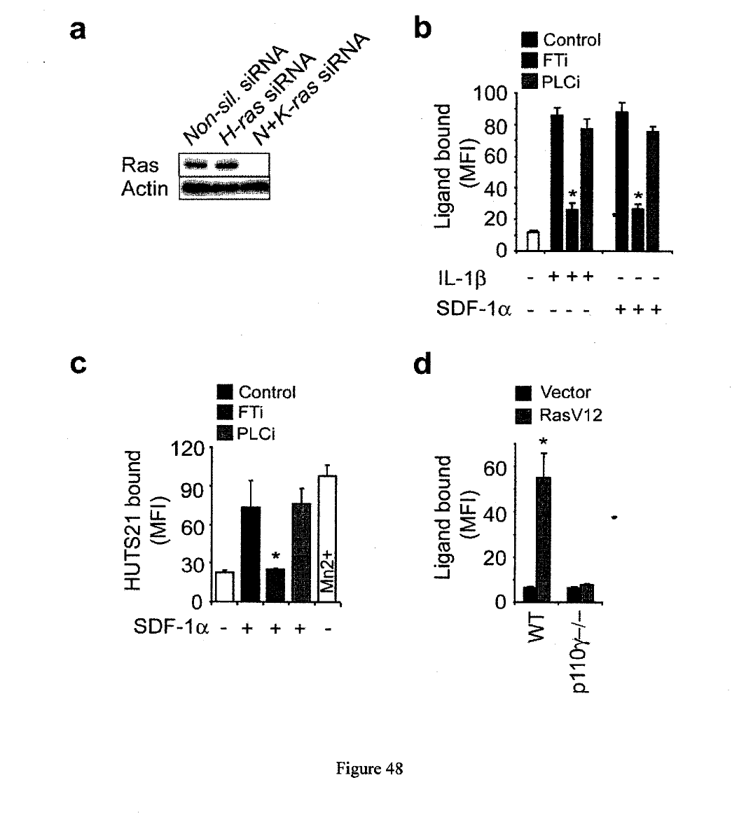

As many chemoattractant receptors activate Ras and prior studies established roles for Ras and p101 in PI3-kinase γ activation²⁸, we investigated the role of Ras in myeloid cell adhesion. siRNA-mediated knockdown of N+K-Ras but not H-Ras suppressed Ras expression and chemokine-stimulated myeloid cell adhesion (FIGS. 30a, 48a). Farnesyl transferase inhibitors (FTi), which block Ras prenylation, also blocked adhesion, ligand binding and integrin conformational changes (FIGS. 30a, 48b-c). N+K-Ras siRNAs and FTi also suppressed myeloid cell arrest in tumours in vivo (FIG. 30b). Importantly, expression of activated Ras (RasV12) stimulated chemokine-independent adhesion (FIG. 30c) and ligand binding (FIG. 48d). However, RasV12 was unable to stimulate adhesion in α4 or PI3-kinase γ siRNA transfected, PI3-kinase γ inhibitor-treated or p110γ−/− cells (FIGS. 30c, 48d). Thus, N- and K-Ras are required for PI3-kinase γ-dependent integrin α4β1 activation and myeloid cell adhesion in vitro and in vivo.

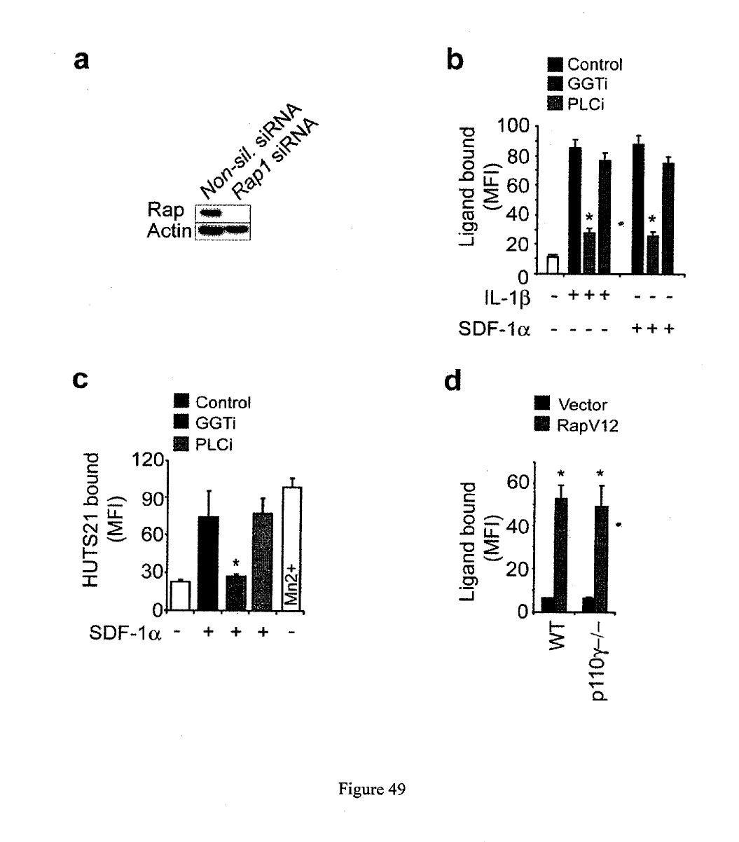

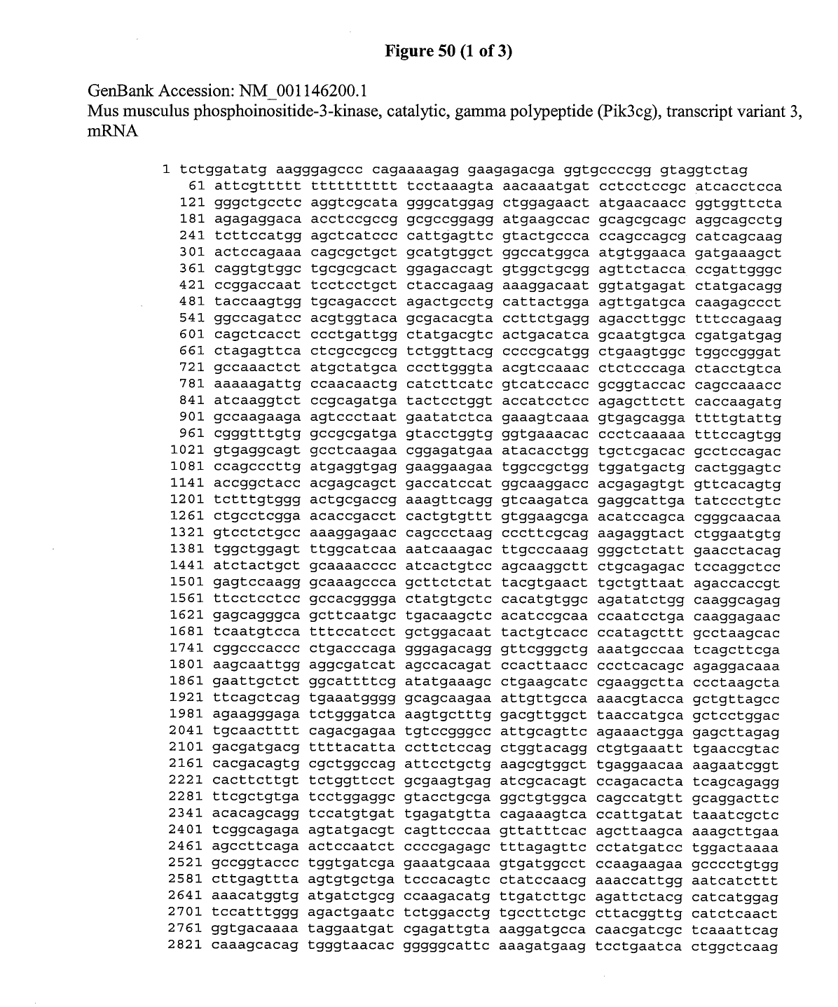

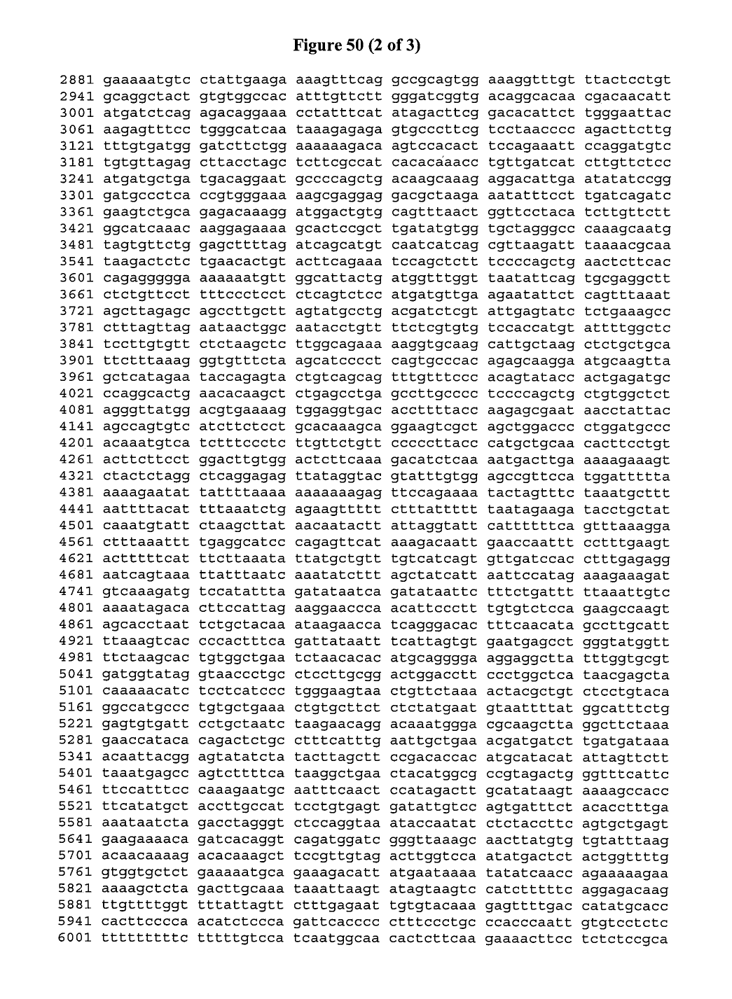

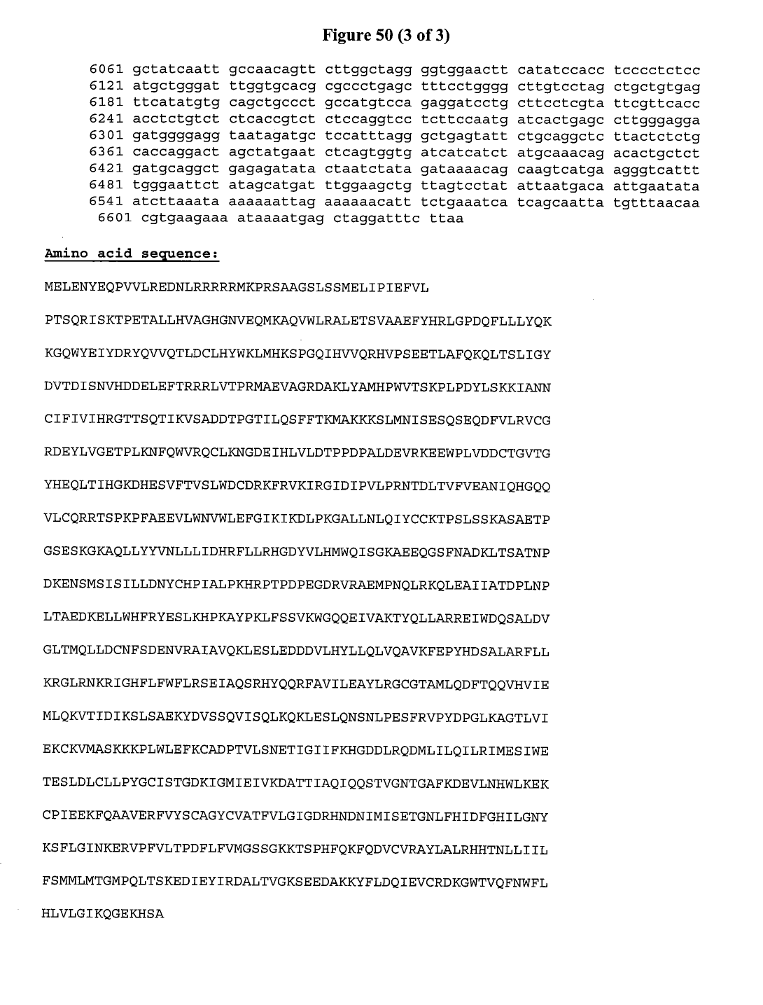

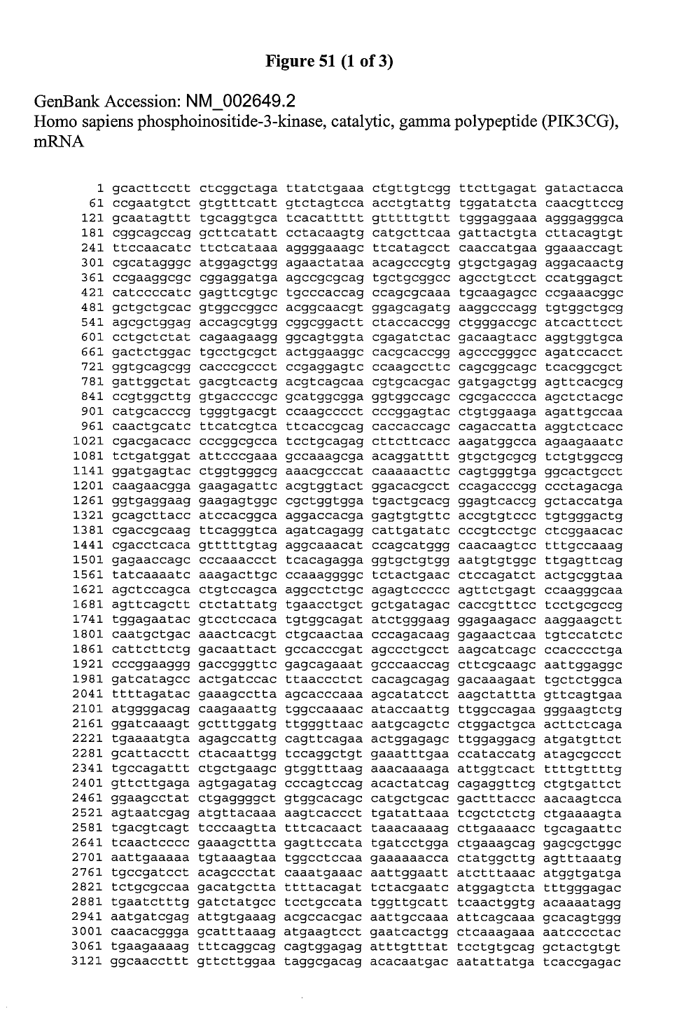

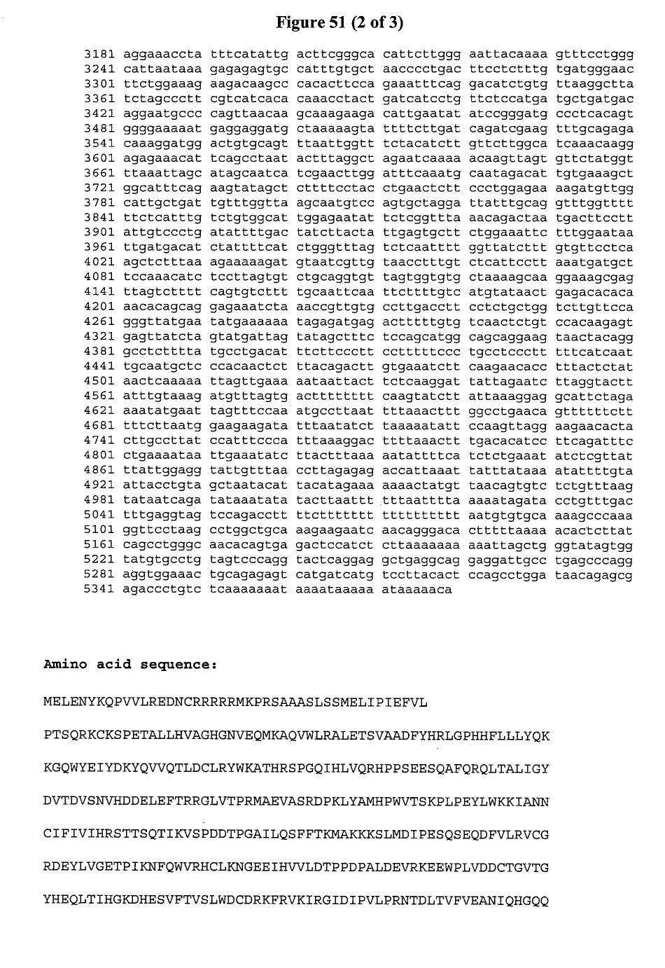

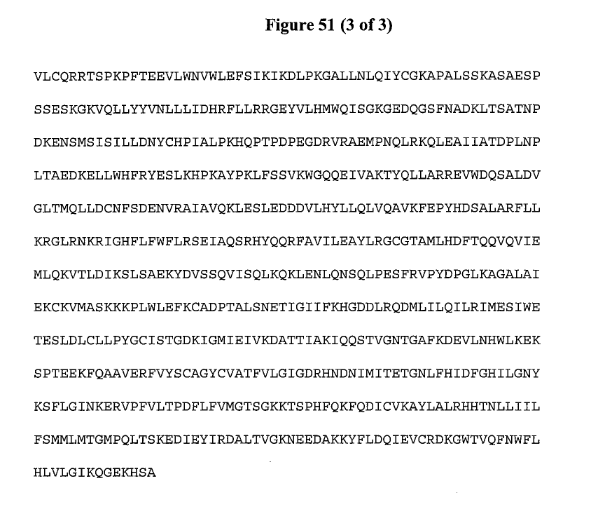

The small GTPase Rap1 can regulate integrin activation by promoting talin binding to integrin beta chain cytoplasmic tails, thereby disrupting a salt bridge between alpha and beta chains and inducing integrin conformational changes²⁹. Diverse chemoattractants stimulated myeloid cell Rap1a activation, which was suppressed by PI3-kinase γ inhibition (FIGS. 30d, 49a). Rap1a siRNA and geranylgeranyltransferase inhibitors (GGTi) suppressed adhesion, ligand binding and integrin conformational changes, as well as arrest of myeloid cells in tumours (FIG. 30e-f, 49b-c). In contrast, expression of activated Rap1 (RapV12) stimulated chemoattractant-independent adhesion to VCAM-1 (FIG. 30g) and ligand binding (FIG. 49d), indicating that Rap1 is sufficient to activate integrin α4. RapV12 stimulated adhesion in p110γ−/− and PI3-kinase γ siRNA, but not integrin α4 siRNA, transfected cells, indicating that Rap1 activates integrin α4β31 downstream of PI3-kinase γ. Although it is currently unclear how PI3-kinase γ activates Rap1, it may modulate Rap1 regulatory factors, such as (CalDAG)-GEFI (calcium and diacylglycerol (CalDAG)-GEFI), which is required for leukocyte integrin activation³⁰.

Prior studies have shown that tumour-derived chemoattractants can promote tumour inflammation or progression¹⁻¹¹. Our studies reveal that a common Ras-PI3-kinase γ-integrin α4β1 signalling pathway regulates tumour inflammation and progression, regardless of the chemoattractants expressed (FIG. 31). These studies indicate that therapeutic agents directed at inhibiting PI3-kinase γ or integrin α4 could be useful in suppressing tumour inflammation, growth and progression.

### REFERENCES REFERRED TO IN THE TEXT AND EXAMPLES 1-8

1. Lin, W. W. & Karin, M. A cytokine-mediated link between innate immunity, inflammation, and cancer. J. Clin. Invest. 117, 1175-1183 (2007).
2. Yang, L. et al. Expansion of myeloid immune suppressor Gr+CD11b+ cells in tumor-bearing host directly promotes tumor angiogenesis. Cancer Cell 6, 409-421 (2004).
3. Lin, E. Y., et al. Macrophages regulate the angiogenic switch in a mouse model of breast cancer. Cancer Res. 66, 11238-11246 (2006).
4. De Palma, M. et al. Tie2 identifies a hematopoietic lineage of proangiogenic monocytes required for tumor vessel formation and a mesenchymal population of pericyte progenitors. Cancer Cell 8, 211-226 (2005).
5. Grunewald, M. et al. VEGF-induced adult neovascularization: recruitment, retention, and role of accessory cells. Cell 124, 175-189 (2006).
6. Du, R. et al. HIF1alpha induces the recruitment of bone marrow-derived vascular modulatory cells to regulate tumor angiogenesis and invasion. Cancer Cell 13, 206-220 (2008).
7. Ahn, G. O., & Brown, J. M. Matrix metalloproteinase-9 is required for tumor vasculogenesis but not for angiogenesis: role of bone marrow-derived myelomonocytic cells. Cancer Cell 13, 193-205 (2008).
8. Shojaei, F. et al. Bv8 regulates myeloid-cell-dependent tumour angiogenesis. Nature 450, 825-831 (2007).
9. Zacchigna, S. et al. Bone marrow cells recruited through the neuropilin-1 receptor promote arterial formation at the sites of adult neoangiogenesis in mice. J. Clin. Invest. 118, 2062-2075 (2008).
10. Bunt, S. K. et al. Inflammation induces myeloid-derived suppressor cells that facilitate tumor progression. J. Immunol. 176, 284-290 (2006).
11. Bunt, S. K. et al. Reduced inflammation in the tumor microenvironment delays the accumulation of myeloid-derived suppressor cells and limits tumor progression. Cancer Res. 67, 10019-10026 (2007).
12. Nishie, et al. Macrophage infiltration and heme oxygenase-1 expression correlate with angiogenesis in human gliomas. Clin. Cancer Res. 5, 1107-1113 (1999).
13. Niwa, Y., et al. Correlation of tissue and plasma RANTES levels with disease course in patients with breast or cervical cancer. Clin. Cancer Res. 2, 285-289 (2001).
14. Tsutsui, S., et al. Macrophage infiltration and its prognostic implications in breast cancer: the relationship with VEGF expression and microvessel density. Oncol. Rep. 14, 425-431 (2005).
15. Ueno, T., et al. Significance of macrophage chemoattractant protein-1 in macrophage recruitment, angiogenesis, and survival in human breast cancer. Clin. Cancer Res. 8, 3282-3289 (2000).
16. Lobb, R. R. & Hemler, M. E. The pathophysiologic role of alpha 4 integrins in vivo. J. Clin. Invest. 94, 1722-1728 (1994).
17. Rose, D. M., Alon, R., & Ginsberg, M. H. Integrin modulation and signaling in leukocyte adhesion and migration. Immunol. Rev. 218, 126-134 (2007).
18. Grabovsky, V. et al. Subsecond induction of alpha4 integrin clustering by immobilized chemokines stimulates leukocyte tethering and rolling on endothelial vascular cell adhesion molecule 1 under flow conditions. J. Exp. Med. 192, 495-506 (2000).
19. Jin, H., Su J., Garmy-Susini, B. Kleeman, J., & Varner, J. Integrin alpha4beta1 promotes monocyte trafficking and angiogenesis in tumors. Cancer Res. 66, 2146-2152 (2006).
20. Burns, K. et al. MyD88, an adapter protein involved in interleukin-1 signaling. J. Biol. Chem. 273, 12203-12209 (1998).
21. Adachi, O., et al. Targeted disruption of the MyD88 gene results in loss of IL-1- and IL-18-mediated function. Immunity 9, 143-150 (1998).
22. Donzella, G. A. et al. AMD3100, a small molecule inhibitor of HIV-1 entry via the CXCR4 co-receptor. Nat. Med. 4, 72-77 (1998)
23. Doukas J. et al. Phosphoinositide 3-kinase gamma/delta inhibition limits infarct size after myocardial ischemia/reperfusion injury. Proc. Natl. Acad. Sci. USA. 103, 19866-19871 (2006).
24. Serban, D., Leng, J., & Cheresh, D. H-ras regulates angiogenesis and vascular permeability by activation of distinct downstream effectors. Circ. Res. 102, 1350-1358. (2008).
25. Arnaout, M. A., Mahalingam, B., & Xiong, J. P. Integrin structure, allostery, and bidirectional signaling. Annu. Rev. Cell Dev. Biol. 21, 381-410 (2005).
26. Luque, A. et al. Activated conformations of very late activation integrins detected by a group of antibodies (HUTS) specific for a novel regulatory region (355-425) of the common beta 1 chain. J. Biol. Chem. 271, 11067-11075 (1996).
27. Liu, S. et al. Binding of paxillin to alpha4 integrins modifies integrin-dependent biological responses. Nature 402, 676-681 (1999).
28. Alon, R. et al. Alpha4beta1-dependent adhesion strengthening under mechanical strain is regulated by paxillin association with the alpha4-cytoplasmic domain. J. Cell Biol. 171, 1073-1084 (2005).
29. Manevich, E., Grabovsky, V., Feigelson, S. W. & Alon, R. Talin 1 and paxillin facilitate distinct steps in rapid VLA-4-mediated adhesion strengthening to vascular cell adhesion molecule 1. J. Biol. Chem. 282, 25338-25348 (2007).
30. Feral, C. C., et al. Blocking the alpha 4 integrin-paxillin interaction selectively impairs mononuclear leukocyte recruitment to an inflammatory site. J. Clin. Invest. 116, 715-723 (2006).

### REFERENCES REFERRED TO IN EXAMPLE 10

1. Lin, W. W. & Karin, M. A cytokine-mediated link between innate immunity, inflammation, and cancer. J. Clin. Invest. 117, 1175-1183 (2007).
2. Grabrilovich, D. I. & Nagaraj, S. Myeloid-derived suppressor cells as regulators of the immune system. Nature Reviews Immunology 9, 162-174 (2009).
3. Murdock, C., Muthana, M., Coffelt, S. B., & Lewis, C. E. The role of myeloid cells in the promotion of tumour angiogenesis. Nature Reviews Cancer 8, 618-631 (2008).
4. Yang, L. et al. Expansion of myeloid immune suppressor Gr+CD11b+ cells in tumor-bearing host directly promotes tumor angiogenesis. Cancer Cell 6, 409-421 (2004).
5. Lin, E. Y., et al. Macrophages regulate the angiogenic switch in a mouse model of breast cancer. Cancer Res. 66, 11238-11246 (2006).
6. De Palma, M. et al. Tie2 identifies a hematopoietic lineage of proangiogenic monocytes required for tumor vessel formation and a mesenchymal population of pericyte progenitors. Cancer Cell 8, 211-226 (2005).
7. Grunewald, M. et al. VEGF-induced adult neovascularization: recruitment, retention, and role of accessory cells. Cell 124, 175-189 (2006).
8. Du, R. et al. HIF1alpha induces the recruitment of bone marrow-derived vascular modulatory cells to regulate tumor angiogenesis and invasion. Cancer Cell 13, 206-220 (2008).
9. Shojaei, F. et al. Bv8 regulates myeloid-cell-dependent tumour angiogenesis. Nature 450, 825-831 (2007).
10. Bunt, S. K. et al. Inflammation induces myeloid-derived suppressor cells that facilitate tumor progression. J. Immunol. 176, 284-290 (2006).
11. Kim, S., et al. Carcinoma-produced factors activate myeloid cells through TLR2 to stimulate metastasis. Nature 457, 102-106 (2009).
12. Lobb, R. R. & Hemler, M. E. The pathophysiologic role of alpha 4 integrins in vivo. J. Clin. Invest. 94, 1722-1728 (1994).
13. Rose, D. M., Alon, R., & Ginsberg, M. H. Integrin modulation and signaling in leukocyte adhesion and migration. Immunol. Rev. 218, 126-134 (2007).
14. Konradi, A. W., Pleiss M. A., Semko, C. M., Yednock T, Smith J. L. Multimeric VLA-4 antagonists comprising polymer moieties. US 2006/0013799 A1 (2006).
15. Jin, H., Su J., Garmy-Susini, B. Kleeman, J., & Varner, J. Integrin alpha4beta1 promotes monocyte trafficking and angiogenesis in tumors. Cancer Res. 66, 2146-2152 (2006).
16. Feral, C. C., et al. Blocking the alpha 4 integrin-paxillin interaction selectively impairs mononuclear leukocyte recruitment to an inflammatory site. J. Clin. Invest. 116, 715-723 (2006).
17. Manevich, E., Grabovsky, V., Feigelson, S. W. & Alon, R. Talin 1 and paxillin facilitate distinct steps in rapid VLA-4-mediated adhesion strengthening to vascular cell adhesion molecule 1. J. Biol. Chem. 282, 25338-25348 (2007).
18. Scott, L. M., Priestley, G. V. & Papayannopoulou, T. Deletion of alpha4 integrins from adult hematopoietic cells reveals roles in homeostasis, regeneration, and homing. Mol Cell Biol. 23, 9349-9360 (2003).
19. Arnaout, M. A., Mahalingam, B., & Xiong, J. P. Integrin structure, allostery, and bidirectional signaling. Annu. Rev. Cell Dev. Biol. 21, 381-410 (2005).
20. Luque, A. et al. Activated conformations of very late activation integrins detected by a group of antibodies (HUTS) specific for a novel regulatory region (355-425) of the common beta 1 chain. J. Biol. Chem. 271, 11067-11075 (1996).
21. Sasaki, T, et al. Function of PMK7 in thymocyte development, T cell activation, and neutrophil migration. Science 287, 1040-1046 (2000).
22. Li, Z. et al. Roles of PLC-β2 and -β3 and PI3kγ in chemoattractant mediated signal transduction. Science 287, 1046-1049 (2000).
23. Hirsch, E. et al. Central role for G protein coupled phosphoinositide 3-kinase γ in inflammation. Science 287, 1049-1053 (2000).
24. Camps, M. et al. Blockade of PI3K γ suppresses joint inflammation and damage in mouse models of rheumatoid arthritis. Nature Medicine 11, 936-943 (2005).
25. Palanki, M. S., et al. Discovery of 3,3′-(2,4-diaminopteridine-6,7-diyl)diphenol as an isozyme-selective inhibitor of PI3K for the treatment of ischemia reperfusion injury associated with myocardial infarction. Journal of Medicinal Chemistry 50, 4279-4294 (2007).
26. Doukas, J. et al. Phosphoinositide 3-kinase gamma/delta inhibition limits infarct size after myocardial ischemia/reperfusion injury. Proc. Natl. Acad. Sci. USA. 103, 19866-19871 (2006).
27. Serban, D., Leng, J., & Cheresh, D. H-ras regulates angiogenesis and vascular permeability by activation of distinct downstream effectors. Circ. Res. 102, 1350-1358. (2008).
28. Suire, S. et al. Gbetagammas and the Ras binding domain of p110gamma are both important regulators of PI(3)Kgamma signalling in neutrophils. Nat. Cell Biol. 8, 1303-1309 (2006).
29. Lee, H. S., Lim, C. J., Puzon-McLaughlin, W., Shattil, S. J., & Ginsberg, M. H. RIAM activates integrins by linking talin to ras GTPase membrane-targeting sequences. J. Biol. Chem. 284, 5119-5127 (2009).
30. Bergmeier, W., et al. Mice lacking the signaling molecule CalDAG-GEFI represent a model for leukocyte adhesion deficiency type III. J. Clin. Invest. 117, 1699-1707 (2007).

### REFERENCES REFERRED TO IN EXAMPLE 9

31) Davie, S. A., et al. Effects of FVB/NJ and C57Bl/6J strain backgrounds on mammary tumor phenotype in inducible nitric oxide synthase deficient mice. Transgenic Res. 16, 193-201 (2007).
32) Schmittgen, T. D. & Livak, K. J. Analyzing real-time PCR data by the comparative C(T) method. Nat. Protoc. 3, 1101-8 (2008).

Each and every publication and patent mentioned in the above specification is herein incorporated by reference in its entirety for all purposes. Various modifications and variations of the described methods and system of the invention will be apparent to those skilled in the art without departing from the scope and spirit of the invention.

Although the invention has been described in connection with specific embodiments, the invention as claimed should not be unduly limited to such specific embodiments. Indeed, various modifications of the described modes for carrying out the invention which are obvious to those skilled in the art and in fields related thereto are intended to be within the scope of the following claims.

We claim:

1. A method for inhibiting cancer in a subject comprising administering to a subject having cancer that comprises endothelial cells a therapeutically effective amount of a PI-3-kinase gamma inhibitor that reduces at least one of
(a) adhesion of myeloid cells to said endothelial cells,
(b) migration of myeloid cells into said cancer,
(c) growth of said cancer,
(d) activation of integrin a4b1 that is comprised on said myeloid cells, and
(e) clustering of integrin a4b1 that is comprised on said myeloid cells,
wherein said PI-3-kinase gamma inhibitor comprises an antibody that specifically binds to PI-3-kinase gamma.

2. A method for inhibiting cancer in a subject comprising administering to a subject having cancer that comprises endothelial cells a therapeutically effective amount of a PI-3-kinase gamma inhibitor that reduces at least one of
(f) adhesion of myeloid cells to said endothelial cells,
(g) migration of myeloid cells into said cancer,
(h) growth of said cancer,
(i) activation of integrin a4b1 that is comprised on said myeloid cells, and
(j) clustering of integrin a4b1 that is comprised on said myeloid cells,
wherein said PI-3-kinase gamma inhibitor comprises a nucleic acid sequence selected from PI-3-kinase gamma antisense sequence and PI-3-kinase gamma ribozyme sequence.
